SEQUENCE LISTING (54 sequences)

SEQ ID NO: 1 - protein, 879 aa, Homo sapiens
  source 1..879: note = The PTGFRN Protein
MGRLASRPLLLALLSLALCRGRVVRVPTATLVRVVGTELVIPCNVSDYDGPSEQNFDWSFSSLGSSFVELASTWEVGFPAQLYQERLQRGEILLRRTANDAVELHIKNVQPSDQGHYKCSTPSTDATVQGNYEDTVQVKVLADSLHVGPSARPPPSLSLREGEPFELRCTAASASPLHTHLALLWEVHRGPARRSVLALTHEGRFHPGLGYEQRYHSGDVRLDTVGSDAYRLSVSRALSADQGSYRCIVSEWIAEQGNWQEIQEKAVEVATVVIQPSVLRAAVPKNVSVAEGKELDLTCNITTDRADDVRPEVTWSFSRMPDSTLPGSRVLARLDRDSLVHSSPHVALSHVDARSYHLLVRDVSKENSGYYYCHVSLWAPGHNRSWHKVAEAVSSPAGVGVTWLEPDYQVYLNASKVPGFADDPTELACRVVDTKSGEANVRFTVSWYYRMNRRSDNVVTSELLAVMDGDWTLKYGERSKQRAQDGDFIFSKEHTDTFNFRIQRTTEEDRGNYYCVVSAWTKQRNNSWVKSKDVFSKPVNIFWALEDSVLVVKARQPKPFFAAGNTFEMTCKVSSKNIKSPRYSVLIMAEKPVGDLSSPNETKYIISLDQDSVVKLENWTDASRVDGVVLEKVQEDEFRYRMYQTQVSDAGLYRCMVTAWSPVRGSLWREAATSLSNPIEIDFQTSGPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEMD

SEQ ID NO: 2-32 - skipped (31 entries)

SEQ ID NO: 33 - protein, 192 aa, synthetic construct
  source 1..192: note = The PTGFRN Protein Fragment
GPIFNASVHSDTPSVIRGDLIKLFCIITVEGAALDPDDMAFDVSWFAVHSFGLDKAPVLLSSLDRKGIVTTSRRDWKSDLSLERVSVLEFLLQVHGSEDQDFGNYYCSVTPWVKSPTGSWQKEAEIHSKPVFITVKMDVLNAFKYPLLIGVGLSTVIGLLSCLIGYCSSHWCCKKEVQETRRERRRLMSMEM

SEQ ID NO: 34-48 - skipped (15 entries)

SEQ ID NO: 49 - protein, 227 aa, synthetic construct
  source 1..227: note = The BASP1 Protein
MGGKLSKKKKGYNVNDEKAKEKDKKAEGAATEEEGTPKESEPQAAAEPAEAKEGKEKPDQDAEGKAEEKEGEKDAAAAKEEAPKAEPEKTEGAAEAKAEPPKAPEQEQAAPGPAAGGEAPKAAEAAAAPAESAAPAAGEEPSKEEGEPKKTEAPAAPAAQETKSDGAPASDSKPGSSEAAPSSKETPAATEAPSSTPKAQGPAASAEEPKPVEAPAANSDQTVTVKE

SEQ ID NO: 50 - protein, 888 aa, synthetic construct [sequence omitted]

SEQ ID NO: 51 - protein, 835 aa, synthetic construct
  source 1..835: note = HMGCR Isoform 2 (identifier: P04035-2) (also known as HMGCR-1c)
MLSRLFRMHGLFVASHPWEVIVGTVTLTICMMSMNMFTGNNKICGWNYECPKFEEDVLSSDIIILTITRCIAILYIYFQFQNLRQLGSKYILGIAGLFTIFSSFVFSTVVIHFLDKELTGLNEALPFFLLLIDLSRASTLAKFALSSNSQDEVRENIARGMAILGPTFTLDALVECLVIGVGTMSGVRQLEIMCCFGCMSVLANYFVFMTFFPACVSLVLELSRESREGRPIWQLSHFARVLEEEENKPNPVTQRVKMIMSLGLVLVHAHSRWIADPSPQNSTADTSKVSLGLDENVSKRIEPSVSLWQFYLSKMISMDIEQVITLSLALLLAVKYIFFEQTETESTLSLKNPITSPVVTQKKVPDNCCRREPMLVRNNQKCDSVEEETGINRERKVEVIKPLVAETDTPNRATFVVGNSSLLDTSSVLVTQEPEIELPREPRPNEECLQILGNAEKGAKFLSDAEIIQLVNAKHIPAYKLETLMETHERGVSIRRQLLSKKLSEPSSLQYLPYRDYNYSLLGGGASSRVLADGMTRGPVVRLPRACDSAEVKAWLETSEGFAVIKEAFDSTSRFARLQKLHTSIAGRNLYIRFQSRSGDAMGMNMISKGTEKALSKLHEYFPEMQILAVSGNYCTDKKPAAINWIEGRGKSVVCEAVIPAKVVREVLKTTTEAMIEVNINKNLVGSAMAGSIGGYNAHAANIVTAIYIACGQDAAQNVGSSNCITLMEASGPTNEDLYISCTMPSIEIGTVGGGTNLLPQQACLQMLGVQGACKDNPGENARQLARIVCGTVMAGELSLMAALAAGHLVKSHMIHNRSKINLQDLQGACTKKTA

SEQ ID NO: 52 - protein, 908 aa, synthetic construct
  source 1..908: note = HMGCR Isoform 3 (identifier: Q92570-3) (also known as HMGCR-1b)
MQWMSHTRERDAGSKDSVATMLSRLFRMHGLFVASHPWEVIVGTVTLTICMMSMNMFTGNNKICGWNYECPKFEEDVLSSDIIILTITRCIAILYIYFQFQNLRQLGSKYILGIAGLFTIFSSFVFSTVVIHFLDKELTGLNEALPFFLLLIDLSRASTLAKFALSSNSQDEVRENIARGMAILGPTFTLDALVECLVIGVGTMSGVRQLEIMCCFGCMSVLANYFVFMTFFPACVSLVLELSRESREGRPIWQLSHFARVLEEEENKPNPVTQRVKMIMSLGLVLVHAHSRWIADPSPQNSTADTSKVSLGLDENVSKRIEPSVSLWQFYLSKMISMDIEQVITLSLALLLAVKYIFFEQTETESTLSLKNPITSPVVTQKKVPDNCCRREPMLVRNNQKCDSVEEETGINRERKVEVIKPLVAETDTPNRATFVVGNSSLLDTSSVLVTQEPEIELPREPRPNEECLQILGNAEKGAKFLSDAEIIQLVNAKHIPAYKLETLMETHERGVSIRRQLLSKKLSEPSSLQYLPYRDYNYSLVMGACCENVIGYMPIPVGVAGPLCLDEKEFQVPMATTEGCLVASTNRGCRAIGLGGGASSRVLADGMTRGPVVRLPRACDSAEVKAWLETSEGFAVIKEAFDSTSRFARLQKLHTSIAGRNLYIRFQSRSGDAMGMNMISKGTEKALSKLHEYFPEMQILAVSGNYCTDKKPAAINWIEGRGKSVVCEAVIPAKVVREVLKTTTEAMIEVNINKNLVGSAMAGSIGGYNAHAANIVTAIYIACGQDAAQNVGSSNCITLMEASGPTNEDLYISCTMPSIEIGTVGGGTNLLPQQACLQMLGVQGACKDNPGENARQLARIVCGTVMAGELSLMAALAAGHLVKSHMIHNRSKINLQDLQGACTKKTA

SEQ ID NO: 53 - protein, 1141 aa, synthetic construct
  source 1..1141: note = SREBF2 Isoform 1 (identifier: Q12772-1)
MDDSGELGGLETMETLTELGDELTLGDIDEMLQFVSNQVGEFPDLFSEQLCSSFPGSGGSGSSSGSSGSSSSSSNGRGSSSGAVDPSVQRSFTQVTLPSFSPSAASPQAPTLQVKVSPTSVPTTPRATPILQPRPQPQPQPQTQLQQQTVMITPTFSTTPQTRIIQQPLIYQNAATSFQVLQPQVQSLVTSSQVQPVTIQQQVQTVQAQRVLTQTANGTLQTLAPATVQTVAAPQVQQVPVLVQPQIIKTDSLVLTTLKTDGSPVMAAVQNPALTALTTPIQTAALQVPTLVGSSGTILTTMPVMMGQEKVPIKQVPGGVKQLEPPKEGERRTTHNIIEKRYRSSINDKIIELKDLVMGTDAKMHKSGVLRKAIDYIKYLQQVNHKLRQENMVLKLANQKNKLLKGIDLGSLVDNEVDLKIEDFNQNVLLMSPPASDSGSQAGFSPYSIDSEPGSPLLDDAKVKDEPDSPPVALGMVDRSRILLCVLTFLCLSFNPLTSLLQWGGAHDSDQHPHSGSGRSVLSFESGSGGWFDWMMPTLLLWLVNGVIVLSVFVKLLVHGEPVIRPHSRSSVTFWRHRKQADLDLARGDFAAAAGNLQTCLAVLGRALPTSRLDLACSLSWNVIRYSLQKLRLVRWLLKKVFQCRRATPATEAGFEDEAKTSARDAALAYHRLHQLHITGKLPAGSACSDVHMALCAVNLAECAEEKIPPSTLVEIHLTAAMGLKTRCGGKLGFLASYFLSRAQSLCGPEHSAVPDSLRWLCHPLGQKFFMERSWSVKSAAKESLYCAQRNPADPIAQVHQAFCKNLLERAIESLVKPQAKKKAGDQEEESCEFSSALEYLKLLHSFVDSVGVMSPPLSRSSVLKSALGPDIICRWWTSAITVAISWLQGDDAAVRSHFTKVERIPKALEVTESPLVKAIFHACRAMHASLPGKADGQQSSFCHCERASGHLWSSLNVSGATSDPALNHVVQLLTCDLLLSLRTALWQKQASASQAVGETYHASGAELAGFQRDLGSLRRLAHSFRPAYRKVFLHEATVRLMAGASPTRTHQLLEHSLRRRTTQSTKHGEVDAWPGQRERATAILLACRHLPLSFLSSPGQRAVLLAEAARTLEKVGDRRSCNDCQQMIVKLGGGTAIAAS

SEQ ID NO: 54 - protein, 681 aa, synthetic construct
  source 1..681: note = SREBF2 Isoform 2 (identifier: Q12772-2)
MDDSGELGGLETMETLTELGDELTLGDIDEMLQFVSNQVGEFPDLFSEQLCSSFPGSGGSGSSSGSSGSSSSSSNGRGSSSGAVDPSVQRSFTQVTLPSFSPSAASPQAPTLQVKVSPTSVPTTPRATPILQPRPQPQPQPQTQLQQQTVMITPTFSTTPQTRIIQQPLIYQNAATSFQVLQPQVQSLVTSSQVQPVTIQQQVQTVQAQRVLTQTANGTLQTLAPATVQTVAAPQVQQVPVLVQPQIIKTDSLVLTTLKTDGSPVMAAVQNPALTTPIQTAALQVPTLVGSSGTILTTMPVMMGQEKVPIKQVPGGVKQLEPPKEGERRTTHNIIEKRYRSSINDKIIELKDLVMGTDAKMHKSGVLRKAIDYIKYLQQVNHKLRQENMVLKLANQKNKLLKGIDLGSLVDNEVDLKIEDFNQNVLLMSPPASDSGSQAGFSPYSIDSEPGSPLLDDAKVKDEPDSPPVALGMVDRSRILLCVLTFLCLSFNPLTSLLQWGGAHDSDQHPHSGSGRSVLSFESGSGGWFDWMMPTLLLWLVNGVIVLSVFVKLLVHGEPVIRPHSRSSVTFWRHRKQADLDLARGVYGKKSGATHSIEEELNIHISRGTRTRTLLSSRRFCSCCRQPTNLPGSFGPGTAHLPPGPGLQPLLERDPLQPAEATPGALAAQESLPVPAGHASH

(19) United States
(12) Patent Application Publication
(10) Pub. No.: US 2024/0336896 A1
(43) Pub. Date: Oct. 10, 2024

(54) MODIFIED PRODUCER CELLS FOR EXTRACELLULAR VESICLE PRODUCTION

(71) Applicant: LONZA SALES AG, Basel (CH)

(72) Inventors: Shelly A. MARTIN, Cambridge, MA (US); Russell E. MCCONNELL, Cambridge, MA (US); Jonathan FINN, Cambridge, MA (US); Scott D. ESTES, Cambridge, MA (US)

(21) Appl. No.: 18/700,035

(22) PCT Filed: Oct. 14, 2022
(86) PCT No.: PCT/US2022/078150

§ 371 Date: Apr. 10, 2024

Related U.S. Application Data
(60) Provisional application No. 63/255,857, filed on Oct. 14, 2021.

(51) Int. Cl. C12N 5/0775; A61K 31/713; A61K 35/12; A61K 38/20; C12N 15/113

(52) U.S. Cl. CPC C12N 5/0662; A61K 31/713; A61K 35/12; A61K 38/2013; C12N 15/113; C12N 2310/11; C12N 2320/32; C12N 2510/02

(57) ABSTRACT

The present disclosure provides methods of producing extracellular vesicles and methods of increasing extracellular vesicle production from producer cells, which exhibit a reduced gene and/or protein function in a cholesterol biosynthetic pathway. Also provided are cell compositions having a reduced gene and/or protein function in a cholesterol biosynthetic pathway. Reducing gene and/or protein function in a cholesterol biosynthetic pathway increases the yield and production of extracellular vesicles from the producer cells.

### CROSS-REFERENCE TO RELATED APPLICATIONS

This application claims priority benefit of U.S. Provisional Application No. 63/255,857, filed Oct. 14, 2021, which is herein incorporated by reference in its entirety.

### REFERENCE TO SEQUENCE LISTING SUBMITTED ELECTRONICALLY VIA EFS-WEB

The content of the electronically submitted sequence listing in ASCII text file (Name: 4000_129PC01_Seglisting_ST26; Size: 24,490 bytes; and Date of Creation: Oct. 14, 2022) filed with the application is herein incorporated by reference in its entirety.

### FIELD OF DISCLOSURE

The present disclosure relates to modified producer cells that can be used to obtain extracellular vesicles, e.g., exosomes. The present disclosure also relates to methods of producing EVs, e.g., exosomes, from such modified producer cells.

### BACKGROUND

Extracellular vesicles, in particular exosomes, have been gaining interest as a new modality capable of an efficient delivery of various payloads to cells of all types within a living organism. As efforts accelerate to translate exosome biology into new medicines, technology gaps have emerged between the current state of the art for producing exosomes and the capabilities necessary to support large scale clinical and commercial manufacturing. To that end, considerable attempts have been focused on sustaining growth and productivity of the producer cell line in vitro, however, maximizing exosome yield remains a challenge. Therefore, novel methods for efficient, low-cost and reliable high titer production of extracellular vesicles are needed.

### SUMMARY OF DISCLOSURE

In some aspects, the present disclosure provides a method of increasing a number of extracellular vesicles (EVs) produced from producer cells, comprising modifying the producer cells to exhibit a reduced gene and/or protein function in a cholesterol biosynthetic pathway of the producer cells. In some aspects, the present disclosure also provides a method of producing extracellular vesicles (EVs) from producer cells, comprising culturing the producer cells, which exhibit a reduced gene and/or protein function in a cholesterol biosynthetic pathway. In some aspects, the reduced gene and/or protein function in a cholesterol biosynthesis comprises one or more genes selected from 3-hydroxy-3-methylglutaryl-coenzyme A reductase (HMGCR), sterol regulatory element-binding protein 2 (SREBF2), Squalene epoxidase (SQLE), or 7-Dehydrocholesterol reductase (DHCR7) or a protein encoded by the gene. In some aspects, the EVs produced by the producer cells have an increased yield compared to EVs produced by producer cells that the gene and/or protein function in a cholesterol biosynthetic pathway is not reduced. In some aspects, the yield is increased at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 16 fold, at least about 17 fold, at least about 18 fold, at least about 19 fold, at least about 20 fold, at least about 21 fold, at least about 22 fold, at least about 23 fold, at least about 24 fold, at least about 25 fold, at least about 26 fold, at least about 27 fold, at least about 28 fold, at least about 29 fold, or at least about 30 fold. In some aspects, the yield is increased about 2 fold to about 30 fold, about 2 fold to about 25 fold, about 2 fold to about 20 fold, about 2 fold to about 15 fold, about 2 fold to about 10 fold, about 2 fold to about 5 fold, about 5 fold to about 30 fold, about 5 fold to about 25 fold, about 5 fold to about 20 fold, about 5 fold to about 15 fold, about 5 fold to about 10 fold, about 10 fold to about 30 fold, about 10 fold to about 25 fold, about 10 fold to about 20 fold, about 10 fold to about 15 fold, about 15 fold to about 30 fold, about 15 fold to about 25 fold, about 15 fold to about 20 fold, about 20 fold to about 30 fold, about 20 fold to about 25 fold, or about 25 fold to about 30 fold. In some aspects, the yield is increased about 2 fold, about 3 fold, about 4 fold, about 5 fold, about 6 fold, about 7 fold, about 8 fold, about 9 fold, or about 10 fold. In some aspects, the reduced gene in a cholesterol biosynthesis is SREBF2. In some aspects, wherein the SREBF2 gene expression is reduced about 2 fold to about 20 fold. In some aspects, the SREBF2 gene expression is reduced about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, or about 20 fold. In some aspects, the reduced gene in a cholesterol biosynthesis is HMGCR. In some aspects, the HMGCR gene expression is reduced about 2 fold to about 30 fold. In some aspects, the HMGCR gene expression is reduced about 2 fold, about 3 fold, about 4 fold, about 5 fold, about 6 fold, about 7 fold, about 8 fold, about 9 fold, about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, or about 30 fold. In some aspects, the gene and/or protein function in a cholesterol biosynthetic pathway is reduced at least about 90%, at least about 80%, at least about 70%, at least about 60%, at least about 50%, at least about 40%, at least about 30%, at least about 20%, or at least about 10%, at least about 5%, or at least about 1%. In some aspects, the gene and/or protein function in a cholesterol biosynthetic pathway is reduced at least about 90%, at least about 80%, at least about 70%, at least about 60%, at least about 50%, at least about 40%, or at least about 30%. In some aspects, the modifying comprises contacting the producer cells with an agent capable of reducing the gene and/or protein function in a cholesterol biosynthetic pathway. In some aspects, the producer cells are modified prior to the culturing by contacting the producer cells with an agent capable of reducing the gene and/or protein function in a cholesterol biosynthetic pathway. In some aspects, the agent comprises a statin, a cariprazine, a PROTAC, AY9944, or BM15766. In some aspects, the statin comprises atorvastatin, lovastatin, pitavastatin, pravastatin, fluvastatin, cerivastatin, rosuvastatin, simvastatin, or combinations thereof. In some aspects, the statin is contacted at a concentration of about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, or about 20 nM, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 260 nM, about 270 nM, about 280 nM, about 290 nM, about 300 nM, about 310 nM, about 320 nM, about 330 nM, about 340 nM, about 350 nM, about 360 nM, about 370 nM, about 380 nM, about 390 nM, about 400 nM, about 410 nM, about 420 nM, about 430 nM, about 440 nM, about 450 nM, about 460 nM, about 470 nM, about 480 nM, about 490 nM, about 500 nM, about 510 nM, about 520 nM, about 530 nM, about 540 nM, about 550 nM, about 560 nM, about 570 nM, about 580 nM, about 590 nM, about 600 nM, about 610 nM, about 620 nM, about 630 nM, about 640 nM, about 650 nM, about 660 nM, about 670 nM, about 680 nM, about 690 nM, about 700 nM, about 710 nM, about 720 nM, about 730 nM, about 740 nM, about 750 nM, about 760 nM, about 770 nM, about 780 nM, about 790 nM, about 800 nM, about 810 nM, about 820 nM, about 830 nM, about 840 nM, about 850 nM, about 860 nM, about 870 nM, about 880 nM, about 890 nM, about 900 nM, about 910 nM, about 920 nM, about 930 nM, about 940 nM, about 950 nM, about 960 nM, about 970 nM, about 980 nM, about 990 nM, or about 1,000 nM. In some aspects, the statin is contacted at a concentration of between about 0.1 nM to about 100 nM, about 0.1 nM to about 90 nM, about 0.1 nM to about 80 nM, about 0.1 nM to about 70 nM, about 0.1 nM to about 60 nM, about 0.1 nM to about 50 nM, about 0.1 nM to about 40 nM, about 0.1 nM to about 30 nM, about 0.1 nM to about 20 nM, 0.1 nM to about 10 nM, or about 1 nM to about 20 nM, about 1 nM to about 10 nM, about 1 nM to about 5 nM, about 5 nM to about 20 nM, about 5 nM to about 15 nM, about 5 nM to about 10 nM, about 10 nM to about 50 nM, about 10 nM to about 40 nM, about 10 nM to about 30 nM, about 10 nM to about 20 nM, about 1 nM to about 10 nM, or about 10 nM to about 20 nM. In some aspects, the agent capable of reducing the gene and/or protein function in a cholesterol biosynthetic pathway comprises a gene editing technology. In some aspects, the gene editing technology comprises a shRNA, siRNA, miRNA, antisense oligonucleotides, CRISPR, zinc finger nuclease, TALEN, meganuclease, restriction endonuclease, or any combination thereof. In some aspects, the gene editing technology comprises siRNA. In some aspects, the EVs produced by the producer cells have decreased cholesterol content per EV compared to EVs produced by producer cells that the gene and/or protein function in a cholesterol biosynthetic pathway is not reduced. In some aspects, the cholesterol content per EV is reduced by about 10%, about 20%, about 30%, about 40%, about 50%, about 60%, about 70%, or about 80%. In some aspects, the cholesterol content per EV is reduced by about 1% to about 80%, about 10% to about 80%, about 10% to about 70%, about 10% to about 60%, about 10% to about 50%, about 10% to about 40%, about 10% to about 30%, about 10% to about 20%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 30% to about 40%, about 40% to about 80%, about 40% to about 70%, about 40% to about 60%, or about 40% to about 50%. In some aspects, the EVs produced by the producer cells do not have a difference in average size distribution compared to EVs produced by producer cells that the gene and/or protein function in a cholesterol biosynthetic pathway is not reduced. In some aspects, the producer cells are mammalian cells. In some aspects, the producer cells are HEK293 cells, HEK293S cells, HEK293SF cells, Chinese Hamster Ovary (CHO) cells, mesenchymal stem cells (MSCs), BJ human foreskin fibroblast cells, fHIDF fibroblast cells, AGE.HN® neuronal precursor cells, CAP® amniocyte cells, adipose mesenchymal stem cells, RPTEC/TERT1 cells, dendritic cells, macrophages, B cells, mast cells, neutrophils, Kupffer-Browicz cells, PER.C6 cells, Induced pluripotent stem cells (iPSCs), or C2C12 cells. In some aspects, the producer cells are stem cells. In some aspects, the EVs further comprise a scaffold moiety. In some aspects, the scaffold moiety comprises a Scaffold X. In some aspects, Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), and any combination thereof. In some aspects, the scaffold moiety is a PTGFRN protein. In some aspects, wherein the scaffold moiety comprises an amino acid sequence having at least 50%, at least 60%, at least 70%, at least 80%, at least 85%, at least 90%, at least 95%, at least 96%, at least 97%, at least 98%, at least 99%, or about 100% sequence identity to SEQ ID NO: 1. In some aspects, the scaffold moiety comprises a Scaffold Y. In some aspects, the Scaffold Y is selected from the group consisting of myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), and any combination thereof. In some aspects, the EV further comprises at least a therapeutic agent linked to a scaffold moiety. In some aspects, the EV further comprises at least a therapeutic agent. In some aspects, the therapeutic agent comprises a cytokine, a small molecule, a growth factor, an antigen, an antisense oligonucleotide, an siRNA, an shRNA, a miRNA, a dsDNA, a lncRNA, a PROTAC, an adjuvant, an immune modulator, or any combination thereof. In some aspects, the therapeutic agent is IL-12. In some aspects, the therapeutic agent is a STING agonist. In some aspects, the therapeutic agent is an antisense oligonucleotide. In some aspects, the antisense oligonucleotide targets Kras, STAT3, Nras, STAT6, CEBP/b, NLRP3, or any combination thereof.

The present disclosure also provides, in some aspects, producer cells for use in the methods described herein. In some aspects, the present disclosure also provides producer cells prepared by the methods described herein.

In some aspects, the present disclosure also provides EVs produced by the methods described herein.

In some aspects, the present disclosure also provides a bioreactor comprising the producer cells or EVs described herein.

In some aspects, the present disclosure also provides a method of treating or preventing a disease or a condition in a subject in need thereof comprising administering the extracellular vesicles described herein.

In some aspects, the present disclosure provides use of the extracellular vesicles described herein to treat or prevent a disease or condition in a subject in need thereof.

In some aspects, the present disclosure provides extracellular vesicles described herein for treating or preventing a disease or condition in a subject in need thereof.

### BRIEF DESCRIPTION OF FIGURES

FIG. 1 shows siRNA knock down of two genes that are key regulators in the cholesterol synthesis pathway (e.g., HMGCR and SREBF2) significantly increase relative luminescence normalized per cell to the untreated control, corresponding to increased EV production per cell.

FIG. 2A shows cell viability (%) and FIG. 2B shows total cell density for untreated, non-target siRNA treated, PTGFRN siRNA treated, SREBF2 siRNA treated, and HMGCR siRNA treated producer cells. FIG. 2C shows density gradients of EVs harvested from the producer cells of FIGS. 2A and 2B. FIG. 2D shows a SDS-PAGE gel of the protein contents of EVs harvested from control, SREBF2 knock down, and HMGCR knock down producer cells. FIG. 2E shows the total amount of EVs produced per cell in untreated, non-target siRNA treated, PTGFRN siRNA treated, SREBF2 siRNA treated, and HMGCR siRNA treated producer cells. FIG. 2F shows the fold increase in purified EVs from untreated, non-target siRNA treated, PTGFRN siRNA treated, SREBF2 siRNA treated, and HMGCR siRNA treated producer cells. FIG. 2G shows total cholesterol (μg/mL) measured by Amplex™ Red in EVs purified from untreated, non-target siRNA treated, PTGFRN siRNA treated, SREBF2 siRNA treated, and HMGCR siRNA treated producer cells. FIG. 2H shows the total amount of purified EVs measured by nanoparticle tracking analysis (NTA) in untreated, non-target siRNA treated, PTGFRN siRNA treated, SREBF2 siRNA treated, and HMGCR siRNA treated producer cells. FIG. 2I shows the fold change in cholesterol content of purified EVs from untreated, non-target siRNA treated, PTGFRN siRNA treated, SREBF2 siRNA treated, and HMGCR siRNA treated producer cells. FIG. 2J shows the average diameter of EVs purified from untreated, non-target siRNA treated, PTGFRN siRNA treated, SREBF2 siRNA treated, and HMGCR siRNA treated producer cells.

FIGS. 3A-3D show the effect of pharmacological inhibition of cholesterol biosynthesis on EV titer. FIG. 3A shows cell viability of simvastatin treatment from Day 1 to Day 6 (control, 10 nM, 50 nM, 250 nM, 1 μM, 10 μM, and 50 μM). FIG. 3B shows the viable cell density of simvastatin treatment from Day 1 to Day 6 (control, 10 nM, 50 nM, 250 nM, 1 μM, 10 μM, and 50 μM). FIG. 3C shows viability of cariprazine treated producer cells on Days 1, 2, and 3 of treatment (control, 2 μM, 5 μM, and 10 μM). FIG. 3D shows viable cell density of cariprazine treatment from Day 1 to Day 3 (control, 2 μM, 5 μM, and 10 μM).

FIGS. 4A-4C show the viable cell density (FIG. 4A), viability (FIG. 4B), and HiBit luciferase for simvastatin treated HiBit report cell lines (FIG. 4C). FIG. 4D shows the effect of simvastatin (SIM) treatment on EV titer from the HiBit luciferase reporter assay. Fold change to untreated control is shown as a function of days in culture and simvastatin concentrations (2 nM, 5 nM, and 10 nM). FIG. 4E shows the effect of cariprazine (CAR) treatment on EV titer from the HiBit luciferase reporter assay. Fold change to untreated control is shown as a function of days in culture and cariprazine concentration (5 μM and 10 μM).

FIG. 5A shows a density gradient for EVs produced from Protein X (PrX) producer cells (untreated and 5 nM simvastatin) and a density gradient for EVs produced from exoIL-12 producer cells (untreated, 5 nM simvastatin, 10 nM simvastatin). FIG. 5B displays protein gels of the EVs harvested in FIG. 5A.

FIGS. 6A and 6B show results from an in vitro HEK IL-12 reporter assay for EVs (exoIL-12) produced from simvastatin treated producer cells. FIG. 6A shows in vitro potency in a HEK IL-12 reporter assay among recombinant IL-12, exoIL-12 drug product, EVs (exoIL-12) from untreated, 5 nM, and 10 nM simvastatin (SM) treated producer cells. FIG. 6B shows in vitro potency in a HEK IL-12 reporter assay for exo-IL-12 drug product, and exoIL-12 from control, 5 nM simvastatin (SM), and 10 nM simvastatin (SM) treated producer cells.

FIG. 7 is a bar graph showing results from an in vitro assay for the induction of exosome production by producer cells and the effect of culture with 10 nM, 25, nM, 50 nM, 75 nM, and 100 nM of the cholesterol-synthesis inhibitor rosuvastatin (ROS) on HiBit reporter cells. Data are presented as luminescence/cell.

FIGS. 8A and 8B demonstrate the lack of involvement of prenylation in exosome production. FIGS. 8A and 8B are graphical representations of the results from HiBit reporter cells exposed to the GGTase inhibitor GGTI2418. FIG. 8A shows viable cell density (VCD; as 10⁶) as a function of days in culture. FIG. 8B shows luminescence (RLU) as a function of days in culture.

FIGS. 9A and 9B are line graphs showing the effects on viable cell density (VCD; FIG. 9A) and exosome production (FIG. 9B) by adding exogenous cholesterol to producer cells cultured in simvastatin.

### DETAILED DESCRIPTION OF DISCLOSURE

The present disclosure is directed to methods of increasing a number of extracellular vesicles (EVs) produced from producer cells, comprising modifying the producer cells to exhibit a reduced gene and/or protein function in a cholesterol biosynthetic pathway of the producer cells. The present disclosure is also directed methods of producing extracellular vesicles (EVs) from producer cells, comprising culturing the producer cells, which exhibit a reduced gene and/or protein function in a cholesterol biosynthetic pathway.

Before the present disclosure is described in greater detail, it is to be understood that this disclosure is not limited to the particular compositions or process steps described, as such can, of course, vary. As will be apparent to those of skill in the art upon reading this disclosure, each of the individual aspects described and illustrated herein has discrete components and features which can be readily separated from or combined with the features of any of the other several aspects without departing from the scope or spirit of the present disclosure. Any recited method can be carried out in the order of events recited or in any other order which is logically possible.

The headings provided herein are not limitations of the various aspects of the disclosure, which can be defined by reference to the specification as a whole. It is also to be understood that the terminology used herein is for the purpose of describing particular aspects only, and is not intended to be limiting, since the scope of the present disclosure will be limited only by the appended claims.

### I. Definitions

In order that the present description can be more readily understood, certain terms are first defined. Additional definitions are set forth throughout the detailed description.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein. It is further noted that the claims can be drafted to exclude any optional element. As such, this statement is intended to serve as antecedent basis for use of such exclusive terminology as “solely,” “only” and the like in connection with the recitation of claim elements, or use of a negative limitation.

Furthermore, “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Where a range of values is recited, it is to be understood that each intervening integer value, and each fraction thereof, between the recited upper and lower limits of that range is also specifically disclosed, along with each subrange between such values. The upper and lower limits of any range can independently be included in or excluded from the range, and each range where either, neither or both limits are included is also encompassed within the disclosure. Thus, ranges recited herein are understood to be shorthand for all of the values within the range, inclusive of the recited endpoints. For example, a range of 1 to 10 is understood to include any number, combination of numbers, or sub-range from the group consisting of 1, 2, 3, 4, 5, 6, 7, 8, 9, and 10

Where a value is explicitly recited, it is to be understood that values which are about the same quantity or amount as the recited value are also within the scope of the disclosure. Where a combination is disclosed, each subcombination of the elements of that combination is also specifically disclosed and is within the scope of the disclosure. Conversely, where different elements or groups of elements are individually disclosed, combinations thereof are also disclosed. Where any element of a disclosure is disclosed as having a plurality of alternatives, examples of that disclosure in which each alternative is excluded singly or in any combination with the other alternatives are also hereby disclosed; more than one element of a disclosure can have such exclusions, and all combinations of elements having such exclusions are hereby disclosed.

Nucleotides are referred to by their commonly accepted single-letter codes. Unless otherwise indicated, nucleotide sequences are written left to right in 5′ to 3′ orientation. Nucleotides are referred to herein by their commonly known one-letter symbols recommended by the IUPAC-IUB Biochemical Nomenclature Commission. Accordingly, A represents adenine, C represents cytosine, G represents guanine, T represents thymine, and U represents uracil.

Amino acid sequences are written left to right in amino to carboxy orientation. Amino acids are referred to herein by either their commonly known three letter symbols or by the one-letter symbols recommended by the IUPAC-IUB Biochemical Nomenclature Commission.

The headings provided herein are not limitations of the various aspects of the disclosure, which can be had by reference to the specification as a whole. Accordingly, the terms defined immediately below are more fully defined by reference to the specification in its entirety.

The term “about” or “approximately” is used herein to mean approximately, roughly, around, or in the region of. When the term “about” is used in conjunction with a numerical range, it modifies that range by extending the boundaries above and below the numerical values set forth. In general, the term “about” can modify a numerical value above and below the stated value by a variance of, e.g., 10 percent, up or down (higher or lower). In some aspects, the term used herein means within 5% of the referenced amount, e.g., about 50% is understood to encompass a range of values from 47.5% to 52.5%.

As used herein, the term “extracellular vesicle” or “EV” refers to a cell-derived vesicle comprising a membrane that encloses an internal space. Extracellular vesicles comprise all membrane-bound vesicles (e.g., exosomes, nanovesicles) that have a smaller diameter than the cell from which they are derived. Generally, extracellular vesicles range in diameter from 20 nm to 1000 nm, and can comprise various macromolecular payload either within the internal space (i.e., lumen), displayed on the external surface of the extracellular vesicle, and/or spanning the membrane. In some aspects, the payload can comprise nucleic acids, proteins, carbohydrates, lipids, small molecules, and/or combinations thereof. In certain aspects, an extracellular vesicle comprises a scaffold moiety. By way of example and without limitation, extracellular vesicles include apoptotic bodies, fragments of cells, vesicles derived from cells by direct or indirect manipulation (e.g., by serial extrusion or treatment with alkaline solutions), vesiculated organelles, and vesicles produced by living cells (e.g., by direct plasma membrane budding or fusion of the late endosome with the plasma membrane). Extracellular vesicles can be derived from a living or dead organism, explanted tissues or organs, prokaryotic or eukaryotic cells, and/or cultured cells. In some aspects, the extracellular vesicles are produced by cells that express one or more transgene products.

As used herein the term “exosome” refers to a cell-derived small (between 20-300 nm in diameter, more preferably 40-200 nm in diameter) vesicle comprising a membrane that encloses an internal space (i.e., lumen), and which is generated from said cell by direct plasma membrane budding or by fusion of the late endosome with the plasma membrane. The exosome is a species of extracellular vesicle. The exosome comprises lipid or fatty acid and polypeptide and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. In some aspects, an exosome comprises a scaffold moiety. The exosome can be derived from a producer cell, and isolated from the producer cell based on its size, density, biochemical parameters, or a combination thereof. In some aspects, the exosomes of the present disclosure are produced by cells that express one or more transgene products.

As used herein, the term “nanovesicle” refers to a cell-derived small (between 20-250 nm in diameter, more preferably 30-150 nm in diameter) vesicle comprising a membrane that encloses an internal space, and which is generated from said cell by direct or indirect manipulation such that said nanovesicle would not be produced by said producer cell without said manipulation. Appropriate manipulations of said producer cell include but are not limited to serial extrusion, treatment with alkaline solutions, sonication, or combinations thereof. The production of nanovesicles may, in some instances, result in the destruction of said producer cell. Preferably, populations of nanovesicles are substantially free of vesicles that are derived from producer cells by way of direct budding from the plasma membrane or fusion of the late endosome with the plasma membrane. The nanovesicle comprises lipid or fatty acid and polypeptide, and optionally comprises a payload (e.g., a therapeutic agent), a receiver (e.g., a targeting moiety), a polynucleotide (e.g., a nucleic acid, RNA, or DNA), a sugar (e.g., a simple sugar, polysaccharide, or glycan) or other molecules. In some aspects, a nanovesicle comprises a scaffold moiety. The nanovesicle, once it is derived from a producer cell according to said manipulation, may be isolated from the producer cell based on its size, density, biochemical parameters, or a combination thereof.

As used herein the term “surface-engineered EVs, e.g., exosomes” (e.g., Scaffold X-engineered EVs, e.g., exosomes) refers to an EV, e.g., exosome, with the membrane or the surface of the EV, e.g., exosome, modified in its composition so that the surface of the engineered EV, e.g., exosome, is different from that of the EV, e.g., exosome, prior to the modification or of the naturally occurring EV, e.g., exosome. The engineering can be on the surface of the EV, e.g., exosome, or in the membrane of the EV, e.g., exosome, so that the surface of the EV, e.g., exosome, is changed. For example, the membrane is modified in its composition of a protein, a lipid, a small molecule, a carbohydrate, etc. The composition can be changed by a chemical, a physical, or a biological method or by being produced from a cell previously or concurrently modified by a chemical, a physical, or a biological method. Specifically, the composition can be changed by a genetic engineering or by being produced from a cell previously modified by genetic engineering. In some aspects, a surface-engineered EV, e.g., exosome, comprises an exogenous protein (i.e., a protein that the EV, e.g., exosome, does not naturally express) or a fragment or variant thereof that can be exposed to the surface of the EV, e.g., exosome, or can be an anchoring point (attachment) for a moiety exposed on the surface of the EV, e.g., exosome. In other aspects, a surface-engineered EV, e.g., exosome, comprises a higher expression (e.g., higher number) of a natural exosome protein (e.g., Scaffold X) or a fragment or variant thereof that can be exposed to the surface of the EV, e.g., exosome, or can be an anchoring point (attachment) for a moiety exposed on the surface of the EV, e.g., exosome.

As used herein the term “lumen-engineered exosome” (e.g., Scaffold Y-engineered exosome) refers to an EV, e.g., exosome, with the membrane or the lumen of the EV, e.g., exosome, modified in its composition so that the lumen of the engineered EV, e.g., exosome, is different from that of the EV, e.g., exosome, prior to the modification or of the naturally occurring EV, e.g., exosome. The engineering can be directly in the lumen or in the membrane of the EV, e.g., exosome so that the lumen of the EV, e.g., exosome is changed. For example, the membrane is modified in its composition of a protein, a lipid, a small molecule, a carbohydrate, etc. so that the lumen of the EV, e.g., exosome is modified. The composition can be changed by a chemical, a physical, or a biological method or by being produced from a cell previously modified by a chemical, a physical, or a biological method. Specifically, the composition can be changed by a genetic engineering or by being produced from a cell previously modified by genetic engineering. In some aspects, a lumen-engineered exosome comprises an exogenous protein (i.e., a protein that the EV, e.g., exosome does not naturally express) or a fragment or variant thereof that can be exposed in the lumen of the EV, e.g., exosome or can be an anchoring point (attachment) for a moiety exposed on the inner layer of the EV, e.g., exosome. In other aspects, a lumen-engineered EV, e.g., exosome, comprises a higher expression of a natural exosome protein (e.g., Scaffold X or Scaffold Y) or a fragment or variant thereof that can be exposed to the lumen of the exosome or can be an anchoring point (attachment) for a moiety exposed in the lumen of the exosome.

The term “modified,” when used in the context of EVs, e.g., exosomes described herein, refers to an alteration or engineering of an EV, e.g., exosome and/or its producer cell, such that the modified EV, e.g., exosome is different from a naturally-occurring EV, e.g., exosome. In some aspects, a modified EV, e.g., exosome described herein comprises a membrane that differs in composition of a protein, a lipid, a small molecular, a carbohydrate, etc. compared to the membrane of a naturally-occurring EV, e.g., exosome (e.g., membrane comprises higher density or number of natural exosome proteins and/or membrane comprises proteins that are not naturally found in exosomes (e.g., an IL-12 moiety). In certain aspects, such modifications to the membrane changes the exterior surface of the EV, e.g., exosome (e.g., surface-engineered EVs, e.g., exosomes described herein). In certain aspects, such modifications to the membrane changes the lumen of the EV, e.g., exosome (e.g., lumen-engineered EVs, e.g., exosomes described herein).

As used herein, the term “scaffold moiety” refers to a molecule that can be used to anchor an IL-12 moiety or any other compound of interest to the EV, e.g., exosome, either on the luminal surface or on the exterior surface of the EV, e.g., exosome. In certain aspects, a scaffold moiety comprises a synthetic molecule. In some aspects, a scaffold moiety comprises a non-polypeptide moiety. In other aspects, a scaffold moiety comprises a lipid, carbohydrate, or protein that naturally exists in the EV, e.g., exosome. In some aspects, a scaffold moiety comprises a lipid, carbohydrate, or protein that does not naturally exist in the EV, e.g., exosome. In certain aspects, a scaffold moiety is Scaffold X. In some aspects, a scaffold moiety is Scaffold Y. In further aspects, a scaffold moiety comprises both Scaffold X and Scaffold Y. In certain aspects, a scaffold moiety comprises Lamp-1, Lamp-2, CD13, CD86, Flotillin, Syntaxin-3, CD2, CD36, CD40, CD40L, CD41a, CD44, CD45, ICAM-1, Integrin alpha4, LiCAM, LFA-1, Mac-1 alpha and beta, Vti-1A and B, CD3 epsilon and zeta, CD9, CD18, CD37, CD53, CD63, CD81, CD82, CXCR4, FcR, GluR2/3, HLA-DM (MHC II), immunoglobulins, MHC-I or MHC-II components, TCR beta, tetraspanins, or combinations thereof.

As used herein, the term “Scaffold X” refers to exosome proteins that have recently been identified on the surface of exosomes. See, e.g., U.S. Pat. No. 10,195,290, which is incorporated herein by reference in its entirety. Non-limiting examples of Scaffold X proteins include: prostaglandin F2 receptor negative regulator (“the PTGFRN protein”); basigin (“the BSG protein”); immunoglobulin superfamily member 2 (“the IGSF2 protein”); immunoglobulin superfamily member 3 (“the IGSF3 protein”); immunoglobulin superfamily member 8 (“the IGSF8 protein”); integrin beta-1 (“the ITGB1 protein); integrin alpha-4 (“the ITGA4 protein”); 4F2 cell-surface antigen heavy chain (“the SLC3A2 protein”); and a class of ATP transporter proteins (“the ATP1A1 protein,” “the ATP1A2 protein,” “the ATP1A3 protein,” “the ATP1A4 protein,” “the ATP1B3 protein,” “the ATP2B1 protein,” “the ATP2B2 protein,” “the ATP2B3 protein,” “the ATP2B protein”). In some aspects, a Scaffold X protein can be a whole protein or a fragment thereof (e.g., functional fragment, e.g., the smallest fragment that is capable of anchoring another moiety on the exterior surface or on the luminal surface of the EV, e.g., exosome). In some aspects, a Scaffold X can anchor a moiety (e.g., a therapeutic protein, e.g., an IL-12 moiety) to the external surface or the luminal surface of the exosome.

As used herein, the term “Scaffold Y” refers to exosome proteins that were newly identified within the luminal surface of exosomes. See, e.g., International Publication. No. WO/2019/099942, which is incorporated herein by reference in its entirety. Non-limiting examples of Scaffold Y proteins include: myristoylated alanine rich Protein Kinase C substrate (“the MARCKS protein”); myristoylated alanine rich Protein Kinase C substrate like 1 (“the MARCKSL1 protein”); and brain acid soluble protein 1 (“the BASP1 protein”). In some aspects, a Scaffold Y protein can be a whole protein or a fragment thereof (e.g., functional fragment, e.g., the smallest fragment that is capable of anchoring a moiety on the luminal surface of the EVs, e.g., exosomes). In some aspects, a Scaffold Y can anchor an IL-2 moiety to the lumen of the EVs, e.g., exosomes.

As used herein, the term “fragment” of a protein (e.g., therapeutic protein, Scaffold X, or Scaffold Y) refers to an amino acid sequence of a protein that is shorter than the naturally-occurring sequence, N- and/or C-terminally deleted or any part of the protein deleted in comparison to the naturally occurring protein. As used herein, the term “functional fragment” refers to a protein fragment that retains protein function. Accordingly, in some aspects, a functional fragment of a Scaffold X protein retains the ability to anchor a moiety on the luminal surface or on the exterior surface of the EV, e.g., exosome. Similarly, in certain aspects, a functional fragment of a Scaffold Y protein retains the ability to anchor a moiety on the luminal surface of the EV, e.g., exosome. Whether a fragment is a functional fragment can be assessed by any art known methods to determine the protein content of EVs, e.g., exosomes including Western Blots, FACS analysis and fusions of the fragments with autofluorescent proteins like, e.g., GFP. In certain aspects, a functional fragment of a Scaffold X protein retains at least about 50%, at least about 60%, at least about 70%, at least about 80%, at least about 90% or at least about 100% of the ability, e.g., an ability to anchor a moiety, of the naturally occurring Scaffold X protein. In some aspects, a functional fragment of a Scaffold Y protein retains at least about 50%, at least about 60%, at least about 70%, at least about 80%, at least about 90% or at least about 100% of the ability, e.g., an ability to anchor another molecule, of the naturally occurring Scaffold Y protein.

As used herein, the term “variant” of a molecule (e.g., functional molecule, antigen, Scaffold X and/or Scaffold Y) refers to a molecule that shares certain structural and functional identities with another molecule upon comparison by a method known in the art. For example, a variant of a protein can include a substitution, insertion, deletion, frameshift or rearrangement in another protein.

In some aspects, a variant of a Scaffold X comprises a variant having at least about 70% identity to the full-length, mature PTGFRN, BSG, IGSF2, IGSF3, IGSF8, ITGB1, ITGA4, SLC3A2, or ATP transporter proteins or a fragment (e.g., functional fragment) of the PTGFRN, BSG, IGSF2, IGSF3, IGSF8, ITGB1, ITGA4, SLC3A2, or ATP transporter proteins. In some aspects, variants or variants of fragments of PTGFRN share at least about 70%, at least about 80%, at least about 85%, at least about 90%, at least about 95%, at least about 96%, at least about 97%, at least about 98%, or at least about 99% sequence identity with PTGFRN according to SEQ ID NO: 1 or with a functional fragment thereof.

In some aspects, a variant of a Scaffold Y comprises a variant having at least 70% identity to MARCKS, MARCKSL1, BASP1, or a fragment of MARCKS, MARCKSL1, or BASP1. In some aspects variants or variants of fragments of MARCKS share at least about 70%, at least about 80%, at least about 85%, at least about 90%, at least about 95%, at least about 96%, at least about 97%, at least about 98%, or at least about 99% sequence identity with MARCKS according to SEQ ID NO: 47 or with a functional fragment thereof. In some aspects variants or variants of fragments of MARCKSL1 share at least about 70%, at least about 80%, at least about 85%, at least about 90%, at least about 95%, at least about 96%, at least about 97%, at least about 98%, or at least about 99% sequence identity with MARCKSL1 according to SEQ ID NO: 48 or with a functional fragment thereof. In some aspects variants or variants of fragments of BASP1 share at least about 70%, at least about 80%, at least about 85%, at least about 90%, at least about 95%, at least about 96%, at least about 97%, at least about 98%, or at least about 99% sequence identity with BASP1 according to SEQ ID NO: 49 or with a functional fragment thereof. In some aspects, the variant or variant of a fragment of Scaffold Y protein retains the ability to be specifically targeted to the lumen of EVs, e.g., exosomes. In some aspects, the Scaffold Y includes one or more mutations, e.g., conservative amino acid substitutions.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, if an amino acid in a polypeptide is replaced with another amino acid from the same side chain family, the substitution is considered to be conservative. In another aspect, a string of amino acids can be conservatively replaced with a structurally similar string that differs in order and/or composition of side chain family members.

The term “percent sequence identity” or “percent identity” between two polynucleotide or polypeptide sequences refers to the number of identical matched positions shared by the sequences over a comparison window, taking into account additions or deletions (i.e., gaps) that must be introduced for optimal alignment of the two sequences. A matched position is any position where an identical nucleotide or amino acid is presented in both the target and reference sequence. Gaps presented in the target sequence are not counted since gaps are not nucleotides or amino acids. Likewise, gaps presented in the reference sequence are not counted since target sequence nucleotides or amino acids are counted, not nucleotides or amino acids from the reference sequence.

The percentage of sequence identity is calculated by determining the number of positions at which the identical amino-acid residue or nucleic acid base occurs in both sequences to yield the number of matched positions, dividing the number of matched positions by the total number of positions in the window of comparison and multiplying the result by 100 to yield the percentage of sequence identity. The comparison of sequences and determination of percent sequence identity between two sequences may be accomplished using readily available software both for online use and for download. Suitable software programs are available from various sources, and for alignment of both protein and nucleotide sequences. One suitable program to determine percent sequence identity is bl2seq, part of the BLAST suite of programs available from the U.S. government's National Center for Biotechnology Information BLAST web site (blast.ncbi.nlm.nih.gov). B12seq performs a comparison between two sequences using either the BLASTN or BLASTP algorithm. BLASTN is used to compare nucleic acid sequences, while BLASTP is used to compare amino acid sequences. Other suitable programs are, e.g., Needle, Stretcher, Water, or Matcher, part of the EMBOSS suite of bioinformatics programs and also available from the European Bioinformatics Institute (EBI) at www.ebi.ac.uk/Tools/psa.

Different regions within a single polynucleotide or polypeptide target sequence that aligns with a polynucleotide or polypeptide reference sequence can each have their own percent sequence identity. It is noted that the percent sequence identity value is rounded to the nearest tenth. For example, 80.11, 80.12, 80.13, and 80.14 are rounded down to 80.1, while 80.15, 80.16, 80.17, 80.18, and 80.19 are rounded up to 80.2. It also is noted that the length value will always be an integer.

One skilled in the art will appreciate that the generation of a sequence alignment for the calculation of a percent sequence identity is not limited to binary sequence-sequence comparisons exclusively driven by primary sequence data. Sequence alignments can be derived from multiple sequence alignments. One suitable program to generate multiple sequence alignments is ClustalW2, available from www.clustal.org. Another suitable program is MUSCLE, available from www.drive5.com/muscle/. ClustalW2 and MUSCLE are alternatively available, e.g., from the EBI.

It will also be appreciated that sequence alignments can be generated by integrating sequence data with data from heterogeneous sources such as structural data (e.g., crystallographic protein structures), functional data (e.g., location of mutations), or phylogenetic data. A suitable program that integrates heterogeneous data to generate a multiple sequence alignment is T-Coffee, available at www.tcoffee.org, and alternatively available, e.g., from the EBI. It will also be appreciated that the final alignment used to calculate percent sequence identity may be curated either automatically or manually.

The polynucleotide variants can contain alterations in the coding regions, non-coding regions, or both. In one aspect, the polynucleotide variants contain alterations which produce silent substitutions, additions, or deletions, but do not alter the properties or activities of the encoded polypeptide. In another aspect, nucleotide variants are produced by silent substitutions due to the degeneracy of the genetic code. In other aspects, variants in which 5-10, 1-5, or 1-2 amino acids are substituted, deleted, or added in any combination. Polynucleotide variants can be produced for a variety of reasons, e.g., to optimize codon expression for a particular host (change codons in the human mRNA to others, e.g., a bacterial host such as E. coli).

Naturally occurring variants are called “allelic variants,” and refer to one of several alternate forms of a gene occupying a given locus on a chromosome of an organism (Genes II, Lewin, B., ed., John Wiley & Sons, New York (1985)). These allelic variants can vary at either the polynucleotide and/or polypeptide level and are included in the present disclosure. Alternatively, non-naturally occurring variants can be produced by mutagenesis techniques or by direct synthesis.

Using known methods of protein engineering and recombinant DNA technology, variants can be generated to improve or alter the characteristics of the polypeptides. For instance, one or more amino acids can be deleted from the N-terminus or C-terminus of the secreted protein without substantial loss of biological function. Ron et al., J. Biol. Chem. 268: 2984-2988 (1993), incorporated herein by reference in its entirety, reported variant KGF proteins having heparin binding activity even after deleting 3, 8, or 27 amino-terminal amino acid residues. Similarly, interferon gamma exhibited up to ten times higher activity after deleting 8-10 amino acid residues from the carboxy terminus of this protein. (Dobeli et al., J. Biotechnology 7:199-216 (1988), incorporated herein by reference in its entirety.)

Moreover, ample evidence demonstrates that variants often retain a biological activity similar to that of the naturally occurring protein. For example, Gayle and coworkers (J. Biol. Chem 268:22105-22111 (1993), incorporated herein by reference in its entirety) conducted extensive mutational analysis of human cytokine IL-la. They used random mutagenesis to generate over 3,500 individual IL-la mutants that averaged 2.5 amino acid changes per variant over the entire length of the molecule. Multiple mutations were examined at every possible amino acid position. The investigators found that “[m]ost of the molecule could be altered with little effect on either [binding or biological activity].” (See Abstract.) In fact, only 23 unique amino acid sequences, out of more than 3,500 nucleotide sequences examined, produced a protein that significantly differed in activity from wild-type.

As stated above, polypeptide variants include, e.g., modified polypeptides. Modifications include, e.g., acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation (Mei et al., Blood 116:270-79 (2010), which is incorporated herein by reference in its entirety), proteolytic processing, phosphorylation, prenylation, racemization, selenoylation, sulfation, transfer-RNA mediated addition of amino acids to proteins such as arginylation, and ubiquitination. In some aspects, Scaffold X and/or Scaffold Y is modified at any convenient location.

As used herein the term “linked to” or “conjugated to” are used interchangeably and refer to a covalent or non-covalent bond formed between a first moiety and a second moiety, e.g., Scaffold X and an IL-12 moiety, respectively, e.g., a scaffold moiety expressed in or on the extracellular vesicle and an IL-12 moiety, e.g., Scaffold X (e.g., a PTGFRN protein), respectively, in the luminal surface of or on the external surface of the extracellular vesicle.

The term “encapsulated”, or grammatically different forms of the term (e.g., encapsulation, or encapsulating) refers to a status or process of having a first moiety (e.g., an antigen) inside a second moiety (e.g., an EV, e.g., exosome) without chemically or physically linking the two moieties. In some aspects, the term “encapsulated” can be used interchangeably with “in the lumen of”. Non-limiting examples of encapsulating a first moiety (e.g., an antigen) into a second moiety (e.g., EVs, e.g., exosomes) are disclosed elsewhere herein.

As used herein, the term “producer cell” refers to a cell used for generating an EV, e.g., exosome. A producer cell can be a cell cultured in vitro, or a cell in vivo. A producer cell includes, but not limited to, a cell known to be effective in generating EVs, e.g., exosomes, e.g., HEK293 cells, C2C12 cells, Chinese hamster ovary (CHO) cells, mesenchymal stem cells (MSCs), BJ human foreskin fibroblast cells, s9f cells, fHDF fibroblast cells, AGE.HN® neuronal precursor cells, CAP® amniocyte cells, adipose mesenchymal stem cells, and RPTEC/TERT1 cells. In certain aspects, a producer cell is an antigen-presenting cell. In some aspects, the producer cell is a bacterial cell. In some aspects, a producer cell is a dendritic cell, a B cell, a mast cell, a macrophage, a neutrophil, a Kupffer-Browicz cell, or a cell derived from any of these cells, or any combination thereof. In some aspects, the producer cell is not a bacterial cell. In other aspects, the producer cell is not an antigen-presenting cell.

As used herein the term “associated with” refers to a covalent or non-covalent bond formed between a first moiety, e.g., a protein, e.g., an IL-12 moiety, and a second moiety, e.g., an extracellular vesicle, respectively; or encapsulation of a first moiety, e.g., a protein, e.g., an IL-12 moiety, into a second moiety, e.g., extracellular vesicle. For example, in some aspects, a scaffold moiety, e.g., Scaffold X (e.g., a PTGFRN protein), is expressed in or on the extracellular vesicle and a protein, e.g., an IL-12 moiety, is loaded on the external surface of the extracellular vesicle. In one aspect, the term “associated with” means a covalent, non-peptide bond or a non-covalent bond. For example, the amino acid cysteine comprises a thiol group that can form a disulfide bond or bridge with a thiol group on a second cysteine residue. Examples of covalent bonds include, but are not limited to, a peptide bond, a metal bond, a hydrogen bond, a disulfide bond, a sigma bond, a pi bond, a delta bond, a glycosidic bond, an agnostic bond, a bent bond, a dipolar bond, a Pi backbond, a double bond, a triple bond, a quadruple bond, a quintuple bond, a sextuple bond, conjugation, hyperconjugation, aromaticity, hapticity, or antibonding. Non-limiting examples of non-covalent bond include an ionic bond (e.g., cation-pi bond or salt bond), a metal bond, a hydrogen bond (e.g., dihydrogen bond, dihydrogen complex, low-barrier hydrogen bond, or symmetric hydrogen bond), van der Walls force, London dispersion force, a mechanical bond, a halogen bond, aurophilicity, intercalation, stacking, entropic force, or chemical polarity. In other aspects, the term “associated with” means that a first moiety, e.g., extracellular vesicle, encapsulates a second moiety, e.g., a protein, e.g., an IL-12 moiety. In some aspects, the first moiety and the second moiety can be linked to each other. In other aspects, the first moiety and the second moiety are not physically and/or chemically linked to each other.

The term “loaded”, or grammatically different forms of the term (e.g., load or loaded), as used herein, refers to a status or process of having a first moiety (e.g., an IL-12 moiety, a STING agonist) associated with a second moiety (e.g., an EV, e.g., and exosome). In some aspects, the first moiety is chemically or physically linked to the second moiety. In some aspects, the first moiety is not chemically or physically linked to the second moiety. In some aspects, the first moiety is present within the second moiety, e.g., within the lumen of an EV (e.g., an exosome), e.g., “encapsulated”. In some aspects, the first moiety is associated with the exterior surface of the second moiety, e.g., linked or conjugated to the surface of an EV (e.g., an exosome), e.g., “surface-display” of the second moiety.

As used herein, the terms “isolate,” “isolated,” and “isolating” or “purify,” “purified,” and “purifying” as well as “extracted” and “extracting” are used interchangeably and refer to the state of a preparation (e.g., a plurality of known or unknown amount and/or concentration) of desired EVs, that have undergone one or more processes of purification, e.g., a selection or an enrichment of the desired EV preparation. In some aspects, isolating or purifying as used herein is the process of removing, partially removing (e.g., a fraction) of the EVs from a sample containing producer cells. In some aspects, an isolated EV composition has no detectable undesired activity or, alternatively, the level or amount of the undesired activity is at or below an acceptable level or amount. In other aspects, an isolated EV composition has an amount and/or concentration of desired EVs at or above an acceptable amount and/or concentration. In other aspects, the isolated EV composition is enriched as compared to the starting material (e.g., producer cell preparations) from which the composition is obtained. This enrichment can be by 10%, 20%, 30%, 40%, 50%, 60%, 70%, 80%, 90%, 95%, 96%, 97%, 98%, 99%, 99.9%, 99.99%, 99.999%, 99.9999%, or greater than 99.9999% as compared to the starting material. In some aspects, isolated EV preparations are substantially free of residual biological products. In some aspects, the isolated EV preparations are 100% free, 99% free, 98% free, 97% free, 96% free, 95% free, 94% free, 93% free, 92% free, 91% free, or 90% free of any contaminating biological matter. Residual biological products can include abiotic materials (including chemicals) or unwanted nucleic acids, proteins, lipids, or metabolites. Substantially free of residual biological products can also mean that the EV composition contains no detectable producer cells and that only EVs are detectable.

The term “free IL-12 moiety” as used herein means an IL-12 moiety that is not associated with an extracellular vesicle, but otherwise identical to the IL-12 moiety associated with the extracellular vesicle. Especially when compared to an extracellular vesicle associated with an IL-12 moiety, the free IL-12 moiety is the same IL-12 moiety associated with the extracellular vesicle. In some aspects, when a free IL-12 moiety is compared to an extracellular vesicle comprising the IL-12 moiety in its efficacy, toxicity, and/or any other characteristics, the amount of the free IL-12 moiety compared to the IL-12 moiety associated with the extracellular vesicle is the same as the amount of the IL-12 moiety associated with the EV.

The term “exoIL-12” as used herein refers to an exosome loaded with an IL-12 moiety, e.g., an IL-12 protein or a fragment thereof. In some aspects, the IL-12 moiety is associated with the exterior surface of the exosome (e.g., surface display of the IL-12 moiety). In some aspects, the IL-12 moiety is linked to or conjugated to the exterior surface of the exosome. In some aspects, the IL-12 moiety is linked to or conjugated to a surface exposed scaffold protein, e.g, a Scaffold X protein, e.g., a PTGFRN protein. In some aspects, the IL-12 moiety is linked to or conjugated to the lipid bilayer of the exosome. In some aspects, the exosome comprises an IL-12 moiety in the lumen of the exosome. In some aspects, the IL-12 moiety is associated with the luminal surface of the exosome, e.g., with a Scaffold protein, e.g., Scaffold X, e.g., PTGFRN. In some aspects, the IL-12 moiety is encapsulated within the lumen of the exosome and is not associated with a scaffold protein.

As used herein, the term “ligand” refers to a molecule that binds to a receptor and modulates the receptor to produce a biological response. Modulation can be activation, deactivation, blocking, or damping of the biological response mediated by the receptor. Receptors can be modulated by either an endogenous or an exogenous ligand. Non-limiting examples of endogenous ligands include antibodies and peptides. Non-limiting examples of exogenous agonist include drugs, small molecules, and cyclic dinucleotides. The ligand can be a full, partial, or inverse ligand.

As used herein, the term “pharmaceutical composition” refers to one or more of the compounds described herein, such as, e.g., an EV mixed or intermingled with, or suspended in one or more other chemical components, such as pharmaceutically-acceptable carriers and excipients. One purpose of a pharmaceutical composition is to facilitate administration of preparations of EVs to a subject. The term “excipient” or “carrier” refers to an inert substance added to a pharmaceutical composition to further facilitate administration of a compound. The term “pharmaceutically-acceptable carrier” or “pharmaceutically-acceptable excipient” and grammatical variations thereof, encompasses any of the agents approved by a regulatory agency of the US Federal government or listed in the US Pharmacopeia for use in animals, including humans, as well as any carrier or diluent that does not cause the production of undesirable physiological effects to a degree that prohibits administration of the composition to a subject and does not abrogate the biological activity and properties of the administered compound. Included are excipients and carriers that are useful in preparing a pharmaceutical composition and are generally safe, non-toxic, and desirable.

As used herein, the term “payload” refers to a therapeutic agent that acts on a target (e.g., a target cell) that is contacted with the EV. Payloads that can be introduced into an EV and/or a producer cell include therapeutic agents such as, nucleotides (e.g., nucleotides comprising a detectable moiety or a toxin or that disrupt transcription), nucleic acids (e.g., DNA or mRNA molecules that encode a polypeptide such as an enzyme, or RNA molecules that have regulatory function such as miRNA, dsDNA, lncRNA, and siRNA), amino acids (e.g., amino acids comprising a detectable moiety or a toxin or that disrupt translation), polypeptides (e.g., enzymes), lipids, carbohydrates, and small molecules (e.g., small molecule drugs and toxins).

The terms “administration,” “administering” and variants thereof refer to introducing a composition, such as an EV, or agent into a subject and includes concurrent and sequential introduction of a composition or agent. The introduction of a composition or agent into a subject is by any suitable route, including intratumorally, orally, pulmonarily, intranasally, parenterally (intravenously, intra-arterially, intramuscularly, intraperitoneally, or subcutaneously), rectally, intralymphatically, intrathecally, periocularly or topically. Administration includes self-administration and the administration by another. A suitable route of administration allows the composition or the agent to perform its intended function. For example, if a suitable route is intravenous, the composition is administered by introducing the composition or agent into a vein of the subject.

The terms “individual,” “subject,” “host,” and “patient,” are used interchangeably herein and refer to any mammalian subject for whom diagnosis, treatment, or therapy is desired, particularly humans. The compositions and methods described herein are applicable to both human therapy and veterinary applications. In some aspects, the subject is a mammal, and in other aspects the subject is a human. As used herein, a “mammalian subject” includes all mammals, including without limitation, humans, domestic animals (e.g., dogs, cats and the like), farm animals (e.g., cows, sheep, pigs, horses and the like) and laboratory animals (e.g., monkey, rats, mice, rabbits, guinea pigs and the like).

As used herein, the term “substantially free” means that the sample comprising EVs, e.g., exosomes, comprise less than 10% of macromolecules by mass/volume (m/v) percentage concentration. Some fractions may contain less than 0.001%, less than 0.01%, less than 0.05%, less than 0.1%, less than 0.2%, less than 0.3%, less than 0.4%, less than 0.5%, less than 0.6%, less than 0.7%, less than 0.8%, less than 0.9%, less than 1%, less than 2%, less than 3%, less than 4%, less than 5%, less than 6%, less than 7%, less than 8%, less than 9%, or less than 10% (m/v) of macromolecules.

As used herein, “perfusion culture” refers to cell culturing methods or cell cultures in which cells are continuously fed with fresh media and spent media is continuously removed while keeping the cells in the culture vessel. In some aspects, culture vessels for perfusion culture comprise cell retention devices, such as capillary fibers or membranes. Examples of perfusion bioreactors with cell retention devices include bioreactors, such as the N-terminal production vessel, such as a stirred-tank bioreactor, connected to a cell retention device, such as hollow fiber filters or an acoustic cell separator. In some aspects, the perfusion culture is single-cell perfusion culture. In some aspects, the perfusion culture comprises use of a single-cell suspension perfusion bioreactor wherein individual cells are isolated for addition of fresh medium and removal of spent medium. In some aspects, the perfusion culture comprises separating cells from spent medium by centrifugation.

As used herein, “fed-batch” refers to cell culturing methods or cell cultures wherein cells remain in the culturing vessel until harvesting of the cells, in which an initial culture medium is added to an initial cell culture and additional feed medium is added to prevent nutrient depletion. In some aspects, feed medium is added once during the culturing process. In some aspects, the feed medium is added multiple times during the culturing process. In some aspects, fed-batch culture can include constantly-fed batch culture and exponential-fed batch culture.

“Treat,” “treatment,” or “treating,” as used herein refers to, e.g., the reduction in severity of a disease or condition; the reduction in the duration of a disease course; the amelioration or elimination of one or more symptoms associated with a disease or condition; the provision of beneficial effects to a subject with a disease or condition, without necessarily curing the disease or condition. The term also includes prophylaxis or prevention of a disease or condition or its symptoms thereof. In one aspect, the term “treating” or “treatment” means inducing an immune response in a subject against an antigen. In some aspects, the disease or condition is a cancer.

“Prevent” or “preventing,” as used herein, refers to decreasing or reducing the occurrence or severity of a particular outcome. In some aspects, preventing an outcome is achieved through prophylactic treatment. In some aspects, an EV, e.g., an exosome, comprising a cytokine, e.g., an IL-12 moeity, described herein is administered to a subject prophylactically.

As used herein, the term “immune modulator” refers to an agent (i.e., therapeutic agent) that acts on a target (e.g., a target cell) that is contacted with the extracellular vesicle, and regulates the immune system. Non-limiting examples of immune modulator that can be introduced into an EV (e.g., exosome) and/or a producer cell include agents such as, modulators of checkpoint inhibitors, ligands of checkpoint inhibitors, cytokines, derivatives thereof, or any combination thereof. The immune modulator can also include an agonist, an antagonist, an antibody, an antigen-binding fragment, a polynucleotide, such as siRNA, antisense oligonucleotide, a phosphorodiamidate morpholino oligomer (PMO), a peptide-conjugated phosphorodiamidate morpholino oligomer (PPMO), miRNA, lncRNA, mRNA DNA, or a small molecule.

### II. Method of Producing Extracellular Vesicles

The present disclosure provides methods of increasing a number of extracellular vesicles (EVs) produced from producer cells, comprising modifying the producer cells to exhibit a reduced gene and/or protein function in a cholesterol biosynthetic pathway of the producer cells. The present disclosure also provides methods of producing extracellular vesicles (EVs) from a producer cells, comprising culturing the producer cells, which exhibit a reduced gene and/or protein function in a cholesterol biosynthetic pathway.

Without wishing to be bound by theory, Applicant has surprisingly discovered that genetic and/or pharmacological inhibition of a cholesterol synthesis pathway in producer cells increased the yield of harvested EVs compared to producer cells that did not have a genetic and/or pharmacological inhibition of a cholesterol synthesis pathway. These results are in stark contrast to previous studies that found that cholesterol levels had a positive effect on exosome secretion. In particular, a study conducted by Kulshreshtha et al. (Scientific Reports, 2019, 9:16373) found that producer cells treated with simvastatin reduced exosome secretion. Similarly, a study by Baek et al. (Endocrinology 162(7):1-18 (2021)) found that culture of primary murine polymorphonuclear neutrophils, RAW264.7 monocytic cells, and 4T1 murine mammary cancer cells in the presence the cholesterol metabolite 27-hydroxycholesterol (27HC) led to increased exosome production, suggesting that increased cholesterol levels lead to increased exosome production. Finally, an analysis by Guix et al. reported in Life Science Alliance (vol. 4, no. 8, e202101055, June, 2021) showed that accumulation of cholesterol in the multivesicular bodies of aging neurons in culture correlates with increased secretion of small extracellular vesicles as compared to younger neurons, again suggesting that increased cholesterol is associated with increased extracellular vesicle production. As such, the results presented herein showing that cholesterol inhibition in various producer cells using different statins consistently resulted in increased exosome production would not have been expected in view of the state of the field at the time of the present application.

In some aspects, expression of one or more genes in a cholesterol synthesis pathway or their proteins can be inhibited or reduced. Non-limiting examples of the genes include HMG CoA (3-hydroxy-3-methylglutaryl coenzyme A), SQLE (squalene monooxygenase), ACAT1 (acetyl-CoA acetyltransferase 1), ACAT2 (acetyl-CoA acetyltransferase 2), HMGCS1 (3-hydroxy-3-methylglutaryl-CoA synthase 1), HMGCS2 (3-hydroxy-3-methylglutaryl-CoA synthase 2), HMGCR (3-hydroxy-3-methylglutaryl-CoA reductase), MVK (mevalonate kinase), PMVK (phosphomevalonate kinase), MVD (mevalonate decarboxylase), IDI1 (isopentenyl-diphosphate delta isomerase 1), IDI2 (isopentenyl-diphosphate delta isomerase 2), FDPS (farnesyl diphosphate synthase), GGPS1 (geranylgeranyl diphosphate synthase 1), FDFT1 (farnesyl-diphosphate farnesyltransferase 1), SQLE (squalene epoxidase), LSS (lanosterol synthase), DHCR24 (24-dehydrocholesterol reductase), CYP51A1 (cytochrome P450 family 51 subfamily A polypeptide 1), TM7SF2 (transmembrane 7 superfamily member 2), FAXDC2 (C5orf4fatty acid hydroxylase domain containing 2), MSMO1 (SC4MOL methylsterol monooxygenase), NSDHL (NAD(P) dependent steroid dehydrogenase-like), HSD17B7 (hydroxysteroid (17-beta) dehydrogenase 7), EBP (emopamil binding protein (sterol isomerase), SC5D (SC5DL sterol-C5-desaturase Synthesis), DHCR7 (7-dehydrocholesterol reductase), CEL (carboxyl ester lipase), LIPA (lipase A, lysosomal acid), SOAT1 (sterol O-acetyltransferase 1), SOAT2 (sterol O-acetyltransferase 2), ABCA1 (ATP-binding cassette, sub-family A), ABCG1 (ATP-binding cassette, sub-family G), SLCO2B1 (solute carrier organic anion transporter family member 2B1), SLCO1B3 (solute carrier organic anion transporter family member 1B3), LDLR (low density lipoprotein receptor), APOE (apolipoprotein E), SREBF1 (sterol binding element transcription factor 1), SREBF2 (sterol binding element transcription factor 2), SCAP (SREBF chaperone), MBTPS1 (SIP membrane bound transcription factor peptidase site 1), MBTPS2 (S2P, membrane bound transcription factor peptidase site 2), INSIG1 (insulin induced gene 1), INSIG2 (insulin induced gene 2), AMFR (GP78, autocrine motility factor receptor E3 ubiquitin protein ligase), NR1H3 (LXRA, nuclear receptor subfamily 1 group H member 3), NR1H2 (LXRB, nuclear receptor subfamily 1 group H member 2), RXRA (retinoid X receptor alpha), RXRB (retinoid X receptor beta), or MYLIP (IDOL, myosin regulatory light chain interacting protein). In some aspects, the gene is HMGCR. In other aspects, the gene is SREBF2.

In some aspects, the expression of SM (squalene monooxygenase) or its protein can be inhibited or reduced. In some aspects, the expression of ACAT1 (acetyl-CoA acetyltransferase 1) or its protein can be inhibited or reduced. In some aspects, the expression of ACAT2 (acetyl-CoA acetyltransferase 2) or its protein can be inhibited or reduced. In some aspects, the expression of HMGCS2 (3-hydroxy-3-methylglutaryl-CoA synthase 2) or its protein can be inhibited or reduced. In some aspects, the expression of MVK (mevalonate kinase) or its protein can be inhibited or reduced. In some aspects, the expression of PMVK (phosphomevalonate kinase) or its protein can be inhibited or reduced. In some aspects, the expression of IDI1 (isopentenyl-diphosphate delta isomerase 1) or its protein can be inhibited or reduced. In some aspects, the expression of IDI2 (isopentenyl-diphosphate delta isomerase 2) or its protein can be inhibited or reduced. In some aspects, the expression of FDPS (farnesyl diphosphate synthase) or its protein can be inhibited or reduced. In some aspects, the expression of GGPS1 (geranylgeranyl diphosphate synthase 1) or its protein can be inhibited or reduced. In some aspects, the expression of FDFT1 (famesyl-diphosphate farnesyltransferase 1) or its protein can be inhibited or reduced. In some aspects, the expression of SQLE (squalene epoxidase) or its protein can be inhibited or reduced. In some aspects, the expression of LSS (lanosterol synthase) or its protein can be inhibited or reduced. In some aspects, the expression of DHCR24 (24-dehydrocholesterol reductase) or its protein can be inhibited or reduced. In some aspects, the expression of CYP51A1 (cytochrome P450 family 51 subfamily A polypeptide 1) or its protein can be inhibited or reduced. In some aspects, the expression of TM7SF2 (transmembrane 7 superfamily member 2) or its protein can be inhibited or reduced. In some aspects, the expression of FAXDC2 (C5orf4 fatty acid hydroxylase domain containing 2) or its protein can be inhibited or reduced. In some aspects, the expression of MSMO1 (SC4MOL methylsterol monooxygenase) or its protein can be inhibited or reduced. In some aspects, the expression of NSDHL (NAD(P) dependent steroid dehydrogenase-like) or its protein can be inhibited or reduced. In some aspects, the expression of EBP (emopamil binding protein (sterol isomerase) or its protein can be inhibited or reduced. In some aspects, the expression of HSD17B7 (hydroxysteroid (17-beta) dehydrogenase 7) or its protein can be inhibited or reduced. In some aspects, the expression of DHCR7 (7-dehydrocholesterol reductase) or its protein can be inhibited or reduced. In some aspects, the expression of CEL (carboxyl ester lipase) or its protein can be inhibited or reduced. In some aspects, the expression of LIPA (lipase A, lysosomal acid) or its protein can be inhibited or reduced. In some aspects, the expression of SOAT1 (sterol 0-acetyltransferase 1) or its protein can be inhibited or reduced. In some aspects, the expression of SOAT2 (sterol O-acetyltransferase 2) or its protein can be inhibited or reduced. In some aspects, the expression of ABCA1 (ATP-binding cassette, sub-family A) or its protein can be inhibited or reduced. In some aspects, the expression of SLCO2B1 (solute carrier organic anion transporter family member 2B1) or its protein can be inhibited or reduced. In some aspects, the expression of SLCO1B3 (solute carrier organic anion transporter family member 1B3) or its protein can be inhibited or reduced. In some aspects, the expression of APOE (apolipoprotein E) or its protein can be inhibited or reduced. In some aspects, the expression of SREBF1 (sterol binding element transcription factor 1) or its protein can be inhibited or reduced. In some aspects, the expression of SREBF2 (sterol binding element transcription factor 2) or its protein can be inhibited or reduced. In some aspects, the expression of SCAP (SREBF chaperone) can be inhibited or reduced. In some aspects, the expression of MBTPS1 (SiP membrane bound transcription factor peptidase site 1) or its protein can be inhibited or reduced. In some aspects, the expression of MBTPS2 (S2P, membrane bound transcription factor peptidase site 2) or its protein can be inhibited or reduced. In some aspects, the expression of INSIG1 (insulin induced gene 1) or its protein can be inhibited or reduced. In some aspects, the expression of INSIG2 (insulin induced gene 2) or its protein can be inhibited or reduced. In some aspects, the expression of AMFR (GP78, autocrine motility factor receptor E3 ubiquitin protein ligase) or its protein can be inhibited or reduced. In some aspects, the expression of NR1H3 (LXRA, nuclear receptor subfamily 1 group H member 3) or its protein can be inhibited or reduced. In some aspects, the expression of NR1H2 (LXRB, nuclear receptor subfamily 1 group H member 2) or its protein can be inhibited or reduced. In some aspects, the expression of RXRA (retinoid X receptor alpha) or its protein can be inhibited or reduced. In some aspects, the expression of RXRB (retinoid X receptor beta) or its protein can be inhibited or reduced. In some aspects, the expression of MYLIP (IDOL, myosin regulatory light chain interacting protein) or its protein can be inhibited or reduced.

### HMGCR

3-hydroxy-3-methylglutaryl-coenzyme A reductase, generally abbreviated HMG Co-A reductase or HMGCR, and also known as LDLCQ3, is a protein which in humans is encoded by the HMGCR gene. The HMGCR gene is located on chromosome 5 (bases 75,336,334 to 75,362,116; NCBI Reference Sequence: NC_000005.10). HMGCR catalyzes the conversion of HMG-CoA to mevalonate and is the rate-limiting enzyme for cholesterol synthesis. A negative feedback mechanism mediated by sterols and non-sterol metabolites derived from mevalonate regulates HMGCR enzymatic activity. Normally in mammalian cells HMGCR is suppressed by cholesterol derived from the internalization and degradation of low density lipoprotein (LDL) via the LDL receptor.

The HMGCR proteins have 3 isoforms produced by alternative splicing. The sequences are shown in Table 1 below.

TABLE 1
HMGCR protein isoforms.

| | |
|---|---|
| HMGCR | MLSRLFRMHGLFVASHPWEVIVGTVTLTICMM |
| Isoform 1 | SMNMFTGNNKICGWNYECPKFEEDVLSSDIII |
| (identifier: | LTITRCIAILYIYFQFQNLRQLGSKYILGIAG |
| P04035-1) | LFTIFSSFVFSTVVIHFLDKELTGLNEALPFF |
| (SEQ ID NO: | LLLIDLSRASTLAKFALSSNSQDEVRENIARG |
| XX) (also | MAILGPTFTLDALVECLVIGVGTMSGVRQLEI |
| known as | MCCFGCMSVLANYFVFMTFFPACVSLVLELSR |
| HMGCR-1a) | ESREGRPIWQLSHFARVLEEEENKPNPVTQRV |
| | KMIMSLGLVLVHAHSRWIADPSPQNSTADTSK |
| | VSLGLDENVSKRIEPSVSLWQFYLSKMISMDI |
| | EQVITLSLALLLAVKYIFFEQTETESTLSLKN |
| | PITSPVVTQKKVPDNCCRREPMLVRNNQKCDS |
| | VEEETGINRERKVEVIKPLVAETDTPNRATFV |
| | VGNSSLLDTSSVLVTQEPEIELPREPRPNEEC |
| | LQILGNAEKGAKFLSDAEIIQLVNAKHIPAYK |
| | LETLMETHERGVSIRRQLLSKKLSEPSSLQYL |
| | PYRDYNYSLVMGACCENVIGYMPIPVGVAGPL |
| | CLDEKEFQVPMATTEGCLVASTNRGCRAIGLG |
| | GGASSRVLADGMTRGPVVRLPRACDSAEVKAW |
| | LETSEGFAVIKEAFDSTSRFARLQKLHTSIAG |
| | RNLYIRFQSRSGDAMGMNMISKGTEKALSKLH |
| | EYFPEMQILAVSGNYCTDKKPAAINWIEGRGK |
| | SVVCEAVIPAKVVREVLKTTTEAMIEVNINKN |
| | LVGSAMAGSIGGYNAHAANIVTAIYIACGQDA |
| | AQNVGSSNCITLMEASGPTNEDLYISCTMPSI |
| | EIGTVGGGTNLLPQQACLQMLGVQGACKDNPG |
| | ENARQLARIVCGTVMAGELSLMAALAAGHLVK |
| | SHMIHNRSKINLQDLQGACTKKTA |
| HMGCR | MLSRLFRMHGLFVASHPWEVIVGTVTLTICMM |
| Isoform 2 | SMNMFTGNNKICGWNYECPKFEEDVLSSDIII |
| (identifier: | LTITRCIAILYIYFQFQNLRQLGSKYILGIAG |
| P04035-2) | LFTIFSSFVFSTVVIHFLDKELTGLNEALPFF |
| (SEQ ID NO: | LLLIDLSRASTLAKFALSSNSQDEVRENIARG |
| XX) (also | MAILGPTFTLDALVECLVIGVGTMSGVRQLEI |
| known as | MCCFGCMSVLANYFVFMTFFPACVSLVLELSR |
| HMGCR-1c | ESREGRPIWQLSHFARVLEEEENKPNPVTQRV |
| | KMIMSLGLVLVHAHSRWIADPSPQNSTADTSK |
| | VSLGLDENVSKRIEPSVSLWQFYLSKMISMDI |
| | EQVITLSLALLLAVKYIFFEQTETESTLSLKN |
| | PITSPVVTQKKVPDNCCRREPMLVRNNQKCDS |
| | VEEETGINRERKVEVIKPLVAETDTPNRATFV |
| | VGNSSLLDTSSVLVTQEPEIELPREPRPNEEC |
| | LQILGNAEKGAKFLSDAEIIQLVNAKHIPAYK |
| | LETLMETHERGVSIRRQLLSKKLSEPSSLQYL |
| | PYRDYNYSLLGGGASSRVLADGMTRGPVVRLP |
| | RACDSAEVKAWLETSEGFAVIKEAFDSTSRFA |
| | RLQKLHTSIAGRNLYIRFQSRSGDAMGMNMIS |
| | KGTEKALSKLHEYFPEMQILAVSGNYCTDKKP |
| | AAINWIEGRGKSVVCEAVIPAKVVREVLKTTT |
| | EAMIEVNINKNLVGSAMAGSIGGYNAHAANIV |
| | TAIYIACGQDAAQNVGSSNCITLMEASGPTNE |
| | DLYISCTMPSIEIGTVGGGTNLLPQQACLQML |
| | GVQGACKDNPGENARQLARIVCGTVMAGELSL |
| | MAALAAGHLVKSHMIHNRSKINLQDLQGACTK |
| | KTA |
| HMGCR | MQWMSHTRERDAGSKDSVATMLSRLFRMHGLF |
| Isoform 3 | VASHPWEVIVGTVTLTICMMSMNMFTGNNKIC |
| (identifier: | GWNYECPKFEEDVLSSDIIILTITRCIAILYI |
| Q92570-3) | YFQFQNLRQLGSKYILGIAGLFTIFSSFVFST |
| (also known | VVIHFLDKELTGLNEALPFFLLLIDLSRASTL |
| as HMGCR-1b) | AKFALSSNSQDEVRENIARGMAILGPTFTLDA |
| | LVECLVIGVGTMSGVRQLEIMCCFGCMSVLAN |
| | YFVFMTFFPACVSLVLELSRESREGRPIWQLS |
| | HFARVLEEEENKPNPVTQRVKMIMSLGLVLVH |
| | AHSRWIADPSPQNSTADTSKVSLGLDENVSKR |
| | IEPSVSLWQFYLSKMISMDIEQVITLSLALLL |
| | AVKYIFFEQTETESTLSLKNPITSPVVTQKKV |
| | PDNCCRREPMLVRNNQKCDSVEEETGINRERK |
| | VEVIKPLVAETDTPNRATFVVGNSSLLDTSSV |
| | LVTQEPEIELPREPRPNEECLQILGNAEKGAK |
| | FLSDAEIIQLVNAKHIPAYKLETLMETHERGV |
| | SIRRQLLSKKLSEPSSLQYLPYRDYNYSLVMG |
| | ACCENVIGYMPIPVGVAGPLCLDEKEFQVPMA |
| | TTEGCLVASTNRGCRAIGLGGGASSRVLADGM |
| | TRGPVVRLPRACDSAEVKAWLETSEGFAVIKE |
| | AFDSTSRFARLQKLHTSIAGRNLYIRFQSRSG |
| | DAMGMNMISKGTEKALSKLHEYFPEMQILAVS |
| | GNYCTDKKPAAINWIEGRGKSVVCEAVIPAKV |
| | VREVLKTTTEAMIEVNINKNLVGSAMAGSIGG |
| | YNAHAANIVTAIYIACGQDAAQNVGSSNCITL |
| | MEASGPTNEDLYISCTMPSIEIGTVGGGTNLL |
| | PQQACLQMLGVQGACKDNPGENARQLARIVCG |
| | TVMAGELSLMAALAAGHLVKSHMIHNRSKINL |
| | QDLQGACTKKTA |

In some aspects, the methods of the present disclosure comprise modifying the producer cells to exhibit a reduced gene and/or protein function in a cholesterol biosynthetic pathway of the producer cells. In some aspects, the methods of the present disclosure comprise culturing the producer cells, which exhibit a reduced gene and/or protein function in a cholesterol biosynthetic pathway. In some aspects, the reduced gene and/or protein in a cholesterol biosynthesis pathway comprises HMGCR gene and/or HMGCR protein. As used herein, the term “HMGCR gene” refers to any transcript, genomic DNA, pre-mRNA, or mRNA. As used herein, “HMGCR protein” refers to HMGCR isoform 1, HMGCR isoform 2, or HMGCR isoform 3, disclosed above, as well as variants and mutants thereof. As used herein, the term HMGCR protein also encompasses any fragment or variant of any of the isoforms disclosed herein that has at least one function of the wild type HMGCR protein.

As used herein the term “reduced gene and/or protein function” refers both to reduction in physical levels (e.g., less gene sequence due to edition from the genome, or less protein due a decrease in protein expression) and to reduction in function. For example, a reduction in level of HMGCR gene can refer to a decrease in gene function, e.g., due to the introduction of a mutation introducing a stop codon or a frame shift, to an epigenetic modification that would alter transcription, or to a mutation or other change on a promoter gene or another gene that regulates HMGCR expression. In some aspects, a reduction in level of HMGCR gene in a modified cell refers to a decrease in the amount (e.g., concentration) of genomic DNA, pre-mRNA, and/or mRNA that is capable of encoding a functional HMGCR protein, e.g., wild type HMGCR protein, compared to a reference (e.g., untreated) cell. Similarly, a reduction in HMGCR protein can refer to changes resulting in the expression of a functional HMGCR protein, e.g., wild type HMGCR protein, including but not limited to changes (e.g., mutations or post-translational modifications) that cause a loss of function (partial or complete), or to the activity of molecules that bind to functional sites of HMGCR altering, e.g., its enzyme activity.

HMGCR gene levels (e.g., presence/absence of the entire gene or a portion thereof, or gene function) can be measured by various methods known in the art. HMGCR protein levels (e.g., presence/absence of the HMGCR protein or fragments thereof, or quantification or protein function) can be measured by various methods known in the art.

In some aspects, the reduced expression levels of HMGCR gene and/or expression or functional levels of HMGCR protein in the producer cells can increase the yield of the EVs produced by the producer cells compared to EVs produced by a producer cells that do not have a reduced expression levels of HMGCR gene and/or expression levels of HMGCR protein. In some aspects, the yield is increased at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 16 fold, at least about 17 fold, at least about 18 fold, at least about 19 fold, at least about 20 fold, at least about 21 fold, at least about 22 fold, at least about 23 fold, at least about 24 fold, at least about 25 fold, at least about 26 fold, at least about 27 fold, at least about 28 fold, at least about 29 fold, or at least about 30 fold. In some aspects, the yield is increased about 2 fold to about 30 fold, about 2 fold to about 25 fold, about 2 fold to about 20 fold, about 2 fold to about 15 fold, about 12 fold to about 10 fold, about 2 fold to about 5 fold, about 5 fold to about 30 fold, about 5 fold to about 25 fold, about 5 fold to about 20 fold, about 5 fold to about 15 fold, about 5 fold to about 10 fold, about 10 fold to about 30 fold, about 10 fold to about 25 fold, about 10 fold to about 20 fold, about 10 fold to about 15 fold, about 15 fold to about 30 fold, about 15 fold to about 25 fold, about 15 fold to about 20 fold, about 20 fold to about 30 fold, about 20 fold to about 25 fold, or about 25 fold to about 30 fold. In some aspects, the yield is increased about 2 fold, about 3 fold, about 4 fold, about 5 fold, about 6 fold, about 7 fold, about 8 fold, about 9 fold, or about 10 fold.

In some aspects, the HMGCR gene expression is reduced about 2 to about 20 fold. In some aspects, the HMGCR gene expression is reduced about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, or about 20 fold.

In some aspects, the gene and/protein function in a cholesterol biosynthetic pathway is reduced at least about 90%, at least about 80%, at least about 70%, at least about 60%, at least about 50%, at least about 40%, at least about 30%, at least about 20%, or at least about 10%, at least about 5%, or at least about 1%. In some aspects, the gene and/protein function in a cholesterol biosynthetic pathway is reduced at least about 90%, at least about 80%, at least about 70%, at least about 60%, at least about 50%, at least about 40%, or at least about 30%.

In some aspects, the modifying of the methods described herein comprises contacting the producer cells with an agent capable of reducing the HMGCR gene and/or HMGCR protein function. In some aspects, the agent comprises a statin. Without wishing to be bound by theory, statins work by competitively blocking the active site of the first and key rate-limiting enzyme in the mevalonate pathway (e.g., cholesterol biosynthetic pathway), HMG-CoA reductase (HMGCR). Inhibition of this site prevents substrate access, thereby blocking the conversion of HMG-CoA to mevalonic acid. The active component of statins is a modified 3,5-dihydroxyglutaric acid moiety, which is structurally similar to the endogenous substrate, HMG-CoA, and the mevaldyl CoA transition state intermediate. This active site binds to and inhibits HMG-CoA reductase activity in a stereoselective process that requires the statin to have a 3R,5R configuration. The molecular and clinical differences of statins arise from the ring that is attached to the active moiety, which can be a partially reduced naphthalene (lovastatin, simvastatin, pravastatin), a pyrrole (atorvastatin), an indole (fluvastatin), a pyrimidine (rosuvastatin), a pyridine (cerivastatin), or a quinoline (pitavastatin). The substituents on the ring define the solubility and pharmacological properties of the statin. Hydrophilicity (pravastatin and rosuvastatin) originates from the common active site plus other polar substituents, whereas lipophilicity (atorvastatin, lovastatin, fluvastatin, pitavastatin, simvastatin, and cerivastatin) arises because of the addition of nonpolar substituents. In some aspects, the statin can be selected from atorvastatin, lovastatin, pitavastatin, pravastatin, fluvastatin, cerivastatin, rosuvastatin, mevastatin, simvastatin, or combinations thereof. In some aspects, the statin comprises simvastatin. In some aspects, the statin comprises lovastatin, simvastatin, and/or pravastatin. In some aspects, the statin comprises atorvastatin. In some aspects, the statin comprises fluvastatin. In some aspects, the statin comprises rosuvastatin. In some aspects, the statin comprises cerivastatin. In some aspects, the statin comprises pitavastatin. In some aspects, the stating comprises pravastatin and/or rosuvastatin. In some aspects, the statin comprises atorvastatin, lovastatin, fluvastatin, pitavastatin, simvastatin, and/or cerivastatin.

In some aspects, the statin (e.g., simvastatin) is contacted with the producer cells at a concentration of about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, or about 20 nM, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 260 nM, about 270 nM, about 280 nM, about 290 nM, about 300 nM, about 310 nM, about 320 nM, about 330 nM, about 340 nM, about 350 nM, about 360 nM, about 370 nM, about 380 nM, about 390 nM, about 400 nM, about 410 nM, about 420 nM, about 430 nM, about 440 nM, about 450 nM, about 460 nM, about 470 nM, about 480 nM, about 490 nM, about 500 nM, about 510 nM, about 520 nM, about 530 nM, about 540 nM, about 550 nM, about 560 nM, about 570 nM, about 580 nM, about 590 nM, about 600 nM, about 610 nM, about 620 nM, about 630 nM, about 640 nM, about 650 nM, about 660 nM, about 670 nM, about 680 nM, about 690 nM, about 700 nM, about 710 nM, about 720 nM, about 730 nM, about 740 nM, about 750 nM, about 760 nM, about 770 nM, about 780 nM, about 790 nM, about 800 nM, about 810 nM, about 820 nM, about 830 nM, about 840 nM, about 850 nM, about 860 nM, about 870 nM, about 880 nM, about 890 nM, about 900 nM, about 910 nM, about 920 nM, about 930 nM, about 940 nM, about 950 nM, about 960 nM, about 970 nM, about 980 nM, about 990 nM, or about 1,000 nM. In some aspects, the statin is contacted at a concentration of between about 0.1 nM to about 100 nM, about 0.1 nM to about 90 nM, about 0.1 nM to about 80 nM, about 0.1 nM to about 70 nM, about 0.1 nM to about 60 nM, about 0.1 nM to about 50 nM, about 0.1 nM to about 40 nM, about 0.1 nM to about 30 nM, about 0.1 nM to about 20 nM, 0.1 nM to about 10 nM, or about 1 nM to about 20 nM, about 1 nM to about 10 nM, about 1 nM to about 5 nM, about 5 nM to about 20 nM, about 5 nM to about 15 nM, about 5 nM to about 10 nM, about 10 nM to about 50 nM, about 10 nM to about 40 nM, about 10 nM to about 30 nM, about 10 nM to about 20 nM, about 1 nM to about 10 nM, or about 10 nM to about 20 nM.

In some aspects, the statin (e.g., simvastatin) is contacted with the producer cells at a concentration of about 1 nM to about 10 nM.

In some aspects, the statin is contacted with the producer cells prior to harvesting EVs. In some aspects, the statin is contacted with the producer cells about 1 hour, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, or about 24 hours prior to harvesting the EVs. In some aspects, the statin is contacted with the producer cells about 24 hours, about 36 hours, about 48 hours, about 60 hours, about 72 hours, about 84 hours, about 96 hours, about 108 hours, about 120 hours, about 132 hours, about 144 hours, about 156 hours, about 168 hours, about 180 hours, about 192 hours, about 204 hours, about 216 hours, about 228 hours, or about 240 hours. In some aspects, the statin is contacted with the producer cells between about 10 days to about 15 days, between about 10 days to about 20 days, between about 10 days to about 25 days, between about 10 days to about 30 days, between about 10 days to about 35 days, or between about 10 days to about 40 days prior to harvesting the EVs. In some aspects, the statin is contacted with the producer cells while EVs are harvested. In some aspects, the statin is contacted with the producer cells over the duration of a continuous (e.g., perfusion) cell culture process that spans about 30 days, about 35 days, or about 40 days.

In some aspect, the agent comprises Meglutol, Clinofibrate, Hesperetin 7-O-glucoside, Cmpd 81 (also named HMG499, and related structures, described in Jiang et al., Nature Communications 9, 5138 (2018), which is herein incorporated by reference in its entirety), a PROTAC, or Monacolin J.

In some aspects, the producer cells are modified prior to the culturing by contacting the producer cells with an agent capable of reducing the gene and/or protein function in a cholesterol biosynthetic pathway.

In some aspects, the agent is contacted with the producer cells at a concentration of about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, or about 20 nM, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 260 nM, about 270 nM, about 280 nM, about 290 nM, about 300 nM, about 310 nM, about 320 nM, about 330 nM, about 340 nM, about 350 nM, about 360 nM, about 370 nM, about 380 nM, about 390 nM, about 400 nM, about 410 nM, about 420 nM, about 430 nM, about 440 nM, about 450 nM, about 460 nM, about 470 nM, about 480 nM, about 490 nM, about 500 nM, about 510 nM, about 520 nM, about 530 nM, about 540 nM, about 550 nM, about 560 nM, about 570 nM, about 580 nM, about 590 nM, about 600 nM, about 610 nM, about 620 nM, about 630 nM, about 640 nM, about 650 nM, about 660 nM, about 670 nM, about 680 nM, about 690 nM, about 700 nM, about 710 nM, about 720 nM, about 730 nM, about 740 nM, about 750 nM, about 760 nM, about 770 nM, about 780 nM, about 790 nM, about 800 nM, about 810 nM, about 820 nM, about 830 nM, about 840 nM, about 850 nM, about 860 nM, about 870 nM, about 880 nM, about 890 nM, about 900 nM, about 910 nM, about 920 nM, about 930 nM, about 940 nM, about 950 nM, about 960 nM, about 970 nM, about 980 nM, about 990 nM, or about 1,000 nM. In some aspects, the statin is contacted at a concentration of between about 0.1 nM to about 100 nM, about 0.1 nM to about 90 nM, about 0.1 nM to about 80 nM, about 0.1 nM to about 70 nM, about 0.1 nM to about 60 nM, about 0.1 nM to about 50 nM, about 0.1 nM to about 40 nM, about 0.1 nM to about 30 nM, about 0.1 nM to about 20 nM, 0.1 nM to about 10 nM, or about 1 nM to about 20 nM, about 1 nM to about 10 nM, about 1 nM to about 5 nM, about 5 nM to about 20 nM, about 5 nM to about 15 nM, about 5 nM to about 10 nM, about 10 nM to about 50 nM, about 10 nM to about 40 nM, about 10 nM to about 30 nM, about 10 nM to about 20 nM, about 1 nM to about 10 nM, or about 10 nM to about 20 nM.

In some aspects, the agent capable of reducing the gene and/or protein function in a cholesterol biosynthetic pathway comprises a gene editing technology. In some aspects, the gene editing technology comprises a shRNA, siRNA, miRNA, antisense oligonucleotides, CRISPR, zinc finger nuclease, TALEN, meganuclease, restriction endonuclease, or any combination thereof. In some aspects, the gene editing technology comprises siRNA.

### SREBF2

Sterol regulatory element binding factor 2, generally abbreviated SREBF2, and also known as lo, nu, SRE, nuc, SREB, SREBP, bHLHd, lop13, SREBP2, bHLHd2, SREBP-2, AI608257, and SREBP2gc, is a protein which in human is encoded by the SREBF2 gene. The SREBF2 gene is located on chromosome 15 (bases 82,031,470 to 82,089,580; NCBI Reference Sequence: NC_000081.7). SREBF2 is a transcription factor that is the master regulator of cholesterol synthesis, activating expression of genes such as HMG-CoA reductase (HMGCR), HMG-CoA synthase (HMGCS), and mevalonate kinase (MVK).

The SREBF2 protein has two isoforms produced by alternative splicing. The sequences are shown in Table 2 below.

TABLE 2
HMGCR protein isoforms.

| | |
|---|---|
| SREBF2 | MDDSGELGGLETMETLTELGDELTLGDIDEML |
| Isoform 1 | QFVSNQVGEFPDLFSEQLCSSFPGSGGSGSSS |
| (identifier: | GSSGSSSSSSNGRGSSSGAVDPSVQRSFTQVT |
| Q12772-1) | LPSFSPSAASPQAPTLQVKVSPTSVPTTPRAT |
| (SEQ ID NO: | PILQPRPQPQPQPQTQLQQQTVMITPTFSTTP |
| XX) | QTRIIQQPLIYQNAATSFQVLQPQVQSLVTSS |
| | QVQPVTIQQQVQTVQAQRVLTQTANGTLQTLA |
| | PATVQTVAAPQVQQVPVLVQPQIIKTDSLVLT |
| | TLKTDGSPVMAAVQNPALTALTTPIQTAALQV |
| | PTLVGSSGTILTTMPVMMGQEKVPIKQVPGGV |
| | KQLEPPKEGERRTTHNIIEKRYRSSINDKIIE |
| | LKDLVMGTDAKMHKSGVLRKAIDYIKYLQQVN |
| | HKLRQENMVLKLANQKNKLLKGIDLGSLVDNE |
| | VDLKIEDFNQNVLLMSPPASDSGSQAGFSPYS |
| | IDSEPGSPLLDDAKVKDEPDSPPVALGMVDRS |
| | RILLCVLTFLCLSFNPLTSLLQWGGAHDSDQH |
| | PHSGSGRSVLSFESGSGGWFDWMMPTLLLWLV |
| | NGVIVLSVFVKLLVHGEPVIRPHSRSSVTFWR |
| | HRKQADLDLARGDFAAAAGNLQTCLAVLGRAL |
| | PTSRLDLACSLSWNVIRYSLQKLRLVRWLLKK |
| | VFQCRRATPATEAGFEDEAKTSARDAALAYHR |
| | LHQLHITGKLPAGSACSDVHMALCAVNLAECA |
| | EEKIPPSTLVEIHLTAAMGLKTRCGGKLGFLA |
| | SYFLSRAQSLCGPEHSAVPDSLRWLCHPLGQK |
| | FFMERSWSVKSAAKESLYCAQRNPADPIAQVH |
| | QAFCKNLLERAIESLVKPQAKKKAGDQEEESC |
| | EFSSALEYLKLLHSFVDSVGVMSPPLSRSSVL |
| | KSALGPDIICRWWTSAITVAISWLQGDDAAVR |
| | SHFTKVERIPKALEVTESPLVKAIFHACRAMH |
| | ASLPGKADGQQSSFCHCERASGHLWSSLNVSG |
| | ATSDPALNHVVQLLTCDLLLSLRTALWQKQAS |
| | ASQAVGETYHASGAELAGFQRDLGSLRRLAHS |
| | FRPAYRKVFLHEATVRLMAGASPTRTHQLLEH |
| | SLRRRTTQSTKHGEVDAWPGQRERATAILLAC |
| | RHLPLSFLSSPGQRAVLLAEAARTLEKVGDRR |
| | SCNDCQQMIVKLGGGTAIAAS |
| SREBF2 | MDDSGELGGLETMETLTELGDELTLGDIDEML |
| Isoform 2 | QFVSNQVGEFPDLFSEQLCSSFPGSGGSGSSS |
| (identifier: | GSSGSSSSSSNGRGSSSGAVDPSVQRSFTQVT |
| Q12772-2) | LPSFSPSAASPQAPTLQVKVSPTSVPTTPRAT |
| (SEQ ID NO: | PILQPRPQPQPQPQTQLQQQTVMITPTFSTTP |
| XX) | QTRIIQQPLIYQNAATSFQVLQPQVQSLVTSS |
| | QVQPVTIQQQVQTVQAQRVLTQTANGTLQTLA |
| | PATVQTVAAPQVQQVPVLVQPQIIKTDSLVLT |
| | TLKTDGSPVMAAVQNPALTTPIQTAALQVPTL |
| | VGSSGTILTTMPVMMGQEKVPIKQVPGGVKQL |
| | EPPKEGERRTTHNIIEKRYRSSINDKIIELKD |
| | LVMGTDAKMHKSGVLRKAIDYIKYLQQVNHKL |
| | RQENMVLKLANQKNKLLKGIDLGSLVDNEVDL |
| | KIEDFNQNVLLMSPPASDSGSQAGFSPYSIDS |
| | EPGSPLLDDAKVKDEPDSPPVALGMVDRSRIL |
| | LCVLTFLCLSFNPLTSLLQWGGAHDSDQHPHS |
| | GSGRSVLSFESGSGGWFDWMMPTLLLWLVNGV |
| | IVLSVFVKLLVHGEPVIRPHSRSSVTFWRHRK |
| | QADLDLARGVYGKKSGATHSIEEELNIHISRG |
| | TRTRTLLSSRRFCSCCRQPTNLPGSFGPGTAH |
| | LPPGPGLQPLLERDPLQPAEATPGALAAQESL |
| | PVPAGHASH |

In some aspects, the methods of the present disclosure comprise modifying the producer cells to exhibit a reduced gene and/or protein function in a cholesterol biosynthetic pathway of the producer cells. In some aspects, the methods of the present disclosure comprise culturing the producer cells, which exhibit a reduced gene and/or protein function in a cholesterol biosynthetic pathway. In some aspects, the reduced gene and/or protein in a cholesterol biosynthesis pathway comprises SREBF2 gene and/or SREBF2 protein. As used herein, the term “SREBF2 gene” refers to any transcript, genomic DNA, pre-mRNA, or mRNA. As used herein, “SREBF2 protein” refers to SREBF2 isoform 1 or SREBF2 isoform 2 disclosed above, as well as variants and mutants thereof. As used herein, the term SREBF2 protein also encompasses any fragment or variant of any of the isoforms disclosed herein that has at least one function of the wild type SREBF2 protein.

As used herein the term “reduced gene and/or protein function” refers both to reduction in physical levels (e.g., less gene sequence due to edition from the genome, or less protein due a decrease in protein expression) and to reduction in function. For example, a reduction in level of SREBF2 gene can refer to a decrease in gene function, e.g., due to the introduction of a mutation introducing a stop codon or a frame shift, to an epigenetic modification that would alter transcription, or to a mutation or other change on a promoter gene or another gene that regulates SREBF2 expression. In some aspects, a reduction in level of SREBF2 gene in a modified cell refers to a decrease in the amount (e.g., concentration) of genomic DNA, pre-mRNA, and/or mRNA that is capable of encoding a functional SREBF2 protein, e.g., wild type SREBF2 protein, compared to a reference (e.g., untreated) cell. Similarly, a reduction in SREBF2 protein can refer to changes resulting in the expression of a functional SREBF2 protein, e.g., wild type SREBF2 protein, including but not limited to changes (e.g., mutations or post-translational modifications) that cause a loss of function (partial or complete), or to the activity of molecules that bind to functional sites of SREBF2 altering, e.g., its transcriptional activating activity.

SREBF2 gene levels (e.g., presence/absence of the entire gene or a portion thereof, or gene function) can be measured by various methods known in the art. SREBF2 protein levels (e.g., presence/absence of the SREBF2 protein or fragments thereof, or quantification or protein function) can be measured by various methods known in the art.

In some aspects, the reduced expression levels of SREBF2 gene and/or expression or functional levels of SREBF2 protein in the producer cells can increase the yield of the EVs produced by the producer cells compared to EVs produced by producer cells that do not have a reduced expression levels of SREBF2 gene and/or expression levels of SREBF2 protein. In some aspects, the yield is increased at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 16 fold, at least about 17 fold, at least about 18 fold, at least about 19 fold, at least about 20 fold. at least about 21 fold, at least about 22 fold, at least about 23 fold, at least about 24 fold, at least about 25 fold, at least about 26 fold, at least about 27 fold, at least about 28 fold, at least about 29 fold, or at least about 30 fold. In some aspects, the yield is increased about 2 fold to about 30 fold, about 2 fold to about 25 fold, about 2 fold to about 20 fold, about 2 fold to about 15 fold, about 2 fold to about 10 fold, about 2 fold to about 5 fold, about 5 fold to about 30 fold, about 5 fold to about 25 fold, about 5 fold to about 20 fold, about 5 fold to about 15 fold, about 5 fold to about 10 fold, about 10 fold to about 30 fold, about 10 fold to about 25 fold, about 10 fold to about 20 fold, about 10 fold to about 15 fold, about 15 fold to about 30 fold, about 15 fold to about 25 fold, about 15 fold to about 20 fold, about 20 fold to about 30 fold, about 20 fold to about 25 fold, or about 25 fold to about 30 fold. In some aspects, the yield is increased about 2 fold, about 3 fold, about 4 fold, about 5 fold, about 6 fold, about 7 fold, about 8 fold, about 9 fold, or about 10 fold.

In some aspects, the SREBF2 gene expression is reduced by about 2 fold to about 30 fold. In some aspects, the SREBF2 gene expression is reduced about 2 fold, about 3, fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, or about 30 fold.

In some aspects, the gene and/or protein function in a cholesterol biosynthetic pathway is reduced at least about 90%, at least about 80%, at least about 70%, at least about 60%, at least about 50%, at least about 40%, at least about 30%, at least about 20%, or at least about 10%, at least about 5%, or at least about 1%. In some aspects, the gene and/or protein function in a cholesterol biosynthetic pathway is reduced at least about 90%, at least about 80%, at least about 70%, at least about 60%, at least about 50%, at least about 40%, or at least about 30%.

In some aspects, the modifying of the methods described herein comprises contacting the producer cells with an agent capable of reducing the SREBF2 gene and/or SREBF2 protein function. In some aspects, the agent comprises a Betulin (See Tang et al., Cell Metab. 2011 Jan. 5; 13(1):44-56, which is herein incorporated by reference in its entirety). In some aspects, the agent comprises Fatostatin. In some aspects, the agent comprises Pseudoprotodioscin. In some aspects, the agent comprises oligomeric amyloid β42. In some aspects, the agent comprises luteolin. In some aspects, the agent comprises clofibrate.

In some aspects, the modifying comprises contacting the producer cells with an agent capable of reducing the gene and/or protein function in a cholesterol biosynthetic pathway. In some aspects, the producer cells are modified prior to the culturing by contacting the producer cells with an agent capable of reducing the gene and/or protein function in a cholesterol biosynthetic pathway.

In some aspects, the agent capable of reducing the gene and/or protein function in a cholesterol biosynthetic pathway comprises a gene editing technology. In some aspects, the gene editing technology comprises a shRNA, siRNA, miRNA, antisense oligonucleotides, CRISPR, zinc finger nuclease, TALEN, meganuclease, restriction endonuclease, or any combination thereof. In some aspects, the gene editing technology comprises siRNA.

### IIA. Gene Editing Tools

One or more gene editing tools can be used in the methods of increasing a number of extracellular vesicles from producer cells and production and methods of producing extracellular vesicles of the present disclosure.

### II.A.1. CRISPR/Cas System

In some aspects, the gene editing tool that can be used in the present disclosure comprises a CRISPR/Cas system. Such systems can employ, for example, a Cas9 nuclease, which in some instances, is codon-optimized for the desired cell type in which it is to be expressed (e.g., CHO cells, e.g., MSC cells). CRISPR/Cas systems use Cas nucleases, e.g., Cas9 nucleases, that are targeted to a genomic site by complexing with a synthetic guide RNA (gRNA) that hybridizes to a target DNA sequence immediately preceding an NGG motif recognized by the Cas nuclease, e.g., Cas9. This results in a double-strand break three nucleotides upstream of the NGG motif. In some aspects, a modified version of a Cas nuclease (e.g., Cas9) can be used that will lead to a single stranded nick as opposed to a double stranded break. Additional fusions with other enzymes can lead to site-specific base editing in the absence of a double stranded break. A unique capability of the CRISPR/Cas9 system is the ability to simultaneously target multiple distinct genomic loci by co-expressing a single Cas9 protein with two or more gRNAs (e.g., at least one, two, three, four, five, six, seven, eight, nine or ten gRNAs). Such systems can also employ a guide RNA (gRNA) that comprises two separate molecules. In certain aspects, the two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA” or “scaffold”) molecule.

A crRNA comprises both the DNA-targeting segment (single stranded) of the gRNA and a stretch of nucleotides that forms one half of a double stranded RNA (dsRNA) duplex of the protein-binding segment of the gRNA. A corresponding tracrRNA (activator-RNA) comprises a stretch of nucleotides that forms the other half of the dsRNA duplex of the protein-binding segment of the gRNA. Thus, a stretch of nucleotides of a crRNA is complementary to and hybridizes with a stretch of nucleotides of a tracrRNA to form the dsRNA duplex of the protein-binding domain of the gRNA. As such, each crRNA can be said to have a corresponding tracrRNA. The crRNA additionally provides the single stranded DNA-targeting segment. Accordingly, a gRNA comprises a sequence that hybridizes to a target sequence and a tracrRNA. Thus, a crRNA and a tracrRNA (as a corresponding pair) hybridize to form a gRNA. If used for modification within a cell, the exact sequence and/or length of a given crRNA or tracrRNA molecule can be designed to be specific to the species in which the RNA molecules will be used (e.g., humans).

Naturally-occurring genes encoding the three elements (Cas9, tracrRNA and crRNA) are typically organized in operon(s). Naturally-occurring CRISPR RNAs differ depending on the Cas9 system and organism but often contain a targeting segment of between 21 to 72 nucleotides length, flanked by two direct repeats (DR) of a length of between 21 to 46 nucleotides (see, e.g., WO2014/131833). In the case of S. pyogenes, the DRs are 36 nucleotides long and the targeting segment is 30 nucleotides long. The 3′ located DR is complementary to and hybridizes with the corresponding tracrRNA, which in turn binds to the Cas9 protein.

Alternatively, a CRISPR system used herein can further employ a fused crRNA-tracrRNA construct (i.e., a single transcript) that functions with the codon-optimized Cas9. This single RNA is often referred to as a guide RNA or gRNA or single guide RNA, or sgRNA. Within a gRNA, the crRNA portion is identified as the “target sequence” for the given recognition site and the tracrRNA is often referred to as the “scaffold.” Briefly, a short DNA fragment containing the target sequence is inserted into a guide RNA expression plasmid. The gRNA expression plasmid comprises the target sequence (in some aspects around 20 nucleotides), a form of the tracrRNA sequence (the scaffold) as well as a suitable promoter that is active in the cell and necessary elements for proper processing in eukaryotic cells. Many of the systems rely on custom, complementary oligos that are annealed to form a double stranded DNA and then cloned into the gRNA expression plasmid.

The gRNA expression cassette and the Cas9 expression cassette are then introduced into the cell. See, for example, Mali P et al., (2013) Science 2013 Feb. 15; 339(6121):823-6; Jinek M et al., Science 2012 Aug. 17; 337(6096):816-21; Hwang W Y et al., Nat Biotechnol 2013 March; 31(3):227-9; Jiang W et al., Nat Biotechnol 2013 March; 31(3):233-9; Cronican et al., ACS Chem. Biol. 5(8):747-52 (2010); and Cong L et al., Science 2013 Feb. 15; 339(6121):819-23, each of which is herein incorporated by reference in its entirety. See also, for example, WO/2013/176772 A1, WO/2014/065596 A1, WO/2014/089290 A1, WO/2014/093622 A2, WO/2014/099750 A2, and WO/2013142578 A1, each of which is herein incorporated by reference in its entirety.

In some aspects, the Cas9 nuclease can be provided in the form of a protein. In some aspects, the Cas9 protein can be provided in the form of a complex with the gRNA. In other aspects, the Cas9 nuclease can be provided in the form of a nucleic acid encoding the protein. The nucleic acid encoding the Cas9 nuclease can be RNA (e.g., messenger RNA (mRNA)) or DNA. In some aspects, the gRNA can be provided in the form of RNA. In other aspects, the gRNA can be provided in the form of DNA encoding the RNA. In some aspects, the gRNA can be provided in the form of separate crRNA and tracrRNA molecules, or separate DNA molecules encoding the crRNA and tracrRNA, respectively.

In certain aspects, two separate Cas proteins (e.g., nickases) specific for a target site on each strand of dsDNA can create overhanging sequences complementary to overhanging sequences on another nucleic acid, or a separate region on the same nucleic acid. The overhanging ends created by contacting a nucleic acid with two nickases specific for target sites on both strands of dsDNA can be either 5′ or 3′ overhanging ends. For example, a first nickase can create a single strand break on the first strand of dsDNA, while a second nickase can create a single strand break on the second strand of dsDNA such that overhanging sequences are created. The target sites of each nickase creating the single strand break can be selected such that the overhanging end sequences created are complementary to overhanging end sequences on a different nucleic acid molecule. The complementary overhanging ends of the two different nucleic acid molecules can be annealed by the methods disclosed herein. In some aspects, the target site of the nickase on the first strand is different from the target site of the nickase on the second strand.

In some aspects, the expression of HMGCR and/or SREBF2 gene, and the HMGCR and/or SREBF2 protein encoded thereof, is reduced by contacting the cell with a CRISPR (e.g., CRISPR-Cas9 system) that is, e.g., specific to the HMGCR and/or SREBF2 gene.

In some aspects, gene editing using CRISPR reduces (e.g., HMGCR and/or SREBF2) gene levels at least about 10%, at least about 15%, at least about 20%, at least about 25%, at least about 30%, at least about 35%, at least about 40%, at least about 45%, at least about 50%, at least about 55%, at least about 60%, at least about 65%, at least 70%, at least about 75%, at least about 80%, at least about 85%, at least about 90%, at least about 95%, or about 100% with respect to HMGCR and/or SREBF2 gene levels observed in a reference cell (e.g., a corresponding cell that has not been subjected to gene editing using CRISPR). In some aspects, HMGCR and/or SREBF2 gene levels can be measured using any technique known in the art, e.g., by digital droplet PCR.

In some aspects, a nucleic acid encoding a gRNA or a Cas9 disclosed herein is an RNA or a DNA. In other aspect, the RNA or DNA encoding a gRNA or a Cas9 disclosed herein is a synthetic RNA or a synthetic DNA, respectively. In some aspects, the synthetic RNA or DNA comprises at least one unnatural nucleobase. In some aspects, all nucleobases of a certain class have been replaced with unnatural nucleobases (e.g., all uridines in a polynucleotide disclosed herein can be replaced with an unnatural nucleobase, e.g., 5-methoxyuridine or pseudouridine). In some aspects, the polynucleotide (e.g., a synthetic RNA or a synthetic DNA) comprises only natural nucleobases, i.e., A, C, T and U in the case of a synthetic DNA, or A, C, T, and U in the case of a synthetic RNA or synthetic DNA.

### II.A.2. Meganuclease

In some aspects, a gene editing tool that be used to regulate VHL expression in a cell includes a nuclease agent such as a meganuclease system. Meganucleases have been classified into four families based on conserved sequence motifs, the families are the “LAGLIDADG,” “GIY-YIG,” “H-N-H,” and “His-Cys box” families. These motifs participate in the coordination of metal ions and hydrolysis of phosphodiester bonds.

HEases are notable for their long recognition sites, and for tolerating some sequence polymorphisms in their DNA substrates. Meganuclease domains, structure and function are known, see, for example, Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764.

In some examples a naturally occurring variant, and/or engineered derivative meganuclease is used. Methods for modifying the kinetics, cofactor interactions, expression, optimal conditions, and/or recognition site specificity, and screening for activity are known, see for example, Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346; each of which is herein incorporated by reference in its entirety.

Any meganuclease can be used herein, including, but not limited to, I-SceI, I-SceII, I-SceIII, I-SceIV, I-SceV, I-SecVI, I-SceVII, I-CeuI, I-CeuAIIP, I-CreI, I-CrepsbIP, I-CrepsbIIP, I-CrepsbIIIP, I-CrepsbIVP, I-TliI, I-PpoI, PI-PspI, F-SceI, F-SceII, F-SuvI, F-TevI, F-TevII, I-AmaI, I-Anil, I-ChuI, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-Njal, I-Nsp236IP, I-PakI, I-PboIP, I-PcuIP, I-PcuAI, I-PcuVI, I-PgrIP, I-PobIP, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP, PI-MtuHIIP, PI-PfuI, PI-PfuII, PI-PkoI, PI-PkoII, PI-Rma43812IP, PI-SpBetaIP, PI-SceI, PI-TfuI, PI-TfuII, PI-ThyI, PI-TliI, PI-TliII, or any active variants or fragments thereof.

In some aspects, the meganuclease recognizes double-stranded DNA sequences of 12 to 40 base pairs. In some aspects, the meganuclease recognizes one perfectly matched target sequence in the genome. In some aspects, the meganuclease is a homing nuclease. In some aspects, the homing nuclease is a “LAGLIDADG” family of homing nuclease. In some aspects, the “LAGLIDADG” family of homing nuclease is selected from I-SceI, I-CreI, I-Dmol, or combinations thereof.

### II.A.3 TALEN

In some aspects, the gene editing tool that can be used in the present disclosure comprises a nuclease agent, such as a Transcription Activator-Like Effector Nuclease (TALEN). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI.

The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al., (2010) PNAS 10.1073/pnas.1013133107; Scholze & Boch (2010) Virulence 1:428-432; Christian et al., Genetics (2010) 186:757-761; Li et al., (2010) Nuc. Acids Res. (2010) doi:10.1093/nar/gkg704; and Miller et al., (2011) Nature Biotechnology 29:143-148; all of which are herein incorporated by reference in their entirety.

Non-limiting examples of suitable TAL nucleases, and methods for preparing suitable TAL nucleases, are disclosed, e.g., in US Patent Application Nos. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1, each of which is herein incorporated by reference in its entirety.

In various aspects, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors as described herein.

### II.A.4 Zinc Finger Nuclease (ZFN)

In some aspects, the gene editing tool that can be used in the present disclosure comprises a nuclease agent, such as a zinc-finger nuclease (ZFN) system. Zinc finger-based systems comprise a fusion protein comprising two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired, one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

In some aspects, a zinc finger binding domain comprises one or more zinc fingers. See, e.g., Miller et al., (1985) EMBO J 4:1609-1614; Rhodes (1993) Scientific American February:56-65; U.S. Pat. No. 6,453,242; each of which is herein incorporated by reference in its entirety. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore, the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some aspects, the DNA-binding domains of individual ZFNs comprise between three and six individual zinc finger repeats and can each recognize between 9 and 18 basepairs.

Zinc finger binding domains can be engineered to bind to a sequence of choice. See, for example, Beerli et al., (2002) Nature Biotechnol. 20:135-141; Pabo et al., (2001) Ann. Rev. Biochem. 70:313-340; Isalan et al., (2001) Nature Biotechnol. 19:656-660; Segal et al., (2001) Curr. Opin. Biotechnol. 12:632-637; Choo et al., (2000) Curr. Opin. Struct. Biol. 10:411-416; 2002-2003 Catalogue, New England Biolabs, Beverly, Mass.; and Belfort et al., (1997) Nucleic Acids Res. 25:3379-3388; each of which is herein incorporated by reference in its entirety. An engineered zinc finger binding domain can have a novel binding specificity, compared to a naturally-occurring zinc finger protein. Engineering methods include, but are not limited to, rational design and various types of selection.

Selection of a target DNA sequence for binding by a zinc finger domain can be accomplished, for example, according to the methods disclosed in U.S. Pat. No. 6,453,242. It will be clear to those skilled in the art that simple visual inspection of a nucleotide sequence can also be used for selection of a target DNA sequence. Accordingly, any means for target DNA sequence selection can be used in the methods described herein. A target site generally has a length of at least 9 nucleotides and, accordingly, is bound by a zinc finger binding domain comprising at least three zinc fingers. However, binding of, for example, a 4-finger binding domain to a 12-nucleotide target site, a 5-finger binding domain to a 15-nucleotide target site or a 6-finger binding domain to an 18-nucleotide target site, is also possible. As will be apparent, binding of larger binding domains (e.g., 7-, 8-, 9-finger and more) to longer target sites is also possible.

The enzymatic domain portion of the zinc finger fusion proteins can be obtained from any endo- or exonuclease. Exemplary endonucleases from which an enzymatic domain can be derived include, but are not limited to, restriction endonucleases and homing endonucleases. See, for example, 2002-2003 Catalogue, New England Biolabs, Beverly, Mass.; and Belfort et al., (1997) Nucleic Acids Res. 25:3379-3388. Additional enzymes which cleave DNA are known (e.g., 51 Nuclease; mung bean nuclease; pancreatic DNaseI; micrococcal nuclease; yeast HO endonuclease; see also Linn et al., (eds.) Nucleases, Cold Spring Harbor Laboratory Press, 1993). One or more of these enzymes (or functional fragments thereof) can be used as a source of cleavage domains.

Exemplary restriction endonucleases (restriction enzymes) suitable for use as an enzymatic domain of the ZFPs described herein are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al., (1992) Proc. Natl. Acad. Sci. USA 89:4275-4279; Li et al., (1993) Proc. Natl. Acad. Sci. USA 90:2764-2768; Kim et al., (1994a) Proc. Natl. Acad. Sci. USA 91:883-887; Kim et al., (1994b) J. Biol. Chem. 269: 31,978-31,982, each of which is herein incorporated by reference in its entirety.

### II.A.5 Interference RNA (RNAi)

In some aspects, a gene editing tool that can be used to reduce the expression of VHL in a cell includes an RNA interference molecule (“RNAi”). As used herein, RNAi are RNA polynucleotide that mediates the decreased the expression of an endogenous target gene product by degradation of a target mRNA through endogenous gene silencing pathways (e.g., Dicer and RNA-induced silencing complex (RISC)). Non-limiting examples of RNAi agents include micro RNAs (also referred to herein as “miRNAs”), short hair-pin RNAs (shRNAs), small interfering RNAs (siRNAs), RNA aptamers, or combinations thereof.

In some aspects, the gene editing tools useful for the present disclosure comprises one or more miRNAs. “miRNAs” refer to naturally occurring, small non-coding RNA molecules of about 21-25 nucleotides in length. In some aspects, the miRNAs useful for the present disclosure are at least partially complementary to a gene in a cholesterol synthesis pathway, e.g., HMGCR and/or SREBF2 mRNA molecule. miRNAs can downregulate (e.g., decrease) expression of an endogenous target gene product (i.e., HMGCR and/or SREBF2 protein) through translational repression, cleavage of the mRNA, and/or deadenylation.

In some aspects, a gene editing tool that can be used with the present disclosure comprises one or more shRNAs. “shRNAs” (or “short hairpin RNA” molecules) refer to an RNA sequence comprising a double-stranded region and a loop region at one end forming a hairpin loop, which can be used to reduce and/or silence a gene expression. The double-stranded region is typically about 19 nucleotides to about 29 nucleotides in length on each side of the stem, and the loop region is typically about three to about ten nucleotides in length (and 3′- or 5′-terminal single-stranded overhanging nucleotides are optional). shRNAs can be cloned into plasmids or in non-replicating recombinant viral vectors to be introduced intracellularly and result in the integration of the shRNA-encoding sequence into the genome. As such, an shRNA can provide stable and consistent repression of endogenous target gene translation and expression.

In some aspects, a gene editing tool useful for the present disclosure comprises one or more siRNAs. “siRNAs” refer to double stranded RNA molecules typically about 21-23 nucleotides in length. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. In certain aspects, an siRNA is about 18, about 19, about 20, about 21, about 22, about 23, or about 24 nucleotides in length and has a 2 base overhang at its 3′ end. siRNAs and shRNAs are further described in Fire et al., Nature 391:19, 1998 and U.S. Pat. Nos. 7,732,417; 8,202,846; and 8,383,599; each of which is herein incorporated by reference in its entirety.

### II.A.6 Antisense Oligonucleotides (ASO)

In some aspects, a gene editing tool that can be used to reduce the expression of HMGCR and/or SREBF2 gene and/or HMGCR and/or SREBF2 protein in a cell includes antisense oligonucleotides. As used herein, “antisense oligonucleotide” or “ASO” refer to an oligonucleotide capable of modulating expression of a target gene (i.e., HMGCR and/or SREBF2) by hybridizing to a target nucleic acid, in particular to a contiguous sequence on a target nucleic acid. Antisense oligonucleotides are not essentially double stranded and are therefore not siRNAs or shRNAs.

In some aspects, ASOs useful for the present disclosure are single stranded. It is understood that single stranded oligonucleotides of the present disclosure can form hairpins or intermolecular duplex structures (duplex between two molecules of the same oligonucleotide), as long as the degree of intra or inter self-complementarity is less than approximately 50% across of the full length of the oligonucleotide. In some aspects, ASOs useful for the present disclosure can comprise one or more modified nucleosides or nucleotides, such as 2′ sugar modified nucleosides. Additional modifications that can be made to an ASO (e.g., such as those that can be used to inhibit or reduce HMGCR and/or SREBF2 gene expression) are provided in, e.g., US Publ. No. 2019/0275148 A1.

In some aspects, ASOs can reduce the expression of HMGCR and/or SREBF2 protein via nuclease mediated degradation of the HMGCR and/or SREBF2 transcript (e.g., mRNA), where the ASOs are capable of recruiting a nuclease, e.g., RNase H, such as RNaseH1. RNase H is a ubiquitous enzyme that hydrolyzes the RNA strand of an RNA/DNA duplex. Accordingly, in certain aspects, once bound to the target sequence (e.g., HMGCR and/or SREBF2 mRNA), ASOs can induce the degradation of the HMGCR and/or SREBF2 mRNA and thereby, reduce the expression of HMGCR and/or SREBF2 protein.

As disclosed herein, the above examples of gene editing tools are not intended to be limiting and any gene editing tool available in the art can be used to reduce or inhibit the expression of HMGCR and/or SREBF2 gene and/or HMGCR and/or SREBF2 protein.

### IIB. Protein Inhibitors

One or more protein inhibitors (e.g., small molecule HMGCR and/or SREBF2 inhibitors) can be used in the methods of increasing a number of extracellular vesicles produced from producer cells and methods of producing extracellular vesicles of the present disclosure.

In some aspects, the protein inhibitor tool that can be used in the present disclosure comprises a proteolysis targeting chimera (PROTAC). PROTACs are heterobifunctional small molecules with three chemical elements: a ligand binding to a target protein, a ligand binding to E3 ubiquitin ligase, and a linker for conjugating these two ligands. PROTAC is a chemical knockdown strategy that degrades the target protein through the ubiquitin-proteasome system. Different from the competitive- and occupancy-driven process of traditional inhibitors, PROTACs are catalytic in their mode of action, which can promote target protein degradation at low exposures. Moreover, traditional small molecule inhibitors usually inhibit the enzymatic activity of the target, while PROTACs affect not only the enzymatic activity of the protein but also non-enzymatic activity by degrading the entire protein.

In some aspects, the PROTAC comprises a statin (e.g., an HMGCR ligand) linked to E3 ubiquitin ligase ligand (e.g., VHL, CRBN, cIAPs, and MDM2), as described in Luo et al., Acta Pharmaceutica Sinica B, 2021; 11(5):1300-1314, which is herein incorporated by reference in its entirety. In some aspects, the PROTAC degrades HMCGR as described in Li et al., J. Med. Chem. 2020, 63, 9, 4908-4928, which is herein incorporated by reference in its entirety.

In some aspects, the protein inhibitor comprises cariprazine.

### III. Extracellular Vesicles, e.g., Exosomes

Disclosed herein are methods of producing EVs, e.g., exosomes. The methods improve EV, e.g., exosome production yields.

In some aspects, the EVs produced by the producer cells (e.g., the methods of increasing EV production and methods of producing EVs described herein) have decreased cholesterol content per EV compared to EVs produced by producer cells that the gene and/or protein function in a cholesterol biosynthetic pathway is not reduced. In some aspects, the cholesterol content per EV is reduced by about 10%, about 20%, about 30%, about 40%, about 50%, about 60%, about 70%, or about 80%. In some aspects, the cholesterol content per EV is reduced by about 1% to about 80%, about 10% to about 80%, about 10% to about 70%, about 10% to about 60%, about 10% to about 50%, about 10% to about 40%, about 10% to about 30%, about 10% to about 20%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 30% to about 40%, about 40% to about 80%, about 40% to about 70%, about 40% to about 60%, or about 40% to about 50%.

In some aspects, the EVs, e.g., exosomes, described herein are extracellular vesicles with a diameter between about 20-300 nm. In some aspects, the size of the EVs obtained from the producer cells described herein (e.g., having a reduced gene and/or protein function in a cholesterol biosynthetic pathway) do not have a difference in average size compared to EVs produced from producer cells that do not have a reduced gene and/or protein function in a cholesterol biosynthetic pathway. In certain aspects, an EV, e.g., exosome, of the present disclosure has a diameter between about 20-290 nm, 20-280 nm, 20-270 nm, 20-260 nm, 20-250 nm, 20-240 nm, 20-230 nm, 20-220 nm, 20-210 nm, 20-200 nm, 20-190 nm, 20-180 nm, 20-170 nm, 20-160 nm, 20-150 nm, 20-140 nm, 20-130 nm, 20-120 nm, 20-110 nm, 20-100 nm, 20-90 nm, 20-80 nm, 20-70 nm, 20-60 nm, 20-50 nm, 20-40 nm, 20-30 nm, 30-300 nm, 30-290 nm, 30-280 nm, 30-270 nm, 30-260 nm, 30-250 nm, 30-240 nm, 30-230 nm, 30-220 nm, 30-210 nm, 30-200 nm, 30-190 nm, 30-180 nm, 30-170 nm, 30-160 nm, 30-150 nm, 30-140 nm, 30-130 nm, 30-120 nm, 30-110 nm, 30-100 nm, 30-90 nm, 30-80 nm, 30-70 nm, 30-60 nm, 30-50 nm, 30-40 nm, 40-300 nm, 40-290 nm, 40-280 nm, 40-270 nm, 40-260 nm, 40-250 nm, 40-240 nm, 40-230 nm, 40-220 nm, 40-210 nm, 40-200 nm, 40-190 nm, 40-180 nm, 40-170 nm, 40-160 nm, 40-150 nm, 40-140 nm, 40-130 nm, 40-120 nm, 40-110 nm, 40-100 nm, 40-90 nm, 40-80 nm, 40-70 nm, 40-60 nm, 40-50 nm, 50-300 nm, 50-290 nm, 50-280 nm, 50-270 nm, 50-260 nm, 50-250 nm, 50-240 nm, 50-230 nm, 50-220 nm, 50-210 nm, 50-200 nm, 50-190 nm, 50-180 nm, 50-170 nm, 50-160 nm, 50-150 nm, 50-140 nm, 50-130 nm, 50-120 nm, 50-110 nm, 50-100 nm, 50-90 nm, 50-80 nm, 50-70 nm, 50-60 nm, 60-300 nm, 60-290 nm, 60-280 nm, 60-270 nm, 60-260 nm, 60-250 nm, 60-240 nm, 60-230 nm, 60-220 nm, 60-210 nm, 60-200 nm, 60-190 nm, 60-180 nm, 60-170 nm, 60-160 nm, 60-150 nm, 60-140 nm, 60-130 nm, 60-120 nm, 60-110 nm, 60-100 nm, 60-90 nm, 60-80 nm, 60-70 nm, 70-300 nm, 70-290 nm, 70-280 nm, 70-270 nm, 70-260 nm, 70-250 nm, 70-240 nm, 70-230 nm, 70-220 nm, 70-210 nm, 70-200 nm, 70-190 nm, 70-180 nm, 70-170 nm, 70-160 nm, 70-150 nm, 70-140 nm, 70-130 nm, 70-120 nm, 70-110 nm, 70-100 nm, 70-90 nm, 70-80 nm, 80-300 nm, 80-290 nm, 80-280 nm, 80-270 nm, 80-260 nm, 80-250 nm, 80-240 nm, 80-230 nm, 80-220 nm, 80-210 nm, 80-200 nm, 80-190 nm, 80-180 nm, 80-170 nm, 80-160 nm, 80-150 nm, 80-140 nm, 80-130 nm, 80-120 nm, 80-110 nm, 80-100 nm, 80-90 nm, 90-300 nm, 90-290 nm, 90-280 nm, 90-270 nm, 90-260 nm, 90-250 nm, 90-240 nm, 90-230 nm, 90-220 nm, 90-210 nm, 90-200 nm, 90-190 nm, 90-180 nm, 90-170 nm, 90-160 nm, 90-150 nm, 90-140 nm, 90-130 nm, 90-120 nm, 90-110 nm, 90-100 nm, 100-300 nm, 110-290 nm, 120-280 nm, 130-270 nm, 140-260 nm, 150-250 nm, 160-240 nm, 170-230 nm, 180-220 nm, or 190-210 nm. The size of the EV, e.g., exosome, described herein can be measured according to methods known in the art.

In some aspects, an EV, e.g., exosome, of the present disclosure comprises a bi-lipid membrane (“EV, e.g., exosome, membrane”), comprising an interior (luminal) surface and an exterior surface. In certain aspects, the interior (luminal) surface faces the inner core (i.e., lumen) of the EV, e.g., exosome. In certain aspects, the exterior surface can be in contact with the endosome, the multivesicular bodies, or the membrane/cytoplasm of a producer cell or a target cell.

In some aspects, the EV, e.g., exosome, membrane comprises lipids and fatty acids. In some aspects, the EV, e.g., exosome, membrane comprises phospholipids, glycolipids, fatty acids, sphingolipids, phosphoglycerides, sterols, cholesterols, and phosphatidylserines.

In some aspects, the EV, e.g., exosome, membrane comprises an inner leaflet and an outer leaflet. The composition of the inner and outer leaflet can be determined by transbilayer distribution assays known in the art, see, e.g., Kuypers et al., Biohim Biophys Acta 1985 819:170. In some aspects, the composition of the outer leaflet is between approximately 70-90% choline phospholipids, between approximately 0-15% acidic phospholipids, and between approximately 5-30% phosphatidylethanolamine. In some aspects, the composition of the inner leaflet is between approximately 15-40% choline phospholipids, between approximately 10-50% acidic phospholipids, and between approximately 30-60% phosphatidylethanolamine. In some aspects, the EV, e.g., exosome comprises between about 40% to about 60% cholesterol. In some aspects, the EVs produced by the producer cells have a decreased cholesterol content per EV compared to EVs produced by producer cells that the gene and/or protein function in a cholesterol biosynthetic pathway is not reduced.

In some aspects, the EV, e.g., exosome, membrane comprises one or more polysaccharide, such as glycan.

In some aspects, the EV, e.g., exosome, of the present disclosure comprises an IL-12 moiety, wherein the IL-12 moiety is linked to the EV via a scaffold moiety, either on the exterior surface of the EV or on the luminal surface of the EV.

In some aspects, the EV, e.g., exosome, of the present disclosure comprises an IL-12 moiety in the lumen of the EV. In other aspects, the EV comprises an IL-12 moiety on the exterior surface of the EV, optionally linked via a first scaffold moiety (e.g., Scaffold X). In other aspects, the EV comprises an IL-12 moiety on the luminal surface of the EV, optionally linked via a scaffold moiety (e.g., Scaffold X or Scaffold Y).

### III.A. Scaffold Moieties

One or more scaffold moieties can be used to anchor an IL-12 moiety to the EV of the present disclosure. In some aspects, the IL-12 moiety is linked to the scaffold moiety. In some aspects, the EV comprises more than one scaffold moiety. In some aspects, the IL-12 is linked to a first scaffold moiety and a second moiety (e.g., a second polypeptide or a polynucleotide) is linked to a second scaffold moiety. In some aspects, the first scaffold moiety and the second scaffold moiety are the same type of scaffold moiety, e.g., the first and second scaffold moieties are both a Scaffold X protein. In some aspects, the first scaffold moiety and the second scaffold moiety are different types of scaffold moiety, e.g., the first scaffold moiety is a Scaffold Y protein and the second scaffold moiety is a Scaffold X protein. In some aspects, the first scaffold moiety is a Scaffold Y, disclosed herein. In some aspects, the first scaffold moiety is a Scaffold X, disclosed herein. In some aspects, the second scaffold moiety is a Scaffold Y, disclosed herein. In some aspects, the second scaffold moiety is a Scaffold X, disclosed herein.

In some aspects, the EV comprises one or more scaffold moieties, which are capable of anchoring, e.g., n IL-12 moiety, to the EV, e.g., exosome, (e.g., either on the luminal surface or on the exterior surface). In certain aspects, the scaffold moiety is a polypeptide (“scaffold protein”). In certain aspects, the scaffold protein comprises an exosome protein or a fragment thereof. In other aspects, scaffold moieties are non-polypeptide moieties. In some aspects, scaffold proteins include various membrane proteins, such as transmembrane proteins, integral proteins and peripheral proteins, enriched on the exosome membranes. They can include various CD proteins, transporters, integrins, lectins, and cadherins. In certain aspects, a scaffold moiety (e.g., scaffold protein) comprises Scaffold X. In other aspects, a scaffold moiety (e.g., exosome protein) comprises Scaffold Y. In further aspects, a scaffold moiety (e.g., exosome protein) comprises both a Scaffold X and a Scaffold Y.

In some aspects, the IL-12 moiety is linked to a scaffold moiety (e.g., Scaffold X) on the exterior surface of the EV. In some aspects, the IL-12 moiety is linked to a scaffold moiety (e.g., Scaffold X) on the luminal surface of the EV. In some aspects, the IL-12 moiety is linked to a scaffold moiety (e.g., Scaffold Y) on the luminal surface of the EV.

### III.A.1. Scaffold X-Engineered EVs, e.g., Exosomes

In some aspects, EVs, e.g., exosomes, of the present disclosure comprise a membrane modified in its composition. For example, their membrane compositions can be modified by changing the protein, lipid, or glycan content of the membrane.

In some aspects, the surface-engineered EVs, e.g., exosomes, are generated by chemical and/or physical methods, such as PEG-induced fusion and/or ultrasonic fusion. In other aspects, the surface-engineered EVs, e.g., exosomes, are generated by genetic engineering. EVs, e.g., exosomes, produced from a genetically-modified producer cell or a progeny of the genetically-modified cell can contain modified membrane compositions. In some aspects, surface-engineered EVs, e.g., exosomes, have scaffold moiety (e.g., exosome protein, e.g., Scaffold X) at a higher or lower density (e.g., higher number) or include a variant or a fragment of the scaffold moiety.

For example, surface (e.g., Scaffold X)-engineered EVs, can be produced from a cell (e.g., HEK293 cells) transformed with an exogenous sequence encoding a scaffold moiety (e.g., exosome proteins, e.g., Scaffold X) or a variant or a fragment thereof. EVs including scaffold moiety expressed from the exogenous sequence can include modified membrane compositions.

Various modifications or fragments of the scaffold moiety can be used for the aspects of the present disclosure. For example, scaffold moiety modified to have enhanced affinity to a binding agent can be used for generating surface-engineered EV that can be purified using the binding agent. Scaffold moieties modified to be more effectively targeted to EVs and/or membranes can be used. Scaffold moieties modified to comprise a minimal fragment required for specific and effective targeting to exosome membranes can be also used.

Scaffold moieties can be engineered to be expressed as a fusion molecule, e.g., fusion molecule of Scaffold X to an IL-12 moiety. For example, the fusion molecule can comprise a scaffold moiety disclosed herein (e.g., Scaffold X, e.g., PTGFRN, BSG, IGSF2, IGSF3, IGSF8, ITGB1, ITGA4, SLC3A2, ATP transporter, or a fragment or a variant thereof) linked to an IL-12 moiety.

In some aspects, the surface (e.g., Scaffold X)-engineered EVs described herein demonstrate superior characteristics compared to EVs known in the art. For example, surface (e.g., Scaffold X)-engineered contain modified proteins more highly enriched on their surface than naturally occurring EVs or the EVs produced using conventional exosome proteins. Moreover, the surface (e.g., Scaffold X)-engineered EVs of the present disclosure can have greater, more specific, or more controlled biological activity compared to naturally occurring EVs or the EVs produced using conventional exosome proteins.

In some aspects, the Scaffold X comprises Prostaglandin F2 receptor negative regulator (the PTGFRN polypeptide). The PTGFRN protein can be also referred to as CD9 partner 1 (CD9P-1), Glu-Trp-Ile EWI motif-containing protein F (EWI-F), Prostaglandin F2-alpha receptor regulatory protein, Prostaglandin F2-alpha receptor-associated protein, or CD315. The full length amino acid sequence of the human PTGFRN protein (Uniprot Accession No. Q9P2B2) is shown at Table 3 as SEQ ID NO: 1. The PTGFRN polypeptide contains a signal peptide (amino acids 1 to 25 of SEQ ID NO: 1), the extracellular domain (amino acids 26 to 832 of SEQ ID NO: 1), a transmembrane domain (amino acids 833 to 853 of SEQ ID NO: 1), and a cytoplasmic domain (amino acids 854 to 879 of SEQ ID NO: 1). The mature PTGFRN polypeptide consists of SEQ ID NO: 1 without the signal peptide, i.e., amino acids 26 to 879 of SEQ ID NO: 1. In some aspects, a PTGFRN polypeptide fragment useful for the present disclosure comprises a transmembrane domain of the PTGFRN polypeptide. In other aspects, a PTGFRN polypeptide fragment useful for the present disclosure comprises the transmembrane domain of the PTGFRN polypeptide and (i) at least five, at least 10, at least 15, at least 20, at least 25, at least 30, at least 40, at least 50, at least 70, at least 80, at least 90, at least 100, at least 110, at least 120, at least 130, at least 140, at least 150 amino acids at the N terminus of the transmembrane domain, (ii) at least five, at least 10, at least 15, at least 20, or at least 25 amino acids at the C terminus of the transmembrane domain, or both (i) and (ii).

In some aspects, the fragments of PTGFRN polypeptide lack one or more functional or structural domains, such as IgV.

In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 26 to 879 of SEQ ID NO: 1. In other aspects, the Scaffold X comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 1. In other aspects, the Scaffold X comprises an amino acid sequence at least about at least about 70%, at least about 75%, at least about 80%, at least about 85%, at least about 90%, at least about 95%, at least about 96%, at least about 97%, at least about 98%, at least about 99%, or about 100% identical to SEQ ID NO: 33. In other aspects, the Scaffold X comprises the amino acid sequence of SEQ ID NO: 33, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof. In some aspects, the Scaffold X comprises the amino acid sequence of SEQ ID NO: 33 and 1 amino acid, two amino acids, three amino acids, four amino acids, five amino acids, six amino acids, seven amino acids, eight amino acids, nine amino acids, ten amino acids, 11 amino acids, 12 amino acids, 13 amino acids, 14 amino acids, 15 amino acids, 16 amino acids, 17 amino acids, 18 amino acids, 19 amino acids, or 20 amino acids or longer at the N terminus and/or C terminus of SEQ ID NO: 33.

TABLE 3
Exemplary Scaffold X Protein Sequences.

| Protein | Sequence |
|---|---|
| The | MGRLASRPLLLALLSLALCRGRVVRVPTATLVRVVG |
| PTGFRN | TELVIPCNVSDYDGPSEQNFDWSFSSLGSSFVELAS |
| Protein | TWEVGFPAQLYQERLQRGEILLRRTANDAVELHIKN |
| (SEQ ID | VQPSDQGHYKCSTPSTDATVQGNYEDTVQVKVLADS |
| NO: 1) | LHVGPSARPPPSLSLREGEPFELRCTAASASPLHTH |
| | LALLWEVHRGPARRSVLALTHEGRFHPGLGYEQRYH |
| | SGDVRLDTVGSDAYRLSVSRALSADQGSYRCIVSEW |
| | IAEQGNWQEIQEKAVEVATVVIQPSVLRAAVPKNVS |
| | VAEGKELDLTCNITTDRADDVRPEVTWSFSRMPDST |
| | LPGSRVLARLDRDSLVHSSPHVALSHVDARSYHLLV |
| | RDVSKENSGYYYCHVSLWAPGHNRSWHKVAEAVSSP |
| | AGVGVTWLEPDYQVYLNASKVPGFADDPTELACRVV |
| | DTKSGEANVRFTVSWYYRMNRRSDNVVTSELLAVMD |
| | GDWTLKYGERSKQRAQDGDFIFSKEHTDTFNFRIQR |
| | TTEEDRGNYYCVVSAWTKQRNNSWVKSKDVFSKPVN |
| | IFWALEDSVLVVKARQPKPFFAAGNTFEMTCKVSSK |
| | NIKSPRYSVLIMAEKPVGDLSSPNETKYIISLDQDS |
| | VVKLENWTDASRVDGVVLEKVQEDEFRYRMYQTQVS |
| | DAGLYRCMVTAWSPVRGSLWREAATSLSNPIEIDFQ |
| | TSGPIFNASVHSDTPSVIRGDLIKLFCIITVEGAAL |
| | DPDDMAFDVSWFAVHSFGLDKAPVLLSSLDRKGIVT |
| | TSRRDWKSDLSLERVSVLEFLLQVHGSEDQDFGNYY |
| | CSVTPWVKSPTGSWQKEAEIHSKPVFITVKMDVLNA |
| | FKYPLLIGVGLSTVIGLLSCLIGYCSSHWCCKKEVQ |
| | ETRRERRRLMSMEMD |
| The | GPIFNASVHSDTPSVIRGDLIKLFCIITVEGAALDP |
| PTGFRN | DDMAFDVSWFAVHSFGLDKAPVLLSSLDRKGIVTTS |
| protein | RRDWKSDLSLERVSVLEFLLQVHGSEDQDFGNYYCS |
| Fragment | VTPWVKSPTGSWQKEAEIHSKPVFITVKMDVLNAFK |
| (SEQ ID | YPLLIGVGLSTVIGLLSCLIGYCSSHWCCKKEVQET |
| NO: 33) | RRERRRLMSMEM |
| | 687-878 of SEQ ID NO: 1 |

Non-limiting examples of other Scaffold X proteins can be found at U.S. Pat. No. 10,195,290B1, issued Feb. 5, 2019, which is incorporated by reference in its entireties.

In some aspects, the sequence encodes a fragment of the scaffold moiety lacking at least 5, 10, 50, 100, 200, 300, 400, 500, 600, 700, or 800 amino acids from the N-terminus of the native protein. In some aspects, the sequence encodes a fragment of the scaffold moiety lacking at least 5, 10, 50, 100, 200, 300, 400, 500, 600, 700, or 800 amino acids from the C-terminus of the native protein. In some aspects, the sequence encodes a fragment of the scaffold moiety lacking at least 5, 10, 50, 100, 200, 300, 400, 500, 600, 700, or 800 amino acids from both the N-terminus and C-terminus of the native protein. In some aspects, the sequence encodes a fragment of the scaffold moiety lacking one or more functional or structural domains of the native protein.

In some aspects, the scaffold moieties, e.g., Scaffold X, e.g., a PTGFRN protein, are linked to one or more heterologous proteins. The one or more heterologous proteins can be linked to the N-terminus of the scaffold moieties. The one or more heterologous proteins can be linked to the C-terminus of the scaffold moieties. In some aspects, the one or more heterologous proteins are linked to both the N-terminus and the C-terminus of the scaffold moieties. In some aspects, the heterologous protein is a mammalian protein. In some aspects, the heterologous protein is a human protein.

In some aspects, Scaffold X can be used to link any moiety, e.g., an IL-12 moiety, to the luminal surface and on the exterior surface of the EV, e.g., exosome, at the same time. For example, the PTGFRN polypeptide can be used to link an IL-12 moiety inside the lumen (e.g., on the luminal surface) in addition to the exterior surface of the EV, e.g., exosome. In some aspects, Scaffold X is a scaffold protein that is capable of anchoring the IL-12 on the luminal surface of the EV and/or on the exterior surface of the EV.

### III.A.2. Scaffold Y-Engineered EVs, e.g., Exosomes

In some aspects, EVs, e.g., exosomes, of the present disclosure comprise an internal space (i.e., lumen) that is different from that of the naturally occurring EVs. For example, the EV can be changed such that the composition in the luminal surface of the EV, e.g., exosome has the protein, lipid, or glycan content different from that of the naturally-occurring exosomes.

In some aspects, engineered EVs, e.g., exosomes, can be produced from a cell transformed with an exogenous sequence encoding a scaffold moiety (e.g., exosome proteins, e.g., Scaffold Y) or a modification or a fragment of the scaffold moiety that changes the composition or content of the luminal surface of the EV, e.g., exosome. Various modifications or fragments of the exosome protein that can be expressed on the luminal surface of the EV, e.g., exosome, can be used for the aspects of the present disclosure.

In some aspects, the exosome proteins that can change the luminal surface of the EVs, e.g., exosomes, include, but are not limited to, the myristoylated alanine rich Protein Kinase C substrate (MARCKS) protein, the myristoylated alanine rich Protein Kinase C substrate like 1 (MARCKSL1) protein, the brain acid soluble protein 1 (BASP1) protein, or any combination thereof.

In some aspects, Scaffold Y comprises the MARCKS protein (Uniprot accession no. P29966). The MARCKS protein is also known as protein kinase C substrate, 80 kDa protein, light chain. The full-length human MARCKS protein is 332 amino acids in length and comprises a calmodulin-binding domain at amino acid residues 152-176. In some aspects, Scaffold Y comprises the MARCKSL1 protein (Uniprot accession no. P49006). The MARCKSL1 protein is also known as MARCKS-like protein 1, and macrophage myristoylated alanine-rich C kinase substrate. The full-length human MARCKSL1 protein is 195 amino acids in length. The MARCKSL1 protein has an effector domain involved in lipid-binding and calmodulin-binding at amino acid residues 87-110. In some aspects, the Scaffold Y comprises the BASP1 protein (Uniprot accession number P80723). The BASP1 protein is also known as 22 kDa neuronal tissue-enriched acidic protein or neuronal axonal membrane protein NAP-22. The full-length human BASP1 protein sequence (isomer 1) is 227 amino acids in length. An isomer produced by an alternative splicing is missing amino acids 88 to 141 from SEQ ID NO: 49 (isomer 1). Table 4 provides the full-length sequences for the exemplary Scaffold Y disclosed herein (i.e., the MARCKS, MARCKSL1, and BASP1 proteins).

TABLE 4
Exemplary Scaffold Y Protein Sequences.

| Protein | Sequence |
|---|---|
| The BASP1 | MGGKLSKKKK GYNVNDEKAK EKDKKAEGAA |
| protein | TEEEGTPKES EPQAAAEPAE AKEGKEKPDQ |
| (SEQ ID | DAEGKAEEKE GEKDAAAAKE EAPKAEPEKT |
| NO: 49) | EGAAEAKAEP PKAPEQEQAA PGPAAGGEAP |
| | KAAEAAAAPA ESAAPAAGEE PSKEEGEPKK |
| | TEAPAAPAAQ ETKSDGAPAS DSKPGSSEAA |
| | PSSKETPAAT EAPSSTPKAQ GPAASAEEPK |
| | PVEAPAANSD QTVTVKE |

The mature BASP1 protein sequence is missing the first Met from SEQ ID NO: 49 and thus contains amino acids 2 to 227 of SEQ ID NO: 49.

In other aspects, Scaffold Y useful for the present disclosure comprises an amino acid sequence at least about 70%, at least about 75%, at least about 80%, at least about 85%, at least about 90%, at least about 95%, at least about 96%, at least about 97%, at least about 98%, at least about 99%, or about 100% identical to amino acids 2 to 227 of SEQ ID NO: 49. In other aspects, a Scaffold Y useful for the present disclosure comprises the amino acid sequence of SEQ ID NO: 49, except one amino acid mutation, two amino acid mutations, three amino acid mutations, four amino acid mutations, five amino acid mutations, six amino acid mutations, or seven amino acid mutations. The mutations can be a substitution, an insertion, a deletion, or any combination thereof.

In some aspects, the protein sequence of SEQ ID NO: 49 without Met at amino acid residue 1 of the SEQ ID NO: 49 is sufficient to be a Scaffold Y for the present disclosure (e.g., scaffold moiety linked to an IL-12 moiety. In some aspects, the Scaffold Y comprises an amino acid sequence at least about 70%, at least about 75%, at least about 80%, at least about 85%, at least about 90%, at least about 95%, at least about 96%, at least about 97%, at least about 98%, at least about 99%, or about 100% identical to SEQ ID NO: 49 without Met at amino acid residue 1 of the SEQ ID NO: 49.

Scaffold Y-engineered EVs, e.g., exosomes described herein can be produced from a cell transformed with a sequence set forth in SEQ ID NO: 49 without Met at amino acid residue 1 of the SEQ ID NO: 49.

In some aspects, the Scaffold Y protein useful for the present disclosure comprises an “N-terminus domain” (ND) and an “effector domain” (ED), wherein the ND and/or the ED are associated with the luminal surface of the EV, e.g., an exosome. In some aspects, the Scaffold Y protein useful for the present disclosure comprises an intracellular domain, a transmembrane domain, and an extracellular domain; wherein the intracellular domain comprises an “N-terminus domain” (ND) and an “effector domain” (ED), wherein the ND and/or the ED are associated with the luminal surface of the EV, e.g., an exosome. As used herein the term “associated with” refers to the interaction between a scaffold protein with the luminal surface of the EV, e.g., and exosome, that does not involve covalent linking to a membrane component. For example, the scaffolds useful for the present disclosure can be associated with the luminal surface of the EV, e.g., via a lipid anchor (e.g., myristic acid), and/or a polybasic domain that interacts electrostatically with the negatively charged head of membrane phospholipids. In other aspects, the Scaffold Y protein comprises an N-terminus domain (ND) and an effector domain (ED), wherein the ND is associated with the luminal surface of the EV and the ED are associated with the luminal surface of the EV by an ionic interaction, wherein the ED comprises at least two, at least three, at least four, at least five, at least six, or at least seven contiguous basic amino acids, e.g., lysines (Lys), in sequence.

In other aspects, the Scaffold Y protein comprises an N-terminus domain (ND) and an effector domain (ED), wherein the ND is associated with the luminal surface of the EV, e.g., exosome, and the ED is associated with the luminal surface of the EV by an ionic interaction, wherein the ED comprises at least two, at least three, at least four, at least five, at least six, or at least seven contiguous basic amino acids, e.g., lysines (Lys), in sequence.

In some aspects, the ND is associated with the luminal surface of the EV, e.g., an exosome, via lipidation, e.g., via myristoylation. In some aspects, the ND has Gly at the N terminus. In some aspects, the N-terminal Gly is myristoylated.

In some aspects, the ED is associated with the luminal surface of the EV, e.g., an exosome, by an ionic interaction. In some aspects, the ED is associated with the luminal surface of the EV, e.g., an exosome, by an electrostatic interaction, in particular, an attractive electrostatic interaction.

In some aspects, the ED comprises (i) a basic amino acid (e.g., lysine), or (ii) two or more basic amino acids (e.g., lysine) next to each other in a polypeptide sequence. In some aspects, the basic amino acid is lysine (Lys; K), arginine (Arg, R), or Histidine (His, H). In some aspects, the basic amino acid is (Lys)n, wherein n is an integer between 1 and 10.

In other aspects, the ED comprises at least a lysine and the ND comprises a lysine at the C terminus if the N terminus of the ED is directly linked to lysine at the C terminus of the ND, i.e., the lysine is in the N terminus of the ED and is fused to the lysine in the C terminus of the ND. In other aspects, the ED comprises at least two lysines, at least three lysines, at least four lysines, at least five lysines, at least six lysines, or at least seven lysines when the N terminus of the ED is linked to the C terminus of the ND by a linker, e.g., one or more amino acids.

Non-limiting examples of the Scaffold Y protein useful for the present disclosure are disclosed in International Publication No. WO/2019/099942, which is incorporated by reference herein in its entirety.

In some aspects, the Scaffold Y protein useful for the present disclosure does not contain an N-terminal Met. In some aspects, the Scaffold Y protein comprises a lipidated amino acid, e.g., a myristoylated amino acid, at the N-terminus of the scaffold protein, which functions as a lipid anchor. In some aspects, the amino acid residue at the N-terminus of the scaffold protein is Gly. The presence of an N-terminal Gly is an absolute requirement for N-myristoylation. In some aspects, the amino acid residue at the N-terminus of the scaffold protein is synthetic. In some aspects, the amino acid residue at the N-terminus of the scaffold protein is a glycine analog, e.g., allylglycine, butylglycine, or propargylglycine.

In other aspects, the lipid anchor can be any lipid anchor known in the art, e.g., palmitic acid or glycosylphosphatidylinositols. Under unusual circumstances, e.g., by using a culture medium where myristic acid is limiting, some other fatty acids including shorter-chain and unsaturated, can be attached to the N-terminal glycine. For example, in BK channels, myristate has been reported to be attached posttranslationally to internal serine/threonine or tyrosine residues via a hydroxyester linkage. Membrane anchors known in the art are presented in the following table:

| Modification | Modifying Group |
|---|---|
| S-Palmitoylation | 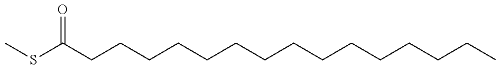 [SMILES: CCCCCCCCCCCCCCCC(=O)SC] |
| N-Palmitoylation | 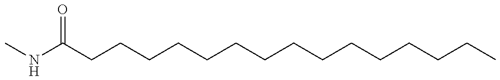 [SMILES: CCCCCCCCCCCCCCCC(=O)NC] |
| N-Myristoylation | 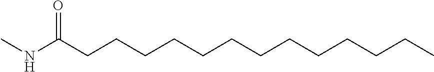 [SMILES: CCCCCCCCCCCCCC(=O)NC] |
| O-Acylation | 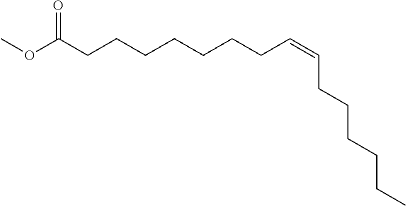 [SMILES: CCCCCC/C=C\CCCCCCCC(=O)OC] |
| Farnesylation | 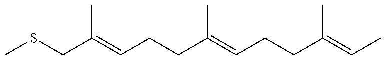 [SMILES: C/C=C(\C)CC/C=C(\C)CC/C=C(\C)CSC] |
| Geranylgeranylation | 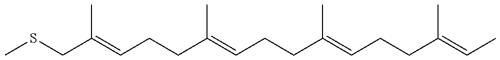 [SMILES: C/C=C(\C)CC/C=C(\C)CC/C=C(\C)CC/C=C(\C)CSC] |
| Cholesterol | 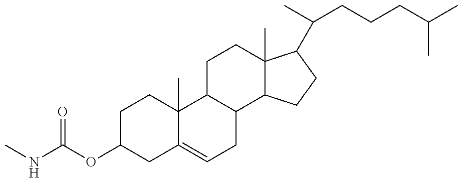 [SMILES: CNC(=O)OC1CCC2(C)C(=CCC3C2CCC2(C)C(C(C)CCCC(C)C)CCC32)C1] |

### III.B. Linkers

As described supra, extracellular vesicles (EVs) of the present disclosure (e.g., exosomes) can comprises one or more linkers that link a molecule of interest (e.g., a payload, e.g., an IL-12 moiety) to the EVs (e.g., to the exterior surface or on the luminal surface). In some aspects, a payload, e.g., biologically active molecule, is linked to the EVs directly or via a scaffold moiety (e.g., Scaffold X or Scaffold Y). In certain aspects, the payload, e.g., biologically active molecule, is linked to the scaffold moiety by a linker. In certain aspects, the payload, e.g., biologically active molecule, is linked to the second scaffold moiety by a linker.

In certain aspects, the payload, e.g., biologically active molecule, is linked to the exterior surface of an exosome via Scaffold X. In further aspects, the payload, e.g., biologically active molecule, is linked to the luminal surface of an exosome via Scaffold X or Scaffold Y. The linker can be any chemical moiety known in the art.

In some aspects, two or more linkers can be linked in tandem. When multiple linkers are present, each of the linkers can be the same or different. Generally, linkers provide flexibility or prevent/ameliorate steric hindrances. Linkers are not typically cleaved; however, in certain aspects, such cleavage can be desirable. Accordingly, in some aspects, a linker can comprise one or more protease-cleavable sites, which can be located within the sequence of the linker or flanking the linker at either end of the linker sequence.

In some aspects, the linker is a peptide linker. In some aspects, the peptide linker can comprise at least about two, at least about three, at least about four, at least about five, at least about 10, at least about 15, at least about 20, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, or at least about 100 amino acids.

In some aspects, the peptide linker is synthetic, i.e., non-naturally occurring. In one aspect, a peptide linker includes peptides (or polypeptides) (e.g., natural or non-naturally occurring peptides) which comprise an amino acid sequence that links or genetically fuses a first linear sequence of amino acids to a second linear sequence of amino acids to which it is not naturally linked or genetically fused in nature. For example, in one aspect the peptide linker can comprise non-naturally occurring polypeptides which are modified forms of naturally occurring polypeptides (e.g., comprising a mutation such as an addition, substitution or deletion).

In some aspects, the linker is a non-polypeptide moiety.

Linkers can be susceptible to cleavage (“cleavable linker”) thereby facilitating release of the biologically active molecule (e.g., an IL-12 moiety).

In some aspects, the linker is a “reduction-sensitive linker.” In some aspects, the reduction-sensitive linker contains a disulfide bond. In some aspects, the linker is an “acid labile linker.” In some aspects, the acid labile linker contains hydrazone. Suitable acid labile linkers also include, for example, a cis-aconitic linker, a hydrazide linker, a thiocarbamoyl linker, or any combination thereof.

In some aspects, the linker comprises a non-cleavable linker.

### III.C. Therapeutic Agents

In some aspects, the EVs described herein can further comprise at least one therapeutic agent or a biologically active molecule. In some aspects, the therapeutic agent comprises a cytokine (e.g., an IL-12), a small molecule, a growth factor, an antigen, an antisense oligonucleotide, an siRNA, an shRNA, a miRNA, a dsDNA, a lncRNA, a PROTAC, an adjuvant, an immune modulator, or any combination thereof.

In some aspects, the antisense oligonucleotide modulates the function of a target gene. In some aspects, the ASO modulates the function of nucleic acid molecules encoding mammalian STAT6 (e.g., described in WO2021030776, herein incorporated by reference), such as the STAT6 nucleic acid, e.g., STAT6 transcript, including STAT6 pre-mRNA, and STAT6 mRNA, or naturally occurring variants of such nucleic acid molecules encoding mammalian STAT6. In some aspects, the ASO modulates the function of nucleic acid molecules encoding mammalian STAT3 (e.g., described in WO2021030768, which is herein incorporated by reference in its entirety) such as the STAT3 nucleic acid, e.g., STAT3 transcript, including STAT3 pre-mRNA, and STAT3 mRNA, or naturally occurring variants of such nucleic acid molecules encoding mammalian STAT3. In some aspects, the ASO modulates the function of nucleic acid molecules encoding mammalian CEBP/b (e.g., described in WO2021030780, which is herein incorporated by reference in its entirety), such as the CEBP b nucleic acid, e.g., CEBP b transcript, including CEBP b pre-mRNA, and CEBP b mRNA, or naturally occurring variants of such nucleic acid molecules encoding mammalian CEBP/b. In some aspects, the ASO modulates the function of nucleic acid molecules encoding mammalian NRAS (e.g., described in WO2021030769, which is herein incorporated by reference in its entirety), such as the NRAS nucleic acid, e.g., NRAS transcript, including NRAS pre-mRNA, and NRAS mRNA, or naturally occurring variants of such nucleic acid molecules encoding mammalian NRAS. In some aspects, the ASO modulates the function of nucleic acid molecules encoding mammalian KRAS (e.g., described in WO2021030781, which is herein incorporated by reference in its entirety), such as the KRAS nucleic acid, e.g., KRAS transcript, including KRAS pre-mRNA, and KRAS mRNA, or naturally occurring variants of such nucleic acid molecules encoding mammalian KRAS. The term “ASO” in the context of the present disclosure, refers to a molecule formed by covalent linkage of two or more nucleotides (i.e., an oligonucleotide).

In some aspects, the therapeutic agent comprises a STING agonist, as described in WO 2019/183578.

In some aspects, the present disclosure provides methods of treating or preventing a disease or a condition in a subject in need thereof comprising administering the extracellular vesicles described herein. The present disclosure also provides use of the extracellular vesicles described herein to treat or prevent a disease or condition in a subject in need thereof. In some aspects, the present disclosure provides the extracellular vesicles described herein for treating or preventing a disease or a condition in a subject in need thereof.

### IV. Producer Cells

EVs, e.g., exosomes, of the present disclosure can be produced from a cell grown in vitro or a body fluid of a subject. When exosomes are produced from in vitro cell culture, various producer cells, e.g., HEK293 cells, CHO cells, C2C12, and MSCs, can be used. In some aspects, the producer cells can be selected from HEK293 cells, HEK293S cells, HEK293SF cells, Chinese Hamster Ovary (CHO) cells, mesenchymal stem cells (MSCs), BJ human foreskin fibroblast cells, fHIDF fibroblast cells, AGE.HN® neuronal precursor cells, CAP® amniocyte cells, adipose mesenchymal stem cells, RPTEC/TERT1 cells, dendritic cells, macrophages, B cells, mast cells, neutrophils, Kupffer-Browicz cells, PER.C6 cells, Induced pluripotent stem cells (iPSCs), or C2C12 cells. In some aspects, the producer cells are stem cells.

The producer cells can be genetically and/or pharmacologically modified to reduce gene and/or protein function in a cholesterol biosynthetic pathway, as described herein. In some aspects, the modified producer cells can be further modified (e.g., genetically) to comprise exogenous sequences encoding a protein to produce EVs described herein. The genetically-modified producer cell can contain the exogenous sequence by transient or stable transformation. The exogenous sequence can be transformed as a plasmid. In some aspects, the exogenous sequence is a vector. The exogenous sequences can be stably integrated into a genomic sequence of the producer cell, at a targeted site or in a random site. In some aspects, a stable cell line is generated for production of lumen-engineered exosomes.

The exogenous sequences can be inserted into a genomic sequence of the producer cell, located within, upstream (5′-end) or downstream (3′-end) of an endogenous sequence encoding an exosome protein. Various methods known in the art can be used for the introduction of the exogenous sequences into the producer cell. For example, cells modified using various gene editing methods (e.g., methods using a homologous recombination, transposon-mediated system, loxP-Cre system, CRISPR/Cas9 or TALEN) are within the scope of the present disclosure.

The exogenous sequences can comprise a sequence encoding a scaffold moiety disclosed herein or a fragment or variant thereof. An extra copy of the sequence encoding a scaffold moiety can be introduced to produce an exosome described herein (e.g., having a higher density of a scaffold moiety on the surface or on the luminal surface of the EV, e.g., exosome). An exogenous sequence encoding a modification or a fragment of a scaffold moiety can be introduced to produce a lumen-engineered and/or surface-engineered exosome containing the modification or the fragment of the scaffold moiety.

In some aspects, a producer cell can be modified, e.g., transfected, with one or more vectors encoding a scaffold moiety linked to a protein.

In some aspects, a producer cell disclosed herein is further modified to comprise an additional exogenous sequence. For example, an additional exogenous sequence can be introduced to modulate endogenous gene expression, or produce an exosome including a certain polypeptide as a payload (e.g., an IL-12 moiety). In some aspects, the producer cell is modified to comprise two exogenous sequences, one encoding a scaffold moiety (e.g., Scaffold X and/or Scaffold Y), or a variant or a fragment thereof, and the other encoding a payload). In some aspects, the producer cell is modified to comprise two exogenous sequences, one encoding a scaffold moiety disclosed herein, or a variant or a fragment thereof, and the other encoding a protein conferring the additional functionalities to exosomes. In some aspects, the producer cell is further modified to comprise one, two, three, four, five, six, seven, eight, nine, or ten or more additional exogenous sequences.

In some aspects, EVs, e.g., exosomes, of the present disclosure (e.g., surface-engineered and/or lumen-engineered exosomes) can be produced from a cell transformed with a sequence encoding a full-length, mature scaffold moiety disclosed herein or a scaffold moiety linked to a protein, e.g., a therapeutic protein. Any of the scaffold moieties described herein can be expressed from a plasmid, an exogenous sequence inserted into the genome or other exogenous nucleic acid, such as a synthetic messenger RNA (mRNA).

In some aspects, the present disclosure provides a bioreactor comprising extracellular vesicle (EV) producer cells, which exhibit a reduced gene and/or protein function in a cholesterol biosynthetic pathway. In some aspects, the methods described herein are performed using a perfusion bioreactor. In some aspects, the bioreactor is connected to a cell retention device.

In some aspects, the producer cells can be cultured in a bioreactor (e.g., a WAVE bioreactor, a stirred-tank bioreactor, a shaken bioreactor). Various configurations of bioreactors are known in the art and a suitable configuration can be chosen as desired. In some aspects, the culture is performed in, e.g., an N−1 or N-terminal vessel, or a bioreactor. In some aspects, the culture vessel is a fed-batch or perfusion bioreactor. In some aspects, a bioreactor is connected to a cell separator. For example, an N-terminal production vessel, in particular, a stirred-tank bioreactor, connected to a cell retention device, such as hollow fibrous membranes (run in, e.g., alternating tangential flow filtration (ATF), tangential flow filtration (TFF)), or an acoustic cell separator can be used.

### IV. Cell Compositions

In some aspects, the present disclosure provides a cell composition comprising modified cells that produce extracellular vesicles (EV), wherein the modified cells have a reduced gene and/or protein function in a cholesterol biosynthetic pathway. In some aspects, the reduced gene and/or protein comprises protein in a cholesterol biosynthesis comprises one or more genes selected from 3-hydroxy-3-methylglutaryl-coenzyme A reductase (HMGCR), sterol regulatory element-binding protein 2 (SREBF2), Squalene epoxidase (SQLE), or 7-Dehydrocholesterol reductase (DHCR7) or a protein encoded by the gene.

In some aspects, the present disclosure provides producer cells for use in the methods described herein. In some aspects, the producer cells are prepared according to the methods described herein.

### V. Pharmaceutical Compositions and Methods of Treatment

Provided herein are pharmaceutical compositions comprising an EV, e.g., exosome, of the present disclosure having the desired degree of purity, and a pharmaceutically acceptable carrier or excipient, in a form suitable for administration to a subject. Pharmaceutically acceptable excipients or carriers can be determined in part by the particular composition being administered, as well as by the particular method used to administer the composition. Accordingly, there is a wide variety of suitable formulations of pharmaceutical compositions comprising a plurality of extracellular vesicles. (See, e.g., Remington's Pharmaceutical Sciences, Mack Publishing Co., Easton, Pa. 21st ed. (2005)). The pharmaceutical compositions are generally formulated sterile and in full compliance with all Good Manufacturing Practice (GMP) regulations of the U.S. Food and Drug Administration.

In some aspects, a pharmaceutical composition comprises one or more therapeutic agents and an exosome described herein. In certain aspects, the EVs, e.g., exosomes, are co-administered with one or more additional therapeutic agents in a pharmaceutically acceptable carrier. In some aspects, the pharmaceutical composition comprising the EV, e.g., exosome is administered prior to administration of the additional therapeutic agent. In other aspects, the pharmaceutical composition comprising the EV, e.g., exosome, is administered after the administration of the additional therapeutic agent. In further aspects, the pharmaceutical composition comprising the EV, e.g., exosome, is administered concurrently with the additional therapeutic agent.

Acceptable carriers, excipients, or stabilizers are nontoxic to recipients (e.g., animals or humans) at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Examples of carriers or diluents include, but are not limited to, water, saline, Ringer's solutions, dextrose solution, and 5% human serum albumin. The use of such media and compounds for pharmaceutically active substances is well known in the art. Except insofar as any conventional media or compound is incompatible with the extracellular vesicles described herein, use thereof in the compositions is contemplated. Supplementary therapeutic agents can also be incorporated into the compositions. Typically, a pharmaceutical composition is formulated to be compatible with its intended route of administration. The EVs, e.g., exosomes, can be administered by parenteral, topical, intravenous, oral, subcutaneous, intra-arterial, intradermal, transdermal, rectal, intracranial, intraperitoneal, intranasal, intratumoral, intramuscular route or as inhalants. In certain aspects, the pharmaceutical composition comprising exosomes is administered intravenously, e.g. by injection. The EVs, e.g., exosomes, can optionally be administered in combination with other therapeutic agents that are at least partly effective in treating the disease, disorder or condition for which the EVs, e.g., exosomes, are intended.

Solutions or suspensions can include the following components: a sterile diluent such as water, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The preparation can be enclosed in ampoules, disposable syringes or multiple dose vials made of glass or plastic.

Pharmaceutical compositions suitable for injectable use include sterile aqueous solutions (if water soluble) or dispersions and sterile powders. For intravenous administration, suitable carriers include physiological saline, bacteriostatic water, Cremophor EL™ (BASF, Parsippany, N.J.) or phosphate buffered saline (PBS). The composition is generally sterile and fluid to the extent that easy syringeability exists. The carrier can be a solvent or dispersion medium containing, e.g., water, ethanol, polyol (e.g., glycerol, propylene glycol, and liquid polyethylene glycol, and the like), and suitable mixtures thereof. The proper fluidity can be maintained, e.g., by the use of a coating such as lecithin, by the maintenance of the required particle size in the case of dispersion and by the use of surfactants. Prevention of the action of microorganisms can be achieved by various antibacterial and antifungal compounds, e.g., parabens, chlorobutanol, phenol, ascorbic acid, thimerosal, and the like. If desired, isotonic compounds, e.g., sugars, polyalcohols such as manitol, sorbitol, and sodium chloride can be added to the composition. Prolonged absorption of the injectable compositions can be brought about by including in the composition a compound which delays absorption, e.g., aluminum monostearate and gelatin.

Sterile injectable solutions can be prepared by incorporating the EVs, e.g., exosomes, in an effective amount and in an appropriate solvent with one or more ingredients enumerated herein or known in the art, as desired. Generally, dispersions are prepared by incorporating the EVs, e.g., exosomes, into a sterile vehicle that contains a basic dispersion medium and any desired other ingredients. In the case of sterile powders for the preparation of sterile injectable solutions, methods of preparation are vacuum drying and freeze-drying that yield a powder of the active ingredient plus any additional desired ingredient from a previously sterile-filtered solution thereof. The EVs, e.g., exosomes, can be administered in the form of a depot injection or implant preparation which can be formulated in such a manner to permit a sustained or pulsatile release of the EV, e.g., exosome.

Systemic administration of compositions comprising exosomes can also be by transmucosal means. For transmucosal administration, penetrants appropriate to the barrier to be permeated are used in the formulation. Such penetrants are generally known in the art, and include, e.g., for transmucosal administration, detergents, bile salts, and fusidic acid derivatives. Transmucosal administration can be accomplished through the use of, e.g., nasal sprays.

In certain aspects the pharmaceutical composition comprising EVs, e.g., exosomes is administered intravenously into a subject that would benefit from the pharmaceutical composition. In certain other aspects, the composition is administered to the lymphatic system, e.g., by intralymphatic injection or by intranodal injection (see e.g., Senti et al., PNAS 105(46): 17908 (2008)), or by intramuscular injection, by subcutaneous administration, by intratumoral injection, by direct injection into the thymus, or into the liver.

In certain aspects, the pharmaceutical composition comprising exosomes is administered as a liquid suspension. In certain aspects, the pharmaceutical composition is administered as a formulation that is capable of forming a depot following administration. In certain preferred aspects, the depot slowly releases the EVs, e.g., exosomes, into circulation, or remains in depot form.

Typically, pharmaceutically-acceptable compositions are highly purified to be free of contaminants, are biocompatible and not toxic, and are suited to administration to a subject. If water is a constituent of the carrier, the water is highly purified and processed to be free of contaminants, e.g., endotoxins.

The pharmaceutically-acceptable carrier can be lactose, dextrose, sucrose, sorbitol, mannitol, starch, gum acacia, calcium phosphate, alginates, gelatin, calcium silicate, micro-crystalline cellulose, polyvinylpyrrolidone, cellulose, water, syrup, methyl cellulose, methylhydroxy benzoate, propylhydroxy benzoate, talc, magnesium stearate, and/or mineral oil, but is not limited thereto. The pharmaceutical composition can further include a lubricant, a wetting agent, a sweetener, a flavor enhancer, an emulsifying agent, a suspension agent, and/or a preservative.

The pharmaceutical compositions described herein comprise the EVs, e.g., exosomes, described herein and optionally a pharmaceutically active or therapeutic agent. The therapeutic agent can be a biological agent, a small molecule agent, or a nucleic acid agent.

Dosage forms are provided that comprise a pharmaceutical composition comprising the EVs, e.g., exosomes, described herein. In some aspects, the dosage form is formulated as a liquid suspension for intravenous injection. In some aspects, the dosage form is formulated as a liquid suspension for intratumoral injection.

In certain aspects, the preparation of exosomes is subjected to radiation, e.g., X rays, gamma rays, beta particles, alpha particles, neutrons, protons, elemental nuclei, UV rays in order to damage residual replication-competent nucleic acids.

In certain aspects, the preparation of exosomes is subjected to gamma irradiation using an irradiation dose of more than 1, 5, 10, 15, 20, 25, 30, 35, 40, 50, 60, 70, 80, 90, 100, or more than 100 kGy.

In certain aspects, the preparation of exosomes is subjected to X-ray irradiation using an irradiation dose of more than 0.1, 0.5, 1, 5, 10, 15, 20, 25, 30, 35, 40, 50, 60, 70, 80, 90, 100, 200, 300, 400, 500, 600, 700, 800, 900, 1000, 2000, 3000, 4000, 5000, 6000, 7000, 8000, 9000, 10000, or greater than 10000 mSv.

The present disclosure also provides methods of treating a disease or disorder in a subject in need thereof comprising administering the extracellular vesicle described herein to the subject.

### EXAMPLES

### Example 1

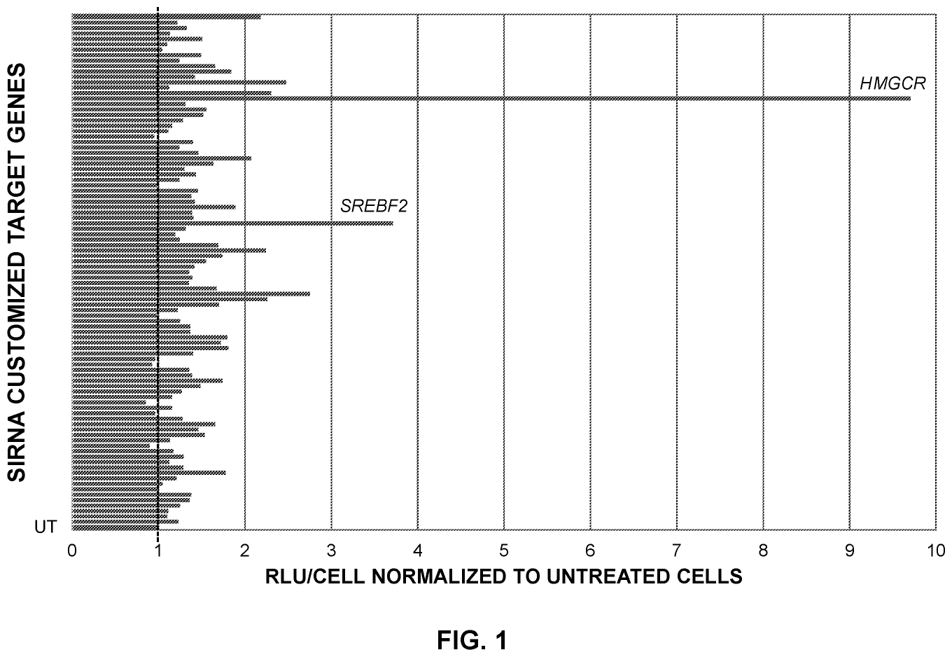

To determine if EV production from producer cells could be improved, a high-throughput EV reporter assay was developed. The reporter assay has a large dynamic range (e.g., 10⁵ to 10¹¹) capable of measuring EV associated luminescence in minimally processed samples. The EV producer cells express a PTGRN-FLAG-HiBiT luciferase protein, providing a fluorescent signal associated with the produced EVs. siRNA reagents were optimized for use in the HEK HiBit reporter cells. A siRNA knock down screen of approximately 200 genes was performed and HiBit luciferase was assessed as a measure of EV production. Knock down of HMGCR and SREBF2 were identified as significantly increasing EV production compared to untreated control cells (FIG. 1).

### Example 2

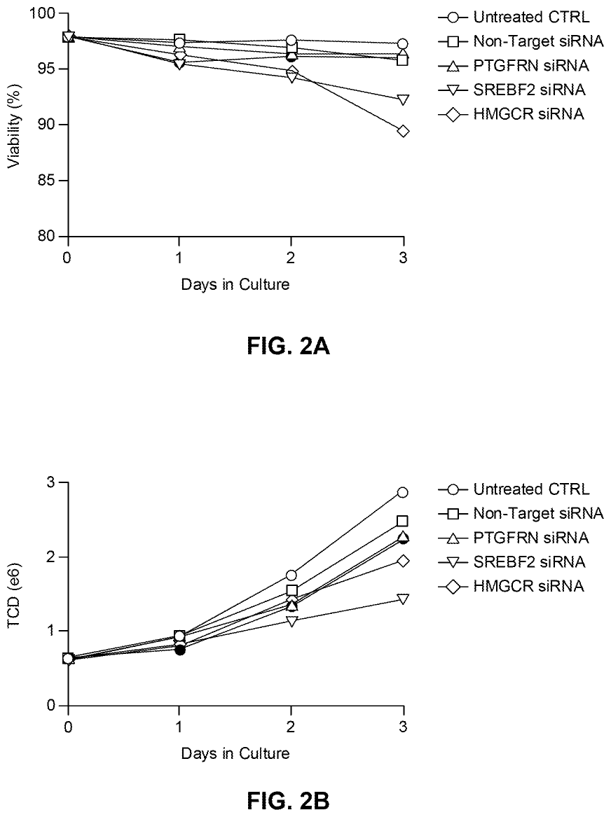

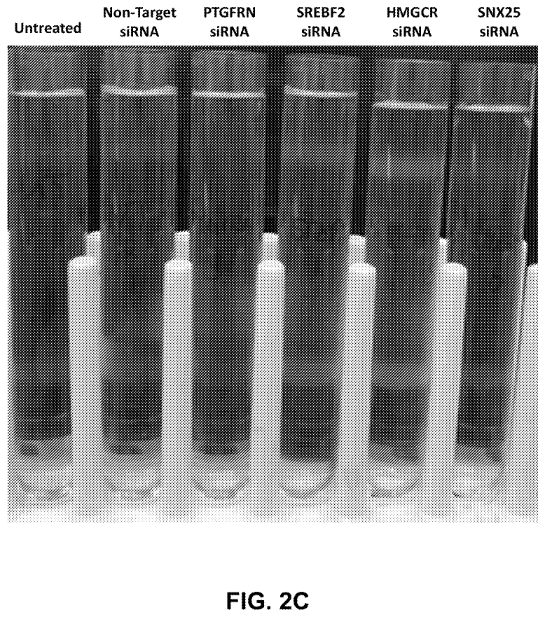

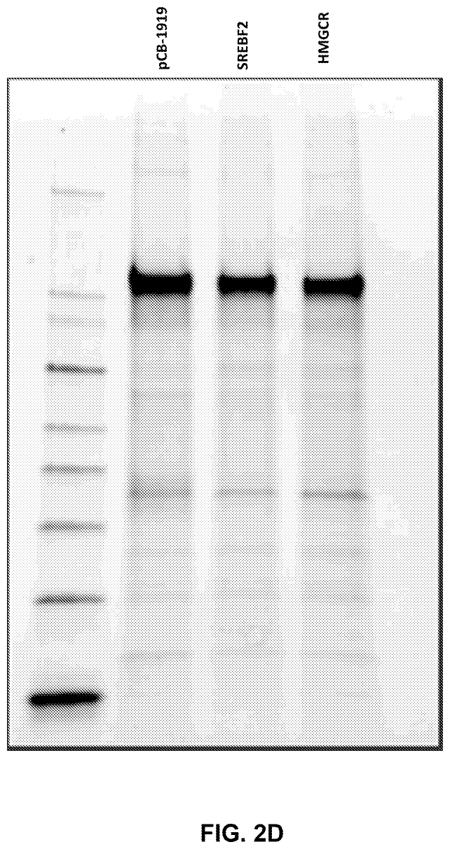

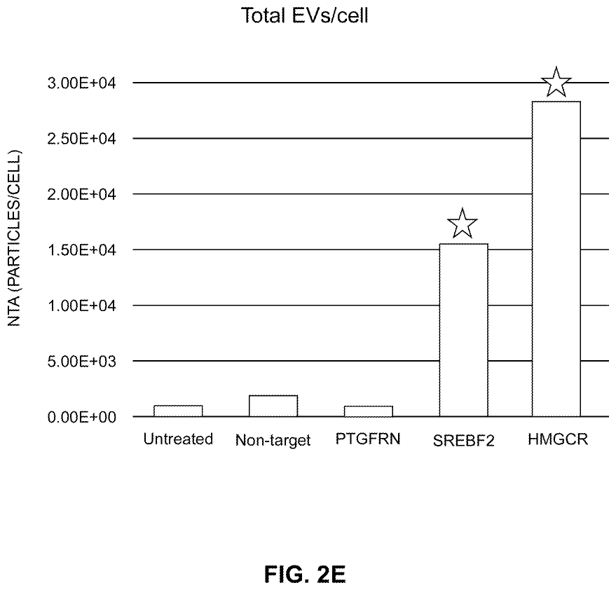

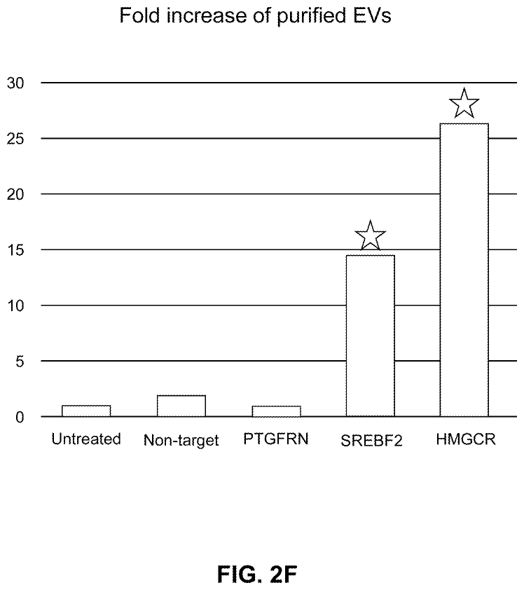

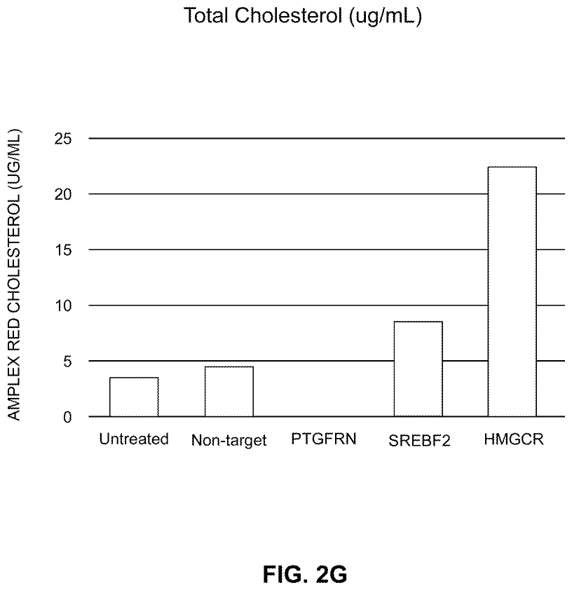

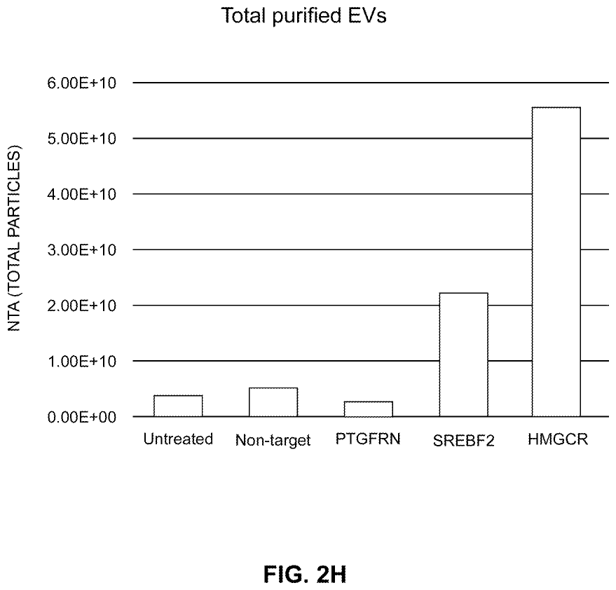

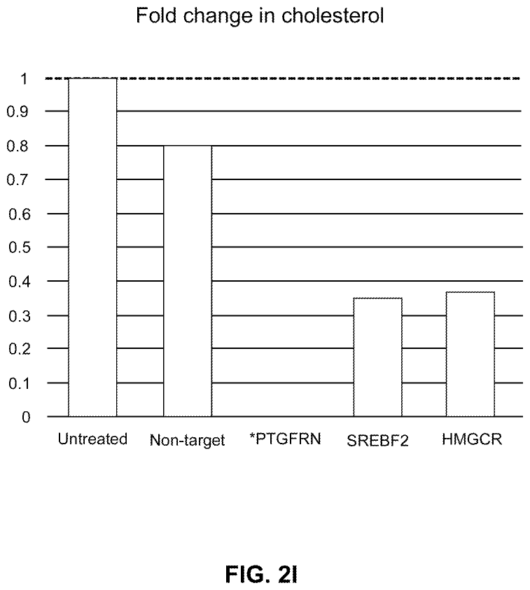

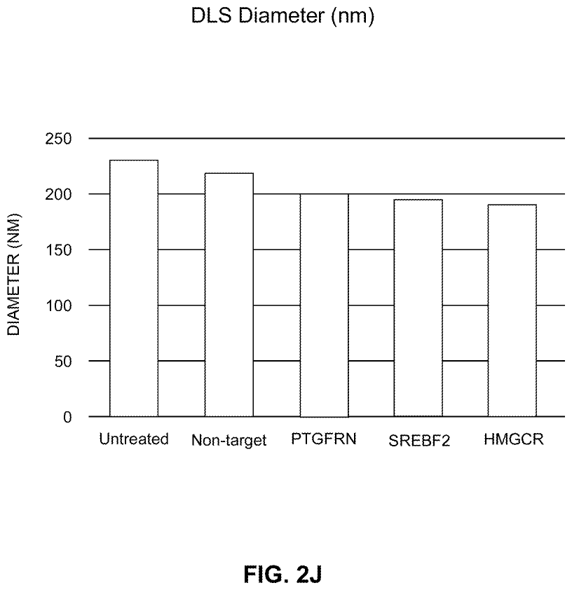

In order to validate that knock down of HMGCR and SREBF2 in producer cells increased EV production, nanoparticle tracking analysis (NTA) was performed from samples collected from untreated producer cells, non-target siRNA treated producer cells, PTGFRN knock down producer cells, SREBF2 knock down producer cells, and HMGCR knock down producer cells. The viable cell density and viability (%) for untreated producer cells, non-target siRNA treated producer cells, PTGFRN knock down producer cells, SREBF2 knock down producer cells, and HMGCR knock down producer cells was determined (FIGS. 2A and 2B) and EVs were purified and characterized (FIGS. 2C and 2D). Total EVs produced per cell was significantly increased in SREBF2 knock down producer cells and HMGCR knock down producer cells (FIG. 2E). SREBF2 knock down resulted in approximately 15 fold increase in EV production compared to untreated producer cells and HMGCR knock down resulted in approximately 25 fold increase compared to untreated producer cells (FIG. 2F). Total cholesterol and total purified EVs were quantitated for each treatment group (FIGS. 2G and 2H, respectively). Fold change in cholesterol normalized to total EVs was calculated showing the difference in cholesterol content of the EVs for the treatment groups (FIG. 2I). No major difference in size distribution from the EVs purified from the untreated producer cells, non-target siRNA treated producer cells, PTGFRN knock down producer cells, SREBF2 knock down producer cells, and HMGCR knock down producer cells was observed (FIG. 2J).

### Example 3

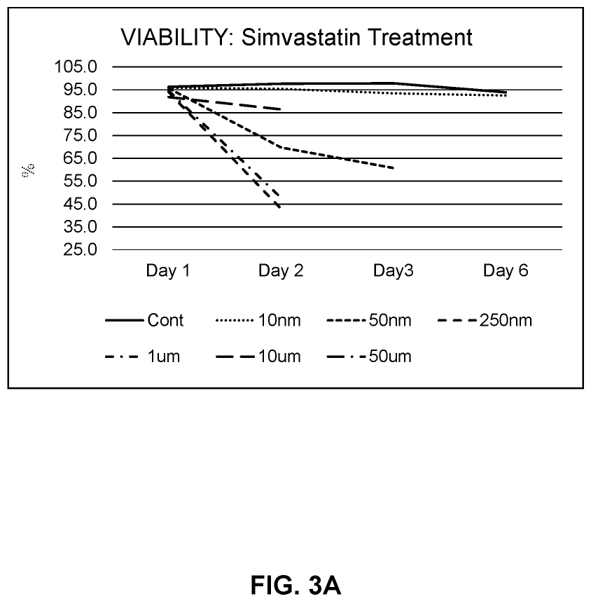

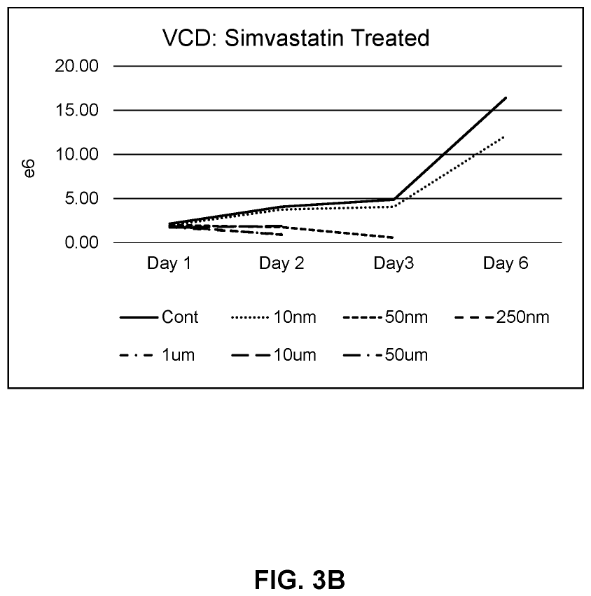

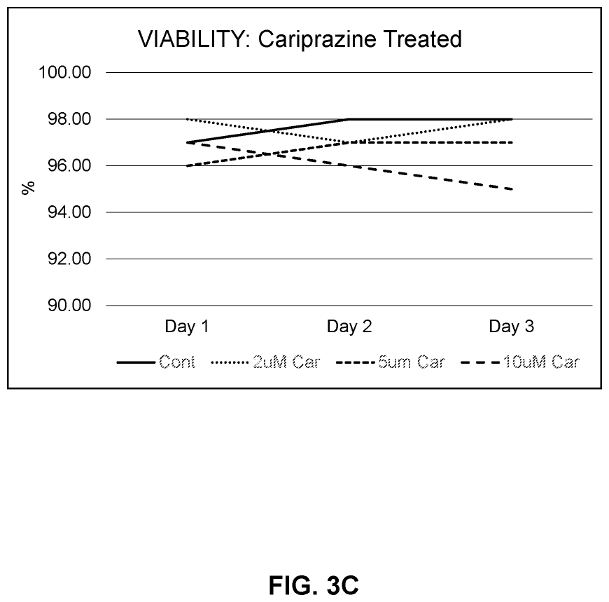

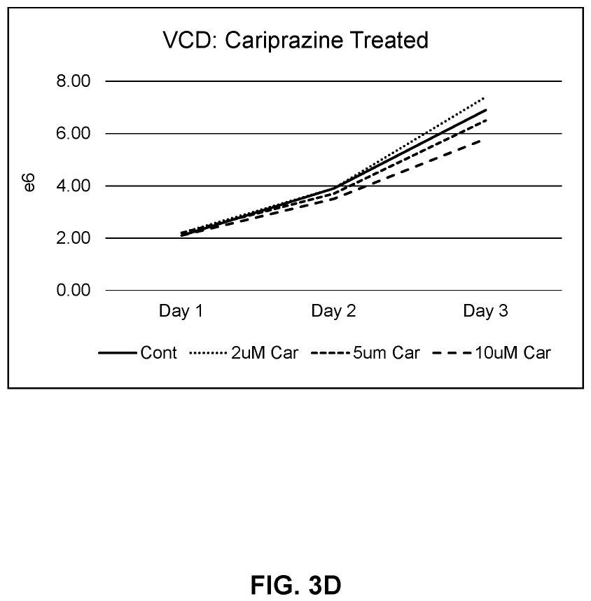

These results were further validated and confirmed by supplementing the media used for culturing producer cells with simvastatin and cariprazine. The effect of simvastatin and cariprazine treatment of producer cell viability and viable cell density was evaluated in the HiBit reporter cell line (FIGS. 3A, 3B, 3C, 3D, respectively). The effect of simvastatin and cariprazine treatments on HiBit luciferase relative to control is shown in Tables 5 and 6, respectively.

TABLE 5
Luciferase relative to control for simvastatin treatment.

| | Day 2 | Day 3 | Day 6 |
|---|---|---|---|
| Control | 1 | 1 | 1 |
| 10 nM Simvastatin | 2.3 | 4.7 | 9.6 |
| 50 nM Simvastatin | 5.3 | | |

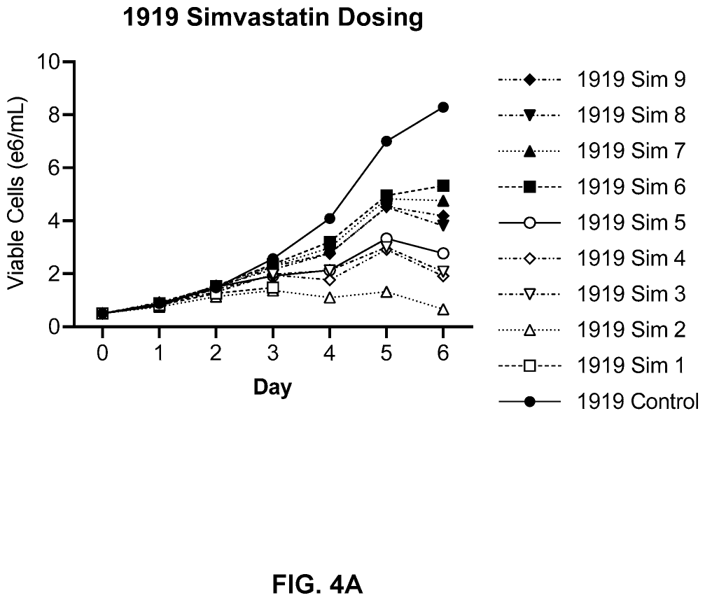

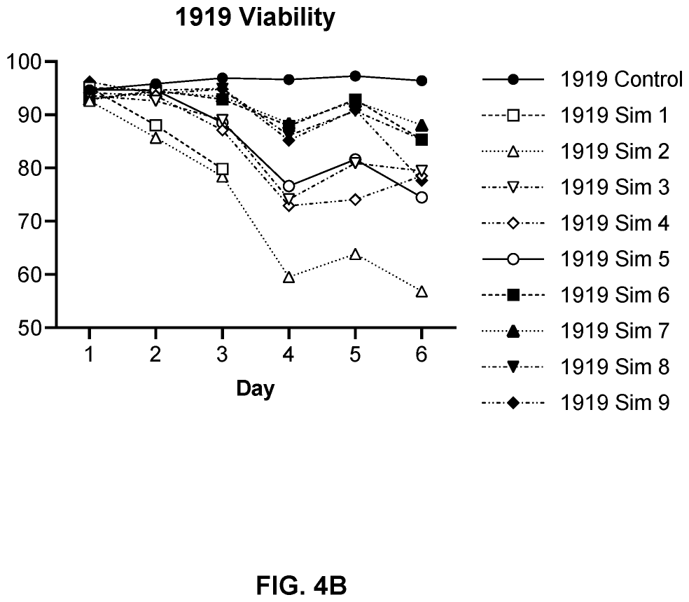

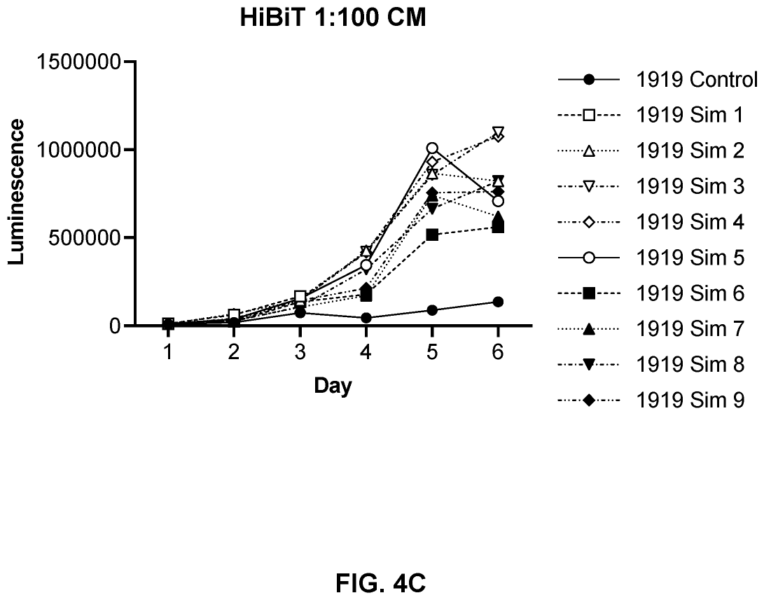

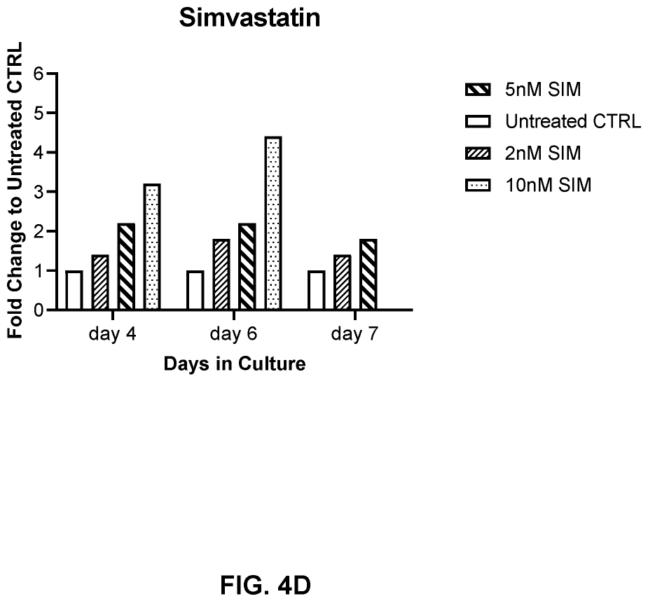

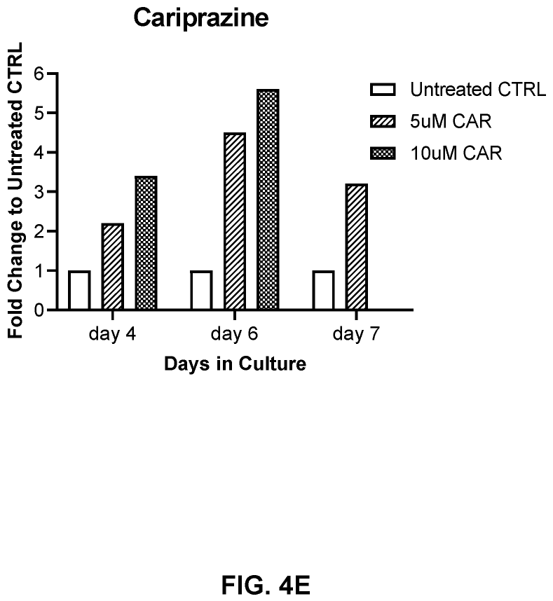

Batch refeed in shake flask with 1919 HiBit reporter cell line was performed. Cells were refed on Day 2 and 3. The 10 nM dose was well tolerated over the duration of the study. FIGS. 4A, 4B, and 4C show the effect of simvastatin dosing on viable cell density, viability, and the HiBit luciferase reporter (FIGS. 4A, 41B, 4C, respectively.)

TABLE 6
Luciferase relative to control for cariprazine treatment.

| | Day 2 | Day 3 |
|---|---|---|
| Control | 1 | 1 |
| 2 μM Cariprazine | 0.8 | 1.8 |
| 5 μM Cariprazine | 1 | 2.6 |
| 10 μM Cariprazine | 0.8 | 2.4 |

One media exchange on day 2 using the 1919 HiBit reporter cell line was performed. All conditions were well tolerated.

To further validate these results, EV production was assessed in the presence of 2 nM, 5 nM, 10 nM simvastatin supplemented into the media, and compared to untreated control (FIG. 4D), the results of which are quantified in Table 7 below. The IC₅₀ was determined to be between about 10 to about 20 nanomolar Simvastatin and similar increases in EV yield were obtained.

TABLE 7
Viability and Viable Cell Density (VCD)
of simvastatin treated producer cells.

| | Day 4 | | | Day 6 | | |
|---|---|---|---|---|---|---|
| Condition | Viability | Day 4 VCD | | Viability | D6 VCD | |
| Untreated | 97.5% | 9.58 | e6/ml | 96.0% | 17.56 | e6/ml |
| 2 nM | 94.9% | 10.41 | e6/ml | 95.5% | 17.00 | e6/ml |
| Simvastatin | | | | | | |
| 5 nM | 92.7% | 9.16 | e6/ml | 95.7% | 15.5 | e6/ml |
| Simvastatin | | | | | | |
| 10 nM | 88.8% | 8.66 | e6/ml | 93.6% | 12.41 | e6/ml |
| Simvastatin | | | | | | |

EV production was also assessed in the presence of 5 μM, and 10 μM Cariprazine supplemented into the media compared to untreated control (FIG. 4E), the results of which are quantified in Table 8 below.

TABLE 8
Viability and Viable Cell Density (VCD)
of cariprazine treated producer cells.

| | Day 4 | | Day 6 | | |
|---|---|---|---|---|---|
| Condition | Viability | Day 4 VCD | Viability | D6 VCD | |
| Untreated | 97.5% | 9.58 e6/ml | 96.0% | 17.56 | e6/ml |
| 5 μM | 95.6% | 7.73 e6/ml | 92.4% | 11.97 | e6/ml |
| Cariprazine | | | | | |
| 10 μM | 89.7% | 5.14 e6/ml | 81.0% | 5.13 | e6/ml |
| Cariprazine | | | | | |

These data indicate that pharmacologically modulating the cholesterol biosynthesis pathway can increase EV yield.

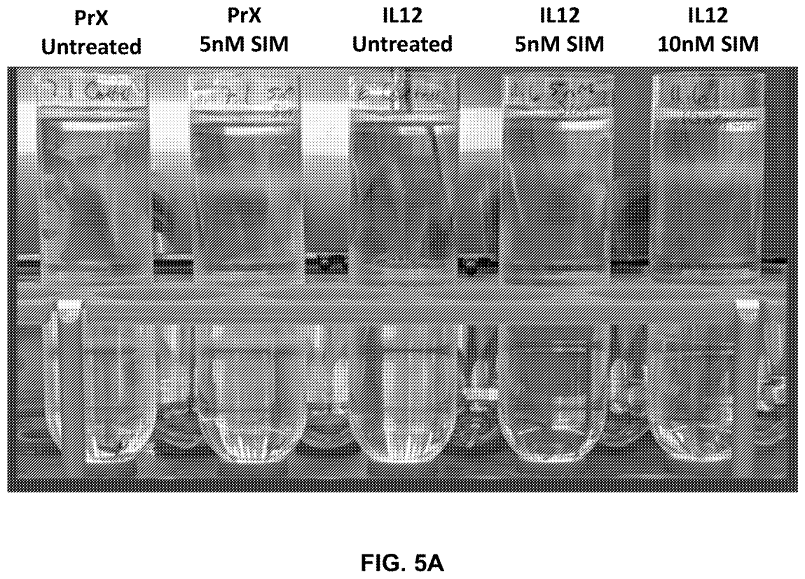

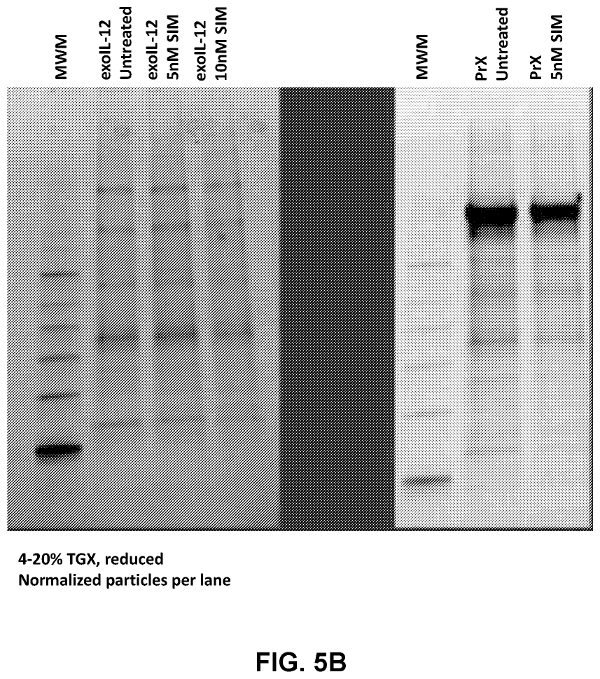

To further validate these results, the effect of simvastatin on EV yield was evaluated in two different HEK producer cell lines, one producing Protein X LVs (PrX) and the other producing IL-12 LVs (exo-12). The cells lines were treated with simvastatin (5 nM and 10 nM) for 10 days prior to EV purification. FIG. 5A shows the respective density gradient for EV purification. FIG. 5B shows a protein gel, the results of which are quantified in Table 9 below. These data indicate that Simvastatin treatment increases V yield without overt changes in V protein content.

TABLE 9
EV yield in total particles from HEK cells.

| Condition | Total Particles | Relationship to Control |
|---|---|---|
| PrX Control | 1.13E+11 | — |
| PrX 5 nM Simvastatin | 4.5E+11 | 4x |
| exoIL-12 Control | 4.96E+10 | — |
| exoIL-12 5 nM Simvastatin | 1.79E+11 | 3.5x |
| exoIL-12 10 nM Simvastatin | 3.50E+11 | 7x |

To assess if these results could be reproduced in a different cell line, CHO-S cells were treated with 10 nM simvastatin and the EV yield was compared to untreated control. Table 10 quantifies the results.

TABLE 10
EV yield in total particles from CHO cells.

| Condition | Total Particles | Relationship to Control |
|---|---|---|
| CHO-S Control | 3.93E+10 | |
| CHO-S 10 nM Simvastatin | 2.22E+11 | 5.6X |

A similar 5.6-fold increase relative to untreated controls was also observed in CHO-S cells six days after being treated with 6 nM simvastatin.

To further test whether these results could be reproduced in another cell line, human adipose MSC cells (ATCC) were grown adherent on Cellbind T125 flasks in Sartorius MSC NUTRISTEM® XF serum free media. When cultures reached approximately 50% confluence, the media was removed and replaced with the identical growth media (control) or the growth media supplemented with 6 nm simvastatin (sim treated). The cultures were incubated for an additional 4 days at which point the conditioned media was collected and the cell debris removed by centrifugation. The clarified condition media was then analyzed by anion exchange (AEX) ultra high-performance liquid chromatography (UHPLC), and the relative amount of extracellular vesicles in each sample was estimated by comparing peak areas. Table 11 quantifies the results, showing a 2.6 fold increase in EV production by adipose MSC cells treated with 6 nM simvastatin. These data are significant as MSC are a preferred cell type used in the therapeutic EV space. These results indicate that statin treatment enhancement of EV production is not unique to HEK cells.

TABLE 11
Relative EV yield from human adipose MSC cells.

| | FOLD INCREASE PER |
|---|---|
| CONDITION | CELL EV PRODUCTION |
| Adipose MSC Control | 1X |
| Adipose MSC Sim Treated | 2.6X |

### Example 4

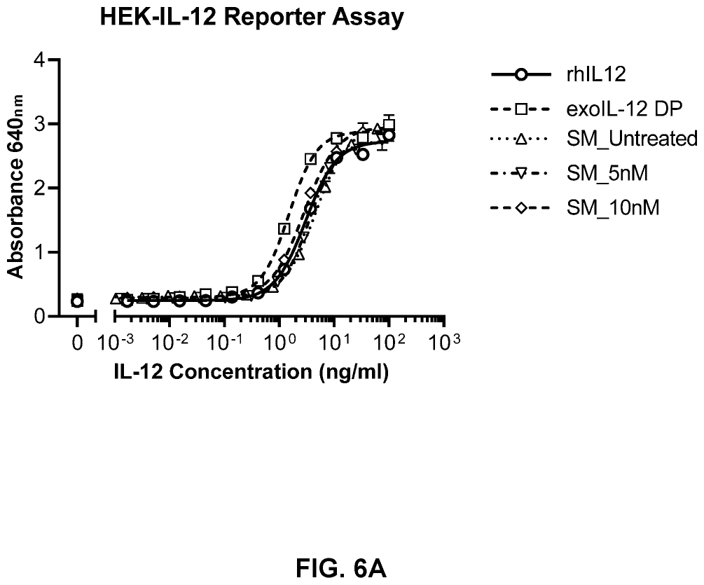

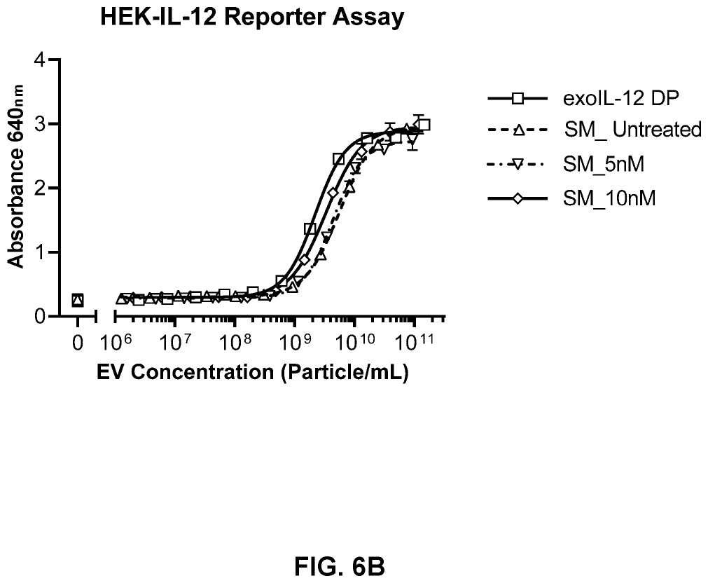

To determine if the increased EV yield from producer cells treated with a statin (e.g., simvastatin) has a functional effect on the resulting EVs, simvastatin treated exoIL-12 EVs were harvested from producer cells treated with 5 nM and 10 nM simvastatin and their potency was assessed in a functional HEK IL-12 reporter assay and compared to untreated control. FIGS. 6A and 6B show similar in vitro potency for EVs produced with simvastatin treatment compared to untreated control, recombinant human IL-12, and exoIL-12 drug product. These results demonstrate that exoIL-12 EVs purified from producer cells treated with simvastatin displayed similar in vitro potency to EVs purified from untreated producer cells.

These results were confirmed using PrX EVs purified from simvastatin treated cultures and subsequently loaded with either STING agonist or STAT6 antisense oligonucleotide. Like the exoIL-12 EVs, simvastatin treatment of exoSTING EVs and exoASO-STAT6 EVs had no effect on in vivo potency as compared to controls (data not shown).

### Example 5

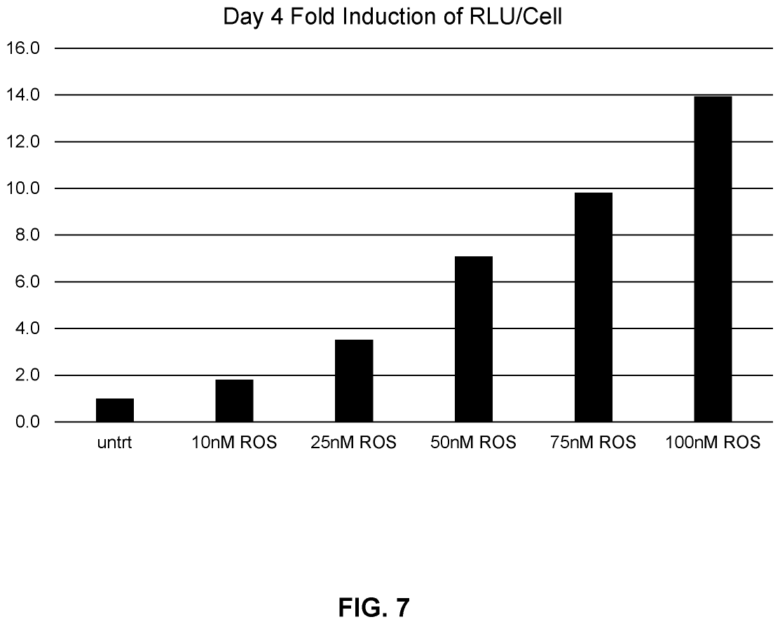

To explore if the increased EV yield from producer cells treated with a statin was generalizable to other statins, experiments were performed in which HiBit producer cells were exposed to varying concentrations of rosuvastatin (“ROS;” 10 nM, 25 nM, 50 nM, 75 nM, and 100 nM). Notably, rosuvastatin is a fully synthetic, hydrophobic molecule with additional HMGCR binding capabilities through fluorophenyl and sulfone groups. Conversely, simvastatin is a lipophilic molecule that is derived from a fungal metabolite. Yet despite these differences, rosuvastatin showed a dose-dependent increase in EV expression in treated HiBit reporter cells at four days post induction (FIG. 7). These results demonstrate that the increased EV expression observed by reducing cholesterol are not unique to simvastatin, but rather appear to be a general phenomena of statins.

### Example 6

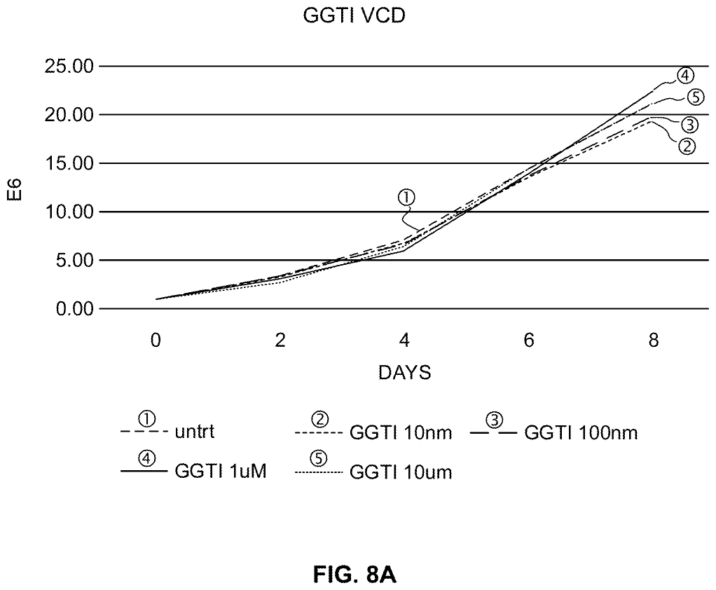

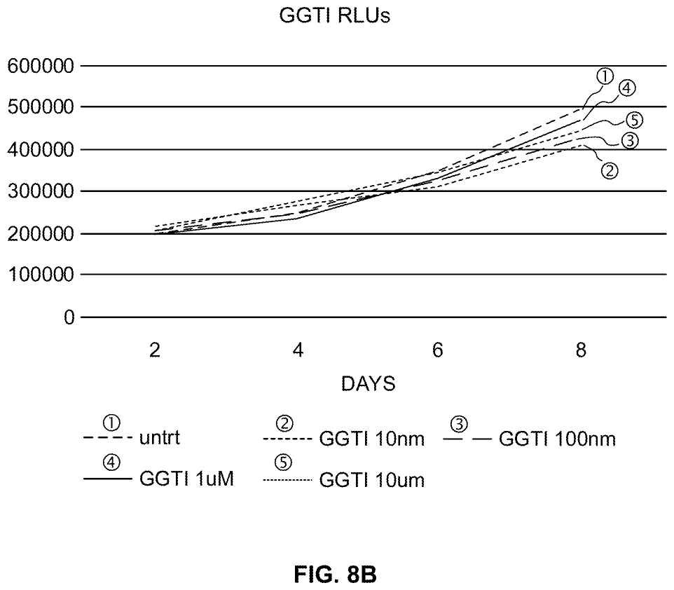

To determine whether the increase in EV expression is associated with cholesterol itself and not prenylation in general, exosome production was investigated following exposure of producer cells to a synthetic inhibitor of GGTase. Prenylation controls the activity of Rab proteins that have been shown to be involved in EV biogenesis. The precursors of prenylation are derived from cholesterol pathway intermediates that are likely to be depleted by both the statin and siRNA treatment, but which are not likely to be depleted by caraprazine treatment. Geranylgeranyltransferase (GGTase) is responsible for posttranslational modification of proteins through attachment of a prenyl residue to the carboxyl terminal of a protein. Prenylated proteins are membrane associated due to the hydrophobic nature of the prenyl group. Cultures were treated with GGTI-2418 (a peptidomimetic inhibitor of GGTase I), and viable cell density (VCD) and exosomal production (as expressed by RLUs) were evaluated at days 2, 4, 6, and 8. Exposure of cultures to GGTI-2418 had no effect on either cell density (FIG. 8A) or exosome production (FIG. 8B), suggesting that prenylation does not affect exosomal production under the culture conditions tested here, and supporting the role of cholesterol.

### Example 7

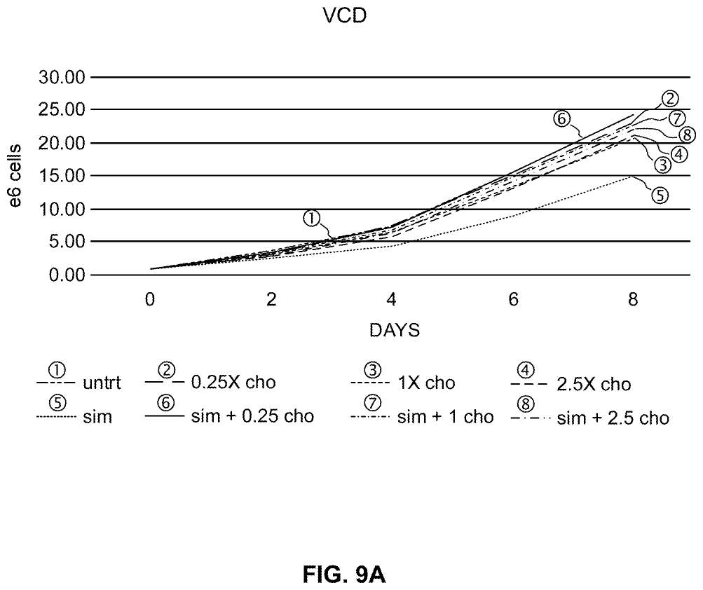

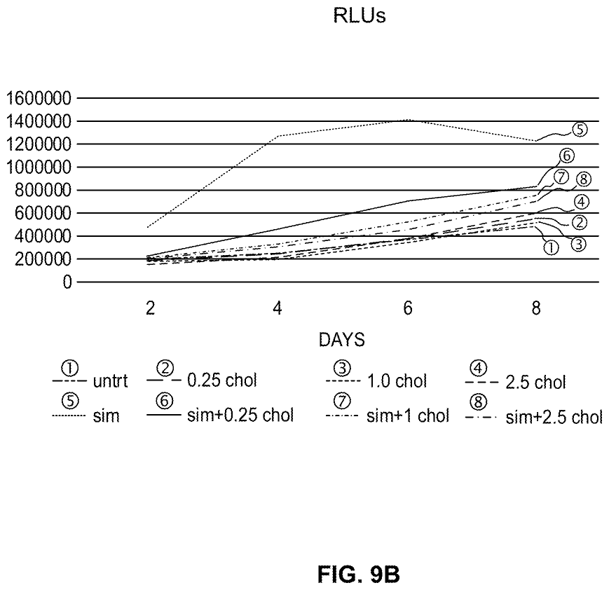

To confirm the importance of cholesterol depletion on enhanced exosome production, the endogenous cholesterol biosynthetic pathway in the HiBit reporter cell line was inhibited by the addition of simvastatin to the cell culture media while at the same time supplementing the media with varying concentrations of synthetic cholesterol. It was expected that replacement of endogenous cholesterol by synthetic cholesterol would reverse the effect of simvastatin. Depletion of cellular cholesterol by simvastatin caused a characteristic decrease in viable cell density (VCD; FIG. 9A) and an increase in exosome production (FIG. 9B) in HiBit cultures. Cell cultures grown with exogenous, synthetic cholesterol and 6 mM simvastatin displayed no decrease in VCD (FIG. 9A). Simvistatin induction of EV production was largely attenuated by the inclusion of supplemental cholesterol, though EV production was still observed to be higher than in control cultures lacking statin (FIG. 9B). This reversal was specific to the simvastatin effect, as growing cultures in elevated cholesterol alone had no impact on cell culture VCD or exosome production (FIGS. 9A and 9B).

### INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes.

### EQUIVALENTS

While various specific aspects have been illustrated and described, the above specification is not restrictive. It will be appreciated that various changes can be made without departing from the spirit and scope of the invention(s). Many variations will become apparent to those skilled in the art upon review of this specification.

What is claimed is:

1. A method of increasing a number of extracellular vesicles (EVs) produced from producer cells, comprising modifying the producer cells to exhibit a reduced gene and/or protein function in a cholesterol biosynthetic pathway of the producer cells.

2. A method of producing extracellular vesicles (EVs) from producer cells, comprising culturing the producer cells, which exhibit a reduced gene and/or protein function in a cholesterol biosynthetic pathway.

3. The method of claim 1 or 2, wherein the reduced gene and/or protein function in a cholesterol biosynthesis comprises one or more genes selected from 3-hydroxy-3-methylglutaryl-coenzyme A reductase (HMGCR), sterol regulatory element-binding protein 2 (SREBF2), Squalene epoxidase (SQLE), or 7-Dehydrocholesterol reductase (DHCR7) or a protein encoded by the gene.

4. The method of any one of claims 1 to 3, wherein the EVs produced by the producer cells have an increased yield compared to EVs produced by producer cells that the gene and/or protein function in a cholesterol biosynthetic pathway is not reduced.

5. The method of claim 4, wherein the yield is increased at least about 1.5 fold, at least about 2 fold, at least about 3 fold, at least about 4 fold, at least about 5 fold, at least about 6 fold, at least about 7 fold, at least about 8 fold, at least about 9 fold, at least about 10 fold, at least about 11 fold, at least about 12 fold, at least about 13 fold, at least about 14 fold, at least about 15 fold, at least about 16 fold, at least about 17 fold, at least about 18 fold, at least about 19 fold, at least about 20 fold. at least about 21 fold, at least about 22 fold, at least about 23 fold, at least about 24 fold, at least about 25 fold, at least about 26 fold, at least about 27 fold, at least about 28 fold, at least about 29 fold, or at least about 30 fold.

6. The method of claim 4, wherein the yield is increased about 2 fold to about 30 fold, about 2 fold to about 25 fold, about 2 fold to about 20 fold, about 2 fold to about 15 fold, about 2 fold to about 10 fold, about 2 fold to about 5 fold, about 5 fold to about 30 fold, about 5 fold to about 25 fold, about 5 fold to about 20 fold, about 5 fold to about 15 fold, about 5 fold to about 10 fold, about 10 fold to about 30 fold, about 10 fold to about 25 fold, about 10 fold to about 20 fold, about 10 fold to about 15 fold, about 15 fold to about 30 fold, about 15 fold to about 25 fold, about 15 fold to about 20 fold, about 20 fold to about 30 fold, about 20 fold to about 25 fold, or about 25 fold to about 30 fold.

7. The method of any one of claims 4-6, wherein the yield is increased about 2 fold, about 3 fold, about 4 fold, about 5 fold, about 6 fold, about 7 fold, about 8 fold, about 9 fold, or about 10 fold.

8. The method of any one of claims 1 to 7, wherein the reduced gene in a cholesterol biosynthesis is SREBF2.

9. The method of claim 8, wherein the SREBF2 gene expression is reduced about 2 fold to about 20 fold.

10. The method of claim 9, wherein the SREBF2 gene expression is reduced about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, or about 20 fold.

11. The method of any one of claims 1 to 7, wherein the reduced gene in a cholesterol biosynthesis is HMGCR.

12. The method of claim 11, wherein the HMGCR gene expression is reduced about 2 fold to 30 fold.

13. The method of claim 12, wherein the HMGCR gene expression is reduced about 2 fold, about 3 fold, about 4 fold, about 5 fold, about 6 fold, about 7 fold, about 8 fold, about 9 fold, about 10 fold, about 11 fold, about 12 fold, about 13 fold, about 14 fold, about 15 fold, about 16 fold, about 17 fold, about 18 fold, about 19 fold, about 20 fold, about 21 fold, about 22 fold, about 23 fold, about 24 fold, about 25 fold, about 26 fold, about 27 fold, about 28 fold, about 29 fold, or about 30 fold.

14. The method of any one of claims 1-13, wherein the gene and/protein function in a cholesterol biosynthetic pathway is reduced at least about 90%, at least about 80%, at least about 70%, at least about 60%, at least about 50%, at least about 40%, at least about 30%, at least about 20%, or at least about 10%, at least about 5%, or at least about 1%.

15. The method of any one of claims 1-14, wherein the gene and/protein function in a cholesterol biosynthetic pathway is reduced at least about 90%, at least about 80%, at least about 70%, at least about 60%, at least about 50%, at least about 40%, or at least about 30%.

16. The method of any one of claims 1 and 3-15, wherein the modifying comprises contacting the producer cells with an agent capable of reducing the gene and/or protein function in a cholesterol biosynthetic pathway.

17. The method of any one of claims 2-15, wherein the producer cells are modified prior to the culturing by contacting the producer cells with an agent capable of reducing the gene and/or protein function in a cholesterol biosynthetic pathway.

18. The method of claim 16 or 17, wherein the agent comprises a statin, a cariprazine, a PROTAC, AY9944, or BM15766.

19. The method of claim 18, wherein the statin comprises atorvastatin, lovastatin, pitavastatin, pravastatin, fluvastatin, cerivastatin, rosuvastatin, simvastatin, or combinations thereof.

20. The method of claims 18 or 19, wherein the statin is contacted at a concentration of about 1 nM, about 2 nM, about 3 nM, about 4 nM, about 5 nM, about 6 nM, about 7 nM, about 8 nM, about 9 nM, about 10 nM, about 11 nM, about 12 nM, about 13 nM, about 14 nM, about 15 nM, about 16 nM, about 17 nM, about 18 nM, or about 20 nM, about 30 nM, about 40 nM, about 50 nM, about 60 nM, about 70 nM, about 80 nM, about 90 nM, about 100 nM, about 110 nM, about 120 nM, about 130 nM, about 140 nM, about 150 nM, about 160 nM, about 170 nM, about 180 nM, about 190 nM, about 200 nM, about 210 nM, about 220 nM, about 230 nM, about 240 nM, about 250 nM, about 260 nM, about 270 nM, about 280 nM, about 290 nM, about 300 nM, about 310 nM, about 320 nM, about 330 nM, about 340 nM, about 350 nM, about 360 nM, about 370 nM, about 380 nM, about 390 nM, about 400 nM, about 410 nM, about 420 nM, about 430 nM, about 440 nM, about 450 nM, about 460 nM, about 470 nM, about 480 nM, about 490 nM, about 500 nM, about 510 nM, about 520 nM, about 530 nM, about 540 nM, about 550 nM, about 560 nM, about 570 nM, about 580 nM, about 590 nM, about 600 nM, about 610 nM, about 620 nM, about 630 nM, about 640 nM, about 650 nM, about 660 nM, about 670 nM, about 680 nM, about 690 nM, about 700 nM, about 710 nM, about 720 nM, about 730 nM, about 740 nM, about 750 nM, about 760 nM, about 770 nM, about 780 nM, about 790 nM, about 800 nM, about 810 nM, about 820 nM, about 830 nM, about 840 nM, about 850 nM, about 860 nM, about 870 nM, about 880 nM, about 890 nM, about 900 nM, about 910 nM, about 920 nM, about 930 nM, about 940 nM, about 950 nM, about 960 nM, about 970 nM, about 980 nM, about 990 nM, or about 1,000 nM.

21. The method of claim 18 or 19, wherein the statin is contacted at a concentration of between about 0.1 nM to about 100 nM, about 0.1 nM to about 90 nM, about 0.1 nM to about 80 nM, about 0.1 nM to about 70 nM, about 0.1 nM to about 60 nM, about 0.1 nM to about 50 nM, about 0.1 nM to about 40 nM, about 0.1 nM to about 30 nM, about 0.1 nM to about 20 nM, 0.1 nM to about 10 nM, or about 1 nM to about 20 nM, about 1 nM to about 10 nM, about 1 nM to about 5 nM, about 5 nM to about 20 nM, about 5 nM to about 15 nM, about 5 nM to about 10 nM, about 10 nM to about 50 nM, about 10 nM to about 40 nM, about 10 nM to about 30 nM, about 10 nM to about 20 nM, about 1 nM to about 10 nM, or about 10 nM to about 20 nM.

22. The method of claim 16 or 17, wherein the agent capable of reducing the gene and/or protein function in a cholesterol biosynthetic pathway comprises a gene editing technology.

23. The method of claim 22, wherein the gene editing technology comprises a shRNA, siRNA, miRNA, antisense oligonucleotides, CRISPR, zinc finger nuclease, TALEN, meganuclease, restriction endonuclease, or any combination thereof.

24. The method of claim 23, wherein the gene editing technology comprises siRNA.

25. The method of any one of claims 1-24, wherein the EVs produced by the producer cells have decreased cholesterol content per EV compared to EVs produced by producer cells that the gene and/or protein function in a cholesterol biosynthetic pathway is not reduced.

26. The method of claim 25, wherein the cholesterol content per EV is reduced by about 10%, about 20%, about 30%, about 40%, about 50%, about 60%, about 70%, or about 80%.

27. The method of claim 26, wherein the cholesterol content per EV is reduced by about 1% to about 80%, about 10% to about 80%, about 10% to about 70%, about 10% to about 60%, about 10% to about 50%, about 10% to about 40%, about 10% to about 30%, about 10% to about 20%, about 20% to about 80%, about 20% to about 70%, about 20% to about 60%, about 20% to about 50%, about 20% to about 40%, about 20% to about 30%, about 30% to about 80%, about 30% to about 70%, about 30% to about 60%, about 30% to about 50%, about 30% to about 40%, about 40% to about 80%, about 40% to about 70%, about 40% to about 60%, or about 40% to about 50%.

28. The method of any one of claims 1-27, wherein the EVs produced by the producer cells do not have a difference in average size distribution compared to EVs produced by producer cells that the gene and/or protein function in a cholesterol biosynthetic pathway is not reduced.

29. The method of any one of claims 1-28, wherein the producer cells are mammalian cells.

30. The method of any one of claims 1-29, wherein the producer cells are HEK293 cells, HEK293S cells, HEK293SF cells, Chinese Hamster Ovary (CHO) cells, mesenchymal stem cells (MSCs), BJ human foreskin fibroblast cells, fHIDF fibroblast cells, AGE.HN® neuronal precursor cells, CAP® amniocyte cells, adipose mesenchymal stem cells, RPTEC/TERT1 cells, dendritic cells, macrophages, B cells, mast cells, neutrophils, Kupffer-Browicz cells, PER.C6 cells, Induced pluripotent stem cells (iPSCs), or C2C12 cells.

31. The method of any one of claims 1-29, wherein the producer cells are stem cells.

32. The method of any one of claims 1-31, wherein the EVs further comprise a scaffold moiety.

33. The method of claim 32, wherein the scaffold moiety comprises a Scaffold X.

34. The method of claim 33, wherein Scaffold X is selected from the group consisting of prostaglandin F2 receptor negative regulator (the PTGFRN protein); basigin (the BSG protein); immunoglobulin superfamily member 2 (the IGSF2 protein); immunoglobulin superfamily member 3 (the IGSF3 protein); immunoglobulin superfamily member 8 (the IGSF8 protein); integrin beta-1 (the ITGB1 protein); integrin alpha-4 (the ITGA4 protein); 4F2 cell-surface antigen heavy chain (the SLC3A2 protein); a class of ATP transporter proteins (the ATP1A1, ATP1A2, ATP1A3, ATP1A4, ATP1B3, ATP2B1, ATP2B2, ATP2B3, ATP2B4 proteins), and any combination thereof.

35. The method of claim 34, wherein the scaffold moiety is a PTGFRN protein.

36. The method of claim 35, wherein the scaffold moiety comprises an amino acid sequence having at least 50%, at least 60%, at least 70%, at least 80%, at least 85%, at least 90%, at least 95%, at least 96%, at least 97%, at least 98%, at least 99%, or about 100% sequence identity to SEQ ID NO: 1.

37. The method of claim 32, wherein the scaffold moiety comprises a Scaffold Y.

38. The method of claim 37, wherein the Scaffold Y is selected from the group consisting of myristoylated alanine rich Protein Kinase C substrate (the MARCKS protein); myristoylated alanine rich Protein Kinase C substrate like 1 (the MARCKSL1 protein); brain acid soluble protein 1 (the BASP1 protein), and any combination thereof.

39. The method of any one of claims 32 to 38, wherein the EV further comprises at least a therapeutic agent linked to a scaffold moiety.

40. The method of any one of claims 1-39, wherein the EV further comprises at least a therapeutic agent.

41. The method of claim 39 or 40, wherein the therapeutic agent comprises a cytokine, a small molecule, a growth factor, an antigen, an antisense oligonucleotide, an siRNA, an shRNA, a miRNA, a dsDNA, a lncRNA, a PROTAC, an adjuvant, an immune modulator, or any combination thereof.

42. The method of claim 40, wherein the therapeutic agent is IL-12.

43. The method of claim 40, wherein the therapeutic agent is a STING agonist.

44. The method of claim 40, wherein the therapeutic agent is an antisense oligonucleotide.

45. The method of claim 44, wherein the antisense oligonucleotide targets Kras, STAT3, Nras, STAT6, CEBP b, NLRP3, or any combination thereof.

46. Producer cells for use in the method of any one of claims 1 to 45.

47. Producer cells prepared by the method of any one of claims 1 and 3 to 45.

48. Extracellular vesicles produced by the method of any one of claims 1 to 45 or the producer cells of claim 46 or 47.

49. A bioreactor comprising the producer cells of claim 46 or 47, or extracellular vesicles of claim 48.

50. A method of treating or preventing a disease or a condition in a subject in need thereof comprising administering the extracellular vesicles of claim 48.

51. Use of the extracellular vesicles of claim 48 to treat or prevent a disease or condition in a subject in need thereof.

52. Extracellular vesicles of claim 48 for treating or preventing a disease or condition in a subject in need thereof.
